(19) United States
(12) Patent Application Publication
(10) Pub. No.: US 2023/0233555 A1
(43) Pub. Date: Jul. 27, 2023

(54) METHOD OF ADMINISTERING UPADACITINIB TO AVOID ADVERSE DRUG INTERACTIONS AND EFFECTS

(71) Applicant: AbbVie Inc., North Chicago, IL (US)

(72) Inventor: Mohamed-Eslam F. Mohamed, Gurnee, IL (US)

(73) Assignee: AbbVie Inc., North Chicago, IL (US)

(21) Appl. No.: 18/127,211

(22) Filed: Mar. 28, 2023

Related U.S. Application Data
(63) Continuation of application No. PCT/US2023/060648, filed on Jan. 13, 2023.
(60) Provisional application No. 63/320,149, filed on Mar. 15, 2022.
(60) Provisional application No. 63/299,324, filed on Jan. 13, 2022.

(51) Int. Cl. A61K 31/4985; A61K 31/496; A61P 1/00

(52) U.S. Cl. CPC A61K 31/4985; A61K 31/496; A61P 1/00

(57) ABSTRACT

The present disclosure is directed to methods for treating immunological diseases and disorders with upadacitinib. The methods include adjusting the dose of upadacitinib in patients with severe renal impairment, in patients concurrently receiving a strong inhibitor of cytochrome P450 3A4 (CYP3A4), and in adult patients 65 years of age and older.

### CROSS-REFERENCE TO RELATED APPLICATIONS

This application is a continuation of International Application No. PCT/US23/60648, filed Jan. 13, 2023, and which claims the benefit of U.S. Provisional Application No. 63/299,324, filed Jan. 13, 2022, and claims the benefit of U.S. Provisional Application No. 63/320,149, filed Mar. 15, 2022, each of which are herein incorporated by reference in their entirety.

### FIELD OF THE DISCLOSURE

The present disclosure is directed to methods for treating diseases and disorders with upadacitinib.

### BACKGROUND OF THE DISCLOSURE

Upadacitinib is a novel JAK1 selective inhibitor with minimal inhibitory effects on JAK2 and JAK3, which could potentially minimize some of the reported safety concerns with non-selective JAK inhibition which are thought to be mediated by inhibition of JAK2 and JAK3 signaling pathways, such as infections, including herpes zoster reactivation, malignancies, and asymptomatic, mild and reversible changes in levels of hemoglobin, lymphocyte counts, white blood cell counts, serum creatinine, total cholesterol, high density lipoprotein cholesterol (HDL-C), low density lipoprotein cholesterol (LDL-C) and liver transaminases (alanine transaminase[ALT], aspartate transaminase [AST]) and creatine phosphokinase (CPK). See Vermeire et al. “Clinical remission in patients with moderate-to-severe Crohn's disease treated with filgotinib (the FITZROY study): results from a phase 2, double-blind, randomized, placebo-controlled trial.” Lancet 2017; 389(10066):266-75; Fleischmann et al; “ORAL Solo Investigators. Placebo-controlled trial of tofacitinib monotherapy in rheumatoid arthritis.” N Engl J Med. 2012; 367(6):495-507).

JAK1 inhibition blocks the signaling of many important pro-inflammatory cytokines, including interleukin (IL)-2, IL-6, IL-7, and IL-15, which are known contributors to inflammatory disorders. Through modulation of these pro-inflammatory cytokine pathways, upadacitinib offers the potential for effective treatment of inflammatory or autoimmune disorders such as rheumatoid arthritis (RA), psoriatic arthritis (PsA), axial spondyloarthritis (axSpA), giant cell arteritis (GCA), Takayasu arteritis, polyarticular course juvenile idiopathic arthritis (pcJIA), Crohn's disease (CD), UC and atopic dermatitis (AD). The clinical hypothesis is that, based on the differentiated selectivity profile for JAK inhibition, upadacitinib could demonstrate an improved benefit/risk profile compared to other less selective JAK inhibitors or other therapeutic strategies for patients with inflammatory diseases.

To date, upadacitinib has been investigated in 22 Phase 1 studies with healthy volunteers (one of which also employed a sub-study in subjects with mild to moderate RA), and multiple Phase 2 or Phase 3 studies for indications of RA, CD, AD, PsA and UC. Upadacitinib has been shown to be effective in the treatment of the indicated medical conditions based on the data available from the various studies. Safety results across the studies showed that upadacitinib was well tolerated and the types and frequencies of AEs were consistent with subjects with the medical conditions receiving immunomodulatory therapy.

### SUMMARY OF THE DISCLOSURE

As described herein, upadacitinib is well tolerated and has proven efficacious in multiple diseases and disorders across numerous clinical studies. During the clinical development of upadacitinib, it was unexpectedly observed that concurrent administration with certain therapeutic agents, or in patients with renal insufficiency, may result in increased or decreased systemic exposure of upadacitinib. In such instances, it may be desirable to adjust the upadacitinib dose or avoid administration of therapeutic agents which alter the exposure level of upadacitinib. The present disclosure addresses these needs, providing methods for treating diseases and disorders with upadacitinib while avoiding potentially adverse interactions.

In one aspect, the present disclosure provides a method of treating patients concurrently receiving a strong CYP3A4 inhibitor with upadacitinib, the method comprising:
reducing a recommended daily upadacitinib dose of 45 mg to 30 mg during the time the patients are receiving the strong CYP3A4 inhibitor;
reducing a recommended daily upadacitinib dose of 30 mg to 15 mg during the time the patients are receiving the strong CYP3A4 inhibitor; and
maintaining a recommended daily upadacitinib dose of 15 mg during the time the patients are receiving the strong CYP3A4 inhibitor.

In another aspect, the present disclosure provides a method for treating a disease clinically approved for treatment with upadacitinib in patients receiving a strong CYP3A4 inhibitor, the method comprising:
reducing a recommended daily upadacitinib dose of 45 mg to 30 mg during the time the patients are receiving the strong CYP3A4 inhibitor;
reducing a recommended daily upadacitinib dose of 30 mg to 15 mg during the time the patients are receiving the strong CYP3A4 inhibitor; and
maintaining a recommended daily upadacitinib dose of 15 mg during the time the patients are receiving the strong CYP3A4 inhibitor.

In yet another aspect, the present disclosure provides a method of improving safety when administering upadacitinib to patients receiving a strong CYP3A4 inhibitor, the method comprising:
reducing a recommended daily upadacitinib dose of 45 mg to 30 mg during the time the patients are receiving the strong CYP3A4 inhibitor;
reducing a recommended daily upadacitinib dose of 30 mg to 15 mg during the time the patients are receiving the strong CYP3A4 inhibitor; and
maintaining a recommended daily upadacitinib dose of 15 mg during the time the patients are receiving the strong CYP3A4 inhibitor.

In a further aspect, the present disclosure provides a method of reducing an occurrence of or potential for adverse events in treating a disease clinically approved for treatment with upadacitinib in patients receiving a strong CYP3A4 inhibitor, the method comprising:
reducing a recommended daily upadacitinib dose of 45 mg to 30 mg during the time the patients are receiving the strong CYP3A4 inhibitor;
reducing a recommended daily upadacitinib dose of 30 mg to 15 mg during the time the patients are receiving the strong CYP3A4 inhibitor; and
maintaining a recommended daily upadacitinib dose of 15 mg during the time the patients are receiving the strong CYP3A4 inhibitor.

In a yet further aspect, the present disclosure provides a method of treating an FDA approved indication with upadacitinib in patients receiving a strong CYP3A4 inhibitor, the method comprising:
reducing a recommended daily upadacitinib dose of 45 mg to 30 mg during the time the patients are receiving the strong CYP3A4 inhibitor;
reducing a recommended daily upadacitinib dose of 30 mg to 15 mg during the time the patients are receiving the strong CYP3A4 inhibitor; and
maintaining a recommended daily upadacitinib dose of 15 mg during the time the patients are receiving the strong CYP3A4 inhibitor.

In some embodiments, the daily upadacitinib dose for patients not receiving a strong CYP3A4 inhibitor is 45 mg, which is reduced to a daily upadacitinib dose of 30 mg during the time the patient is receiving the strong CYP3A4 inhibitor. In some embodiments, the daily upadacitinib dose of 30 mg during the time the patient is receiving the strong CYP3A4 inhibitor is administered without need for caution. In some embodiments, the patient is closely monitored for adverse reactions when administered the daily upadacitinib dose of 30 mg during the time the patient is receiving the strong CYP3A4 inhibitor. In some embodiments, the daily upadacitinib dose of 30 mg is administered as two 15 mg dosage forms.

In some embodiments, the daily upadacitinib dose for patients not receiving a strong CYP3A4 inhibitor is 30 mg, which is reduced to a daily upadacitinib dose of 15 mg during the time the patient is receiving the strong CYP3A4 inhibitor. In some embodiments, the daily upadacitinib dose of 15 mg during the time the patient is receiving the strong CYP3A4 inhibitor is administered without need for caution. In some embodiments, the patient is closely monitored for adverse reactions when administered the daily upadacitinib dose of 15 mg during the time the patient is receiving the strong CYP3A4 inhibitor.

In some embodiments, the daily upadacitinib dose for patients not receiving a strong CYP3A4 inhibitor is 15 mg, which is maintained during the time the patient is receiving the strong CYP3A4 inhibitor. In some embodiments, the daily upadacitinib dose of 15 mg during the time the patient is receiving the strong CYP3A4 inhibitor is administered without need for caution. In some embodiments, the patient is closely monitored for adverse reactions when administered the daily upadacitinib dose of 15 mg during the time the patient is receiving the strong CYP3A4 inhibitor.

In some embodiments, the daily upadacitinib dose for patients not receiving a strong CYP3A4 inhibitor is 15 mg, which is maintained during the time the patient is receiving the strong CYP3A4 inhibitor. In some embodiments, the daily upadacitinib dose of 15 mg during the time the patient is receiving the strong CYP3A4 inhibitor is administered without need for caution. In some embodiments, the patient is closely monitored for adverse reactions when administered the daily upadacitinib dose of 15 mg during the time the patient is receiving the strong CYP3A4 inhibitor.

In some embodiments, the disease clinically approved for treatment with upadacitinib is rheumatoid arthritis. In some embodiments, the disease clinically approved for treatment with upadacitinib is rheumatoid arthritis, and the daily upadacitinib dose of 15 mg is maintained. In some embodiments, the patient is a rheumatoid arthritis patient and the daily upadacitinib dose of 15 mg during the time the patient is receiving the strong CYP3A4 inhibitor is administered without need for caution. In some embodiments, the disease clinically approved for treatment with upadacitinib is psoriatic arthritis. In some embodiments, the disease clinically approved for treatment with upadacitinib is psoriatic arthritis, and the daily upadacitinib dose of 15 mg is maintained.

In some embodiments, the disease clinically approved for treatment with upadacitinib is atopic dermatitis. In some embodiments, the disease clinically approved for treatment with upadacitinib is atopic dermatitis, and the daily upadacitinib dose of 15 mg is maintained. In some embodiments, the disease clinically approved for treatment with upadacitinib is atopic dermatitis, and the daily upadacitinib dose of 30 mg is reduced to 15 mg.

In some embodiments, the disease clinically approved for treatment with upadacitinib is ulcerative colitis. In some embodiments, the disease clinically approved for treatment with upadacitinib is ulcerative colitis, and the daily upadacitinib dose of 45 mg is reduced to 30 mg. In some embodiments, the disease clinically approved for treatment with upadacitinib is ulcerative colitis, and the daily upadacitinib dose of 30 mg may be considered for patients with refractory, severe, or extensive disease.

In some embodiments, the disease clinically approved for treatment with upadacitinib is Crohn's disease. In some embodiments, the disease clinically approved for treatment with upadacitinib is Crohn's disease, and the daily upadacitinib dose of 45 mg is reduced to 30 mg. In some embodiments, the disease clinically approved for treatment with upadacitinib is Crohn's disease, and the daily upadacitinib dose of 30 mg may be considered for patients with refractory, severe, or extensive disease.

In one aspect, the present disclosure provides a method for treating a disease clinically approved for treatment with upadacitinib in patients receiving a strong CYP34A inhibitor, the method comprising:
determining the recommended the daily upadacitinib dose for patients not receiving a strong CYP34A inhibitor, selected from 45 mg, 30 mg or 15 mg;
reducing the recommended daily upadacitinib dose from 45 mg to 30 mg during the time the patients are receiving the strong CYP3A4 inhibitor;
reducing the recommended daily upadacitinib dose from 30 mg to 15 mg during the time the patients are receiving the strong CYP3A4 inhibitor; and
maintaining the recommended daily upadacitinib dose of 15 mg during the time the patients are receiving the strong CYP3A4 inhibitor.

In another aspect, the present disclosure provides a method for treating a disease clinically approved for treatment with upadacitinib in patients receiving a strong CYP34A inhibitor, the method comprising:
assessing whether the patients are receiving a strong CYP34A inhibitor;
reducing a recommended daily upadacitinib dose of 45 mg to 30 mg during the time the patients are receiving the strong CYP3A4 inhibitor;
reducing a recommended daily upadacitinib dose of 30 mg to 15 mg during the time the patients are receiving the strong CYP3A4 inhibitor; and
maintaining a recommended daily upadacitinib dose of 15 mg during the time the patients are receiving the strong CYP3A4 inhibitor.

In one aspect, the present disclosure provides a method for treating a disease clinically approved for treatment with upadacitinib in patients receiving a strong CYP3A4 inhibitor, the method comprising:
determining the recommended the daily upadacitinib dose for patients not receiving a strong CYP3A4 inhibitor, selected from 45 mg, 30 mg or 15 mg;
assessing whether the patients are receiving a strong CYP3A4 inhibitor;
reducing the recommended daily upadacitinib dose from 45 mg to 30 mg during the time the patients are receiving the strong CYP3A4 inhibitor;
reducing the recommended daily upadacitinib dose from 30 mg to 15 mg during the time the patients are receiving the strong CYP3A4 inhibitor; and
maintaining the recommended daily upadacitinib dose of 15 mg during the time the patients are receiving the strong CYP3A4 inhibitor.

In some embodiments, the daily upadacitinib dose for patients not receiving a strong CYP3A4 inhibitor is 45 mg, which is reduced to a daily upadacitinib dose of 30 mg during the time the patient is receiving the strong CYP3A4 inhibitor. In some embodiments, the daily upadacitinib dose of 30 mg during the time the patient is receiving the strong CYP3A4 inhibitor is administered without need for caution. In some embodiments, the patient is closely monitored for adverse reactions when administered the daily upadacitinib dose of 30 mg during the time the patient is receiving the strong CYP3A4 inhibitor. In some embodiments, the daily upadacitinib dose of 30 mg is administered as two 15 mg dosage forms.

In some embodiments, the daily upadacitinib dose for patients not receiving a strong CYP3A4 inhibitor is 30 mg, which is reduced to a daily upadacitinib dose of 15 mg during the time the patient is receiving the strong CYP3A4 inhibitor. In some embodiments, the daily upadacitinib dose of 15 mg during the time the patient is receiving the strong CYP3A4 inhibitor is administered without need for caution. In some embodiments, the patient is closely monitored for adverse reactions when administered the daily upadacitinib dose of 15 mg during the time the patient is receiving the strong CYP3A4 inhibitor.

In some embodiments, the daily upadacitinib dose for patients not receiving a strong CYP3A4 inhibitor is 15 mg, which is maintained during the time the patient is receiving the strong CYP3A4 inhibitor. In some embodiments, the daily upadacitinib dose of 15 mg during the time the patient is receiving the strong CYP3A4 inhibitor is administered without need for caution. In some embodiments, the patient is closely monitored for adverse reactions when administered the daily upadacitinib dose of 15 mg during the time the patient is receiving the strong CYP3A4 inhibitor.

In some embodiments, the daily upadacitinib dose for patients not receiving a strong CYP3A4 inhibitor is 15 mg, which is maintained during the time the patient is receiving the strong CYP3A4 inhibitor. In some embodiments, the daily upadacitinib dose of 15 mg during the time the patient is receiving the strong CYP3A4 inhibitor is administered without need for caution. In some embodiments, the patient is closely monitored for adverse reactions when administered the daily upadacitinib dose of 15 mg during the time the patient is receiving the strong CYP3A4 inhibitor.

In some embodiments, the disease clinically approved for treatment with upadacitinib is rheumatoid arthritis. In some embodiments, the disease clinically approved for treatment with upadacitinib is rheumatoid arthritis, and the daily upadacitinib dose of 15 mg is maintained. In some embodiments, the patient is a rheumatoid arthritis patient and the daily upadacitinib dose of 15 mg during the time the patient is receiving the strong CYP3A4 inhibitor is administered without need for caution. In some embodiments, the disease clinically approved for treatment with upadacitinib is psoriatic arthritis. In some embodiments, the disease clinically approved for treatment with upadacitinib is psoriatic arthritis, and the daily upadacitinib dose of 15 mg is maintained.

In some embodiments, the disease clinically approved for treatment with upadacitinib is atopic dermatitis. In some embodiments, the disease clinically approved for treatment with upadacitinib is atopic dermatitis, and the daily upadacitinib dose of 15 mg is maintained. In some embodiments, the disease clinically approved for treatment with upadacitinib is atopic dermatitis, and the daily upadacitinib dose of 30 mg is reduced to 15 mg.

In some embodiments, the disease clinically approved for treatment with upadacitinib is ulcerative colitis. In some embodiments, the disease clinically approved for treatment with upadacitinib is ulcerative colitis, and the daily upadacitinib dose of 45 mg is reduced to 30 mg. In some embodiments, the disease clinically approved for treatment with upadacitinib is ulcerative colitis, and the daily upadacitinib dose of 30 mg is reduced to 15 mg.

In some embodiments, the disease clinically approved for treatment with upadacitinib is Crohn's disease. In some embodiments, the disease clinically approved for treatment with upadacitinib is Crohn's disease, and the daily upadacitinib dose of 45 mg is reduced to 30 mg. In some embodiments, the disease clinically approved for treatment with upadacitinib is Crohn's disease, and the daily upadacitinib dose of 30 mg may be considered for patients with refractory, severe, or extensive disease.

In another aspect, the present disclosure provides a method for treating a disease clinically approved for treatment with upadacitinib in patients receiving a strong CYP3A4 inhibitor, the method comprising:
determining the recommended the daily upadacitinib dose for patients not receiving a strong CYP3A4 inhibitor, selected from 45 mg, 30 mg or 15 mg;
assessing whether the patients are receiving a strong CYP3A4 inhibitor;
administering a reduced daily upadacitinib dose of 30 mg to patients receiving a strong CYP3A4 inhibitor and a daily upadacitinib dose of 45 mg during the time the patients are receiving the strong CYP3A4 inhibitor;
administering a reduced daily upadacitinib dose of 15 mg to patients receiving a strong CYP3A4 inhibitor and a daily upadacitinib dose of 30 mg during the time the patients are receiving the strong CYP3A4 inhibitor; and
administering a daily upadacitinib dose of 15 mg to patients receiving a strong CYP3A4 inhibitor and a daily upadacitinib dose of 15 mg during the time the patients are receiving the strong CYP3A4 inhibitor.

In one aspect, the present disclosure provides a method of treating a patient concurrently receiving a strong CYP3A4 inhibitor with upadacitinib, the method comprising:
reducing a recommended daily upadacitinib dose of 45 mg to 30 mg during the time the patient is receiving the strong CYP3A4 inhibitor;
reducing a recommended daily upadacitinib dose of 30 mg to 15 mg during the time the patient is receiving the strong CYP3A4 inhibitor; or
maintaining a recommended daily upadacitinib dose of 15 mg during the time the patient is receiving the strong CYP3A4 inhibitor.

In another aspect, the present disclosure provides a method for treating a disease clinically approved for treatment with upadacitinib in a patient receiving a strong CYP3A4 inhibitor, the method comprising:
determining the recommended the daily upadacitinib dose when the patient is not receiving a strong CYP3A4 inhibitor, selected from 45 mg, 30 mg or 15 mg;
assessing whether the patient is receiving a strong CYP3A4 inhibitor; and
(a) reducing the recommended daily upadacitinib dose of 45 mg to a daily upadacitinib dose of 30 mg during the time the patient is receiving the strong CYP3A4 inhibitor,
(b) reducing the recommended daily upadacitinib dose of 30 mg to a daily upadacitinib dose of 15 mg during the time the patient is receiving the strong CYP3A4 inhibitor, or
(c) maintaining the recommended daily upadacitinib dose of 15 mg during the time the patient is receiving the strong CYP3A4 inhibitor.

In another aspect, the present disclosure provides a method for treating a disease clinically approved for treatment with upadacitinib in a patient receiving a strong CYP3A4 inhibitor, the method comprising:
determining the recommended the daily upadacitinib dose when the patient is not receiving a strong CYP3A4 inhibitor, selected from 45 mg, 30 mg or 15 mg;
assessing whether the patient is receiving a strong CYP3A4 inhibitor; and
(a) if the patient is receiving a strong CYP3A4 inhibitor and a daily upadacitinib dose of 45 mg, administering a daily upadacitinib dose of 30 mg during the time the patient is receiving the strong CYP3A4 inhibitor;
(b) if the patient is receiving a strong CYP3A4 inhibitor and a daily upadacitinib dose of 30 mg, administering a daily upadacitinib dose of 15 mg during the time the patient is receiving the strong CYP3A4 inhibitor; or
(c) if the patient is receiving a strong CYP3A4 inhibitor and a daily upadacitinib dose of 15 mg, administering a daily upadacitinib dose of 15 mg during the time the patient is receiving the strong CYP3A4 inhibitor.

In some embodiments, the daily upadacitinib dose for patients not receiving a strong CYP3A4 inhibitor is 45 mg, which is reduced to a daily upadacitinib dose of 30 mg during the time the patient is receiving the strong CYP3A4 inhibitor. In some embodiments, the daily upadacitinib dose of 30 mg during the time the patient is receiving the strong CYP3A4 inhibitor is administered without need for caution. In some embodiments, the patient is closely monitored for adverse reactions when administered the daily upadacitinib dose of 30 mg during the time the patient is receiving the strong CYP3A4 inhibitor. In some embodiments, the daily upadacitinib dose of 30 mg is administered as two 15 mg dosage forms.

In some embodiments, the daily upadacitinib dose for patients not receiving a strong CYP3A4 inhibitor is 30 mg, which is reduced to a daily upadacitinib dose of 15 mg during the time the patient is receiving the strong CYP3A4 inhibitor. In some embodiments, the daily upadacitinib dose of 15 mg during the time the patient is receiving the strong CYP3A4 inhibitor is administered without need for caution. In some embodiments, the patient is closely monitored for adverse reactions when administered the daily upadacitinib dose of 15 mg during the time the patient is receiving the strong CYP3A4 inhibitor.

In some embodiments, the daily upadacitinib dose for patients not receiving a strong CYP3A4 inhibitor is 15 mg, which is maintained during the time the patient is receiving the strong CYP3A4 inhibitor. In some embodiments, the daily upadacitinib dose of 15 mg during the time the patient is receiving the strong CYP3A4 inhibitor is administered without need for caution. In some embodiments, the patient is closely monitored for adverse reactions when administered the daily upadacitinib dose of 15 mg during the time the patient is receiving the strong CYP3A4 inhibitor.

In some embodiments, the daily upadacitinib dose for patients not receiving a strong CYP3A4 inhibitor is 15 mg, which is maintained during the time the patient is receiving the strong CYP3A4 inhibitor. In some embodiments, the daily upadacitinib dose of 15 mg during the time the patient is receiving the strong CYP3A4 inhibitor is administered without need for caution. In some embodiments, the patient is closely monitored for adverse reactions when administered the daily upadacitinib dose of 15 mg during the time the patient is receiving the strong CYP3A4 inhibitor.

In some embodiments, the disease clinically approved for treatment with upadacitinib is rheumatoid arthritis. In some embodiments, the disease clinically approved for treatment with upadacitinib is rheumatoid arthritis, and the daily upadacitinib dose of 15 mg is maintained. In some embodiments, the patient is a rheumatoid arthritis patient and the daily upadacitinib dose of 15 mg during the time the patient is receiving the strong CYP3A4 inhibitor is administered without need for caution. In some embodiments, the disease clinically approved for treatment with upadacitinib is psoriatic arthritis. In some embodiments, the disease clinically approved for treatment with upadacitinib is psoriatic arthritis, and the daily upadacitinib dose of 15 mg is maintained.

In some embodiments, the disease clinically approved for treatment with upadacitinib is atopic dermatitis. In some embodiments, the disease clinically approved for treatment with upadacitinib is atopic dermatitis, and the daily upadacitinib dose of 15 mg is maintained. In some embodiments, the disease clinically approved for treatment with upadacitinib is atopic dermatitis, and the daily upadacitinib dose of 30 mg is reduced to 15 mg.

In some embodiments, the disease clinically approved for treatment with upadacitinib is ulcerative colitis. In some embodiments, the disease clinically approved for treatment with upadacitinib is ulcerative colitis, and the daily upadacitinib dose of 45 mg is reduced to 30 mg. In some embodiments, the disease clinically approved for treatment with upadacitinib is ulcerative colitis, and the daily upadacitinib dose of 30 mg may be considered for patients with refractory, severe, or extensive disease.

In some embodiments, the disease clinically approved for treatment with upadacitinib is Crohn's disease. In some embodiments, the disease clinically approved for treatment with upadacitinib is Crohn's disease, and the daily upadacitinib dose of 45 mg is reduced to 30 mg. In some embodiments, the disease clinically approved for treatment with upadacitinib is Crohn's disease, and the daily upadacitinib dose of 30 mg may be considered for patients with refractory, severe, or extensive disease.

In a still further aspect, the present disclosure provides a method of treating patients having severe renal impairment with upadacitinib, the method comprising:
reducing a recommended daily upadacitinib dose of 45 mg to 30 mg during the time the patients have severe renal impairment;
reducing a recommended daily upadacitinib dose of 30 mg to 15 mg during the time the patients have severe renal impairment; and
maintaining a recommended daily upadacitinib dose of 15 mg during the time the patients have severe renal impairment.

In one aspect, the present disclosure provides a method for treating a disease clinically approved for treatment with upadacitinib in patients having severe renal impairment, the method comprising:
reducing a recommended daily upadacitinib dose of 45 mg to 30 mg during the time the patients have severe renal impairment;
reducing a recommended daily upadacitinib dose of 30 mg to 15 mg during the time the patients have severe renal impairment; and
maintaining a recommended daily upadacitinib dose of 15 mg during the time the patients have severe renal impairment.

In one aspect, the present disclosure provides a method for treating a disease clinically approved for treatment with upadacitinib in patients having severe renal impairment, the method comprising:
reducing a recommended daily upadacitinib dose of 45 mg to 30 mg during the time the patients have severe renal impairment;
reducing a recommended daily upadacitinib dose of 30 mg to 15 mg during the time the patients have severe renal impairment; and
maintaining a recommended daily upadacitinib dose of 15 mg during the time the patients have severe renal impairment.

In another aspect, the present disclosure provides a method for improving safety in treating a disease clinically approved for treatment with upadacitinib in patients having severe renal impairment, the method comprising:
reducing a recommended daily upadacitinib dose of 45 mg to 30 mg during the time the patients have severe renal impairment;
reducing a recommended daily upadacitinib dose of 30 mg to 15 mg during the time the patients have severe renal impairment; and
maintaining a recommended daily upadacitinib dose of 15 mg during the time the patients have severe renal impairment.

In yet another aspect, the present disclosure provides a method of reducing an occurrence of or potential for adverse events in treating a disease clinically approved for treatment with upadacitinib in patients having severe renal impairment, the method comprising:
reducing a recommended daily upadacitinib dose of 45 mg to 30 mg during the time the patients have severe renal impairment;
reducing a recommended daily upadacitinib dose of 30 mg to 15 mg during the time the patients have severe renal impairment; and
maintaining a recommended daily upadacitinib dose of 15 mg during the time the patients have severe renal impairment.

In a further aspect, the present disclosure provides a method of treating an FDA approved indication with upadacitinib in patients having severe renal impairment, the method comprising:
reducing a recommended daily upadacitinib dose of 45 mg to 30 mg during the time the patients have severe renal impairment;
reducing a recommended daily upadacitinib dose of 30 mg to 15 mg during the time the patients have severe renal impairment; and
maintaining a recommended daily upadacitinib dose of 15 mg during the time the patients have severe renal impairment.

In some embodiments, the daily upadacitinib dose for patients not having severe renal impairment is 45 mg, which is reduced to a daily upadacitinib dose of 30 mg during the time the patient is having severe renal impairment. In some embodiments, the daily upadacitinib dose of 30 mg during the time the patient is having severe renal impairment is administered without need for caution. In some embodiments, the patient is closely monitored for adverse reactions when administered the daily upadacitinib dose of 30 mg during the time the patient is having severe renal impairment. In some embodiments, the daily upadacitinib dose of 30 mg is administered as two 15 mg dosage forms.

In some embodiments, the daily upadacitinib dose for patients not having severe renal impairment is 30 mg, which is reduced to a daily upadacitinib dose of 15 mg during the time the patient is having severe renal impairment. In some embodiments, the daily upadacitinib dose of 15 mg during the time the patient is having severe renal impairment is administered without need for caution. In some embodiments, the patient is closely monitored for adverse reactions when administered the daily upadacitinib dose of 15 mg during the time the patient is having severe renal impairment.

In some embodiments, the daily upadacitinib dose for patients not having severe renal impairment is 15 mg, which is maintained during the time the patient is having severe renal impairment. In some embodiments, the daily upadacitinib dose of 15 mg during the time the patient is having severe renal impairment is administered without need for caution. In some embodiments, the patient is closely monitored for adverse reactions when administered the daily upadacitinib dose of 15 mg during the time the patient is having severe renal impairment.

In some embodiments, the daily upadacitinib dose for patients not having severe renal impairment is 15 mg, which is maintained during the time the patient is having severe renal impairment. In some embodiments, the daily upadacitinib dose of 15 mg during the time the patient is having severe renal impairment is administered without need for caution. In some embodiments, the patient is closely monitored for adverse reactions when administered the daily upadacitinib dose of 15 mg during the time the patient is having severe renal impairment.

In some embodiments, the disease clinically approved for treatment with upadacitinib is rheumatoid arthritis. In some embodiments, the disease clinically approved for treatment with upadacitinib is rheumatoid arthritis, and the daily upadacitinib dose of 15 mg is maintained. In some embodiments, the patient is a rheumatoid arthritis patient and the daily upadacitinib dose of 15 mg during the time the patient has severe renal impairment is administered without need for caution. In some embodiments, the disease clinically approved for treatment with upadacitinib is psoriatic arthritis. In some embodiments, the disease clinically approved for treatment with upadacitinib is psoriatic arthritis, and the daily upadacitinib dose of 15 mg is maintained.

In some embodiments, the disease clinically approved for treatment with upadacitinib is atopic dermatitis. In some embodiments, the disease clinically approved for treatment with upadacitinib is atopic dermatitis, and the daily upadacitinib dose of 15 mg is maintained. In some embodiments, the disease clinically approved for treatment with upadacitinib is atopic dermatitis, and the daily upadacitinib dose of 30 mg is reduced to 15 mg.

In some embodiments, the disease clinically approved for treatment with upadacitinib is ulcerative colitis. In some embodiments, the disease clinically approved for treatment with upadacitinib is ulcerative colitis, and the daily upadacitinib dose of 45 mg is reduced to 30 mg. In some embodiments, the disease clinically approved for treatment with upadacitinib is ulcerative colitis, and the daily upadacitinib dose of 30 mg may be considered for patients with refractory, severe, or extensive disease.

In some embodiments, the disease clinically approved for treatment with upadacitinib is Crohn's disease. In some embodiments, the disease clinically approved for treatment with upadacitinib is Crohn's disease, and the daily upadacitinib dose of 45 mg is reduced to 30 mg. In some embodiments, the disease clinically approved for treatment with upadacitinib is Crohn's disease, and the daily upadacitinib dose of 30 mg may be considered for patients with refractory, severe, or extensive disease.

In one aspect, the present disclosure provides a method for treating a disease clinically approved for treatment with upadacitinib in patients having severe renal impairment, the method comprising:
determining the recommended the daily upadacitinib dose for patients not having severe renal impairment, selected from 45 mg, 30 mg or 15 mg;
reducing the recommended daily upadacitinib dose from 45 mg to 30 mg during the time the patients have severe renal impairment;
reducing the recommended daily upadacitinib dose from 30 mg to 15 mg during the time the patients have severe renal impairment; and
maintaining the recommended daily upadacitinib dose of 15 mg during the time the patients have severe renal impairment.

In another aspect, the present disclosure provides a method for treating a disease clinically approved for treatment with upadacitinib in patients having severe renal impairment, the method comprising:
assessing whether the patients have severe renal impairment;
reducing a recommended daily upadacitinib dose of 45 mg to 30 mg during the time the patients have severe renal impairment;
reducing a recommended daily upadacitinib dose of 30 mg to 15 mg during the time the patients have severe renal impairment; and
maintaining a recommended daily upadacitinib dose of 15 mg during the time the patients have severe renal impairment.

In another aspect, the present disclosure provides a method for treating a disease clinically approved for treatment with upadacitinib in patients having severe renal impairment, the method comprising:
reducing the recommended daily upadacitinib dose for patients having severe renal impairment from 45 mg to a daily upadacitinib dose of 30 mg during the time the patients have severe renal impairment;
reducing the recommended daily upadacitinib dose for patients having severe renal impairment from 30 mg to a daily upadacitinib dose of 15 mg during the time the patients have severe renal impairment; and
maintaining the recommended daily upadacitinib dose of 15 mg during the time the patients have severe renal impairment.

In one aspect, the present disclosure provides a method for treating a disease clinically approved for treatment with upadacitinib in patients having severe renal impairment, the method comprising:
determining the recommended the daily upadacitinib dose for patients not having severe renal impairment, selected from 45 mg, 30 mg or 15 mg;
assessing whether the patients have severe renal impairment;
reducing the recommended daily upadacitinib dose from 45 mg to 30 mg during the time the patients have severe renal impairment;
reducing the recommended daily upadacitinib dose from 30 mg to 15 mg during the time the patients have severe renal impairment; and
maintaining the recommended daily upadacitinib dose of 15 mg during the time the patients have severe renal impairment.

In some embodiments, the daily upadacitinib dose for patients not having severe renal impairment is 45 mg, which is reduced to a daily upadacitinib dose of 30 mg during the time the patient is having severe renal impairment. In some embodiments, the daily upadacitinib dose of 30 mg during the time the patient is having severe renal impairment is administered without need for caution. In some embodiments, the patient is closely monitored for adverse reactions when administered the daily upadacitinib dose of 30 mg during the time the patient is having severe renal impairment. In some embodiments, the daily upadacitinib dose of 30 mg is administered as two 15 mg dosage forms.

In some embodiments, the daily upadacitinib dose for patients not having severe renal impairment is 30 mg, which is reduced to a daily upadacitinib dose of 15 mg during the time the patient is having severe renal impairment. In some embodiments, the daily upadacitinib dose of 15 mg during the time the patient is having severe renal impairment is administered without need for caution. In some embodiments, the patient is closely monitored for adverse reactions when administered the daily upadacitinib dose of 15 mg during the time the patient is having severe renal impairment.

In some embodiments, the daily upadacitinib dose for patients not having severe renal impairment is 15 mg, which is maintained during the time the patient is having severe renal impairment. In some embodiments, the daily upadacitinib dose of 15 mg during the time the patient is having severe renal impairment is administered without need for caution. In some embodiments, the patient is closely monitored for adverse reactions when administered the daily upadacitinib dose of 15 mg during the time the patient is having severe renal impairment.

In some embodiments, the daily upadacitinib dose for patients not having severe renal impairment is 15 mg, which is maintained during the time the patient is having severe renal impairment. In some embodiments, the daily upadacitinib dose of 15 mg during the time the patient is having severe renal impairment is administered without need for caution. In some embodiments, the patient is closely monitored for adverse reactions when administered the daily upadacitinib dose of 15 mg during the time the patient is having severe renal impairment.

In some embodiments, the disease clinically approved for treatment with upadacitinib is rheumatoid arthritis. In some embodiments, the disease clinically approved for treatment with upadacitinib is rheumatoid arthritis, and the daily upadacitinib dose of 15 mg is maintained. In some embodiments, the patient is a rheumatoid arthritis patient and the daily upadacitinib dose of 15 mg during the time the patient has severe renal impairment is administered without need for caution. In some embodiments, the disease clinically approved for treatment with upadacitinib is psoriatic arthritis. In some embodiments, the disease clinically approved for treatment with upadacitinib is psoriatic arthritis, and the daily upadacitinib dose of 15 mg is maintained.

In some embodiments, the disease clinically approved for treatment with upadacitinib is atopic dermatitis. In some embodiments, the disease clinically approved for treatment with upadacitinib is atopic dermatitis, and the daily upadacitinib dose of 15 mg is maintained. In some embodiments, the disease clinically approved for treatment with upadacitinib is atopic dermatitis, and the daily upadacitinib dose of 30 mg is reduced to 15 mg.

In some embodiments, the disease clinically approved for treatment with upadacitinib is ulcerative colitis. In some embodiments, the disease clinically approved for treatment with upadacitinib is ulcerative colitis, and the daily upadacitinib dose of 45 mg is reduced to 30 mg. In some embodiments, the disease clinically approved for treatment with upadacitinib is ulcerative colitis, and the daily upadacitinib dose of 30 mg may be considered for patients with refractory, severe, or extensive disease.

In some embodiments, the disease clinically approved for treatment with upadacitinib is Crohn's disease. In some embodiments, the disease clinically approved for treatment with upadacitinib is Crohn's disease, and the daily upadacitinib dose of 45 mg is reduced to 30 mg. In some embodiments, the disease clinically approved for treatment with upadacitinib is Crohn's disease, and the daily upadacitinib dose of 30 mg may be considered for patients with refractory, severe, or extensive disease.

In another aspect, the present disclosure provides a method for treating a disease clinically approved for treatment with upadacitinib in patients having severe renal impairment, the method comprising:
determining the recommended the daily upadacitinib dose for patients not having severe renal impairment, selected from 45 mg, 30 mg or 15 mg;
assessing whether the patients are having severe renal impairment;
administering a daily upadacitinib dose of 30 mg to patients having severe renal impairment during the time the patients are having severe renal impairment; and
administering a daily upadacitinib dose of 15 mg to patients having severe renal impairment during the time the patients are having severe renal impairment.

In a still further aspect, the present disclosure provides a method of treating a patient having severe renal impairment with upadacitinib, the method comprising:
reducing a recommended daily upadacitinib dose of 45 mg to 30 mg during the time the patient has severe renal impairment;
reducing a recommended daily upadacitinib dose of 30 mg to 15 mg during the time the patient has severe renal impairment; and
maintaining a recommended daily upadacitinib dose of 15 mg during the time the patient has severe renal impairment.

In another aspect, the present disclosure provides a method for treating a disease clinically approved for treatment with upadacitinib in a patient having severe renal impairment, the method comprising:
determining the recommended the daily upadacitinib dose when the patient is not having severe renal impairment, selected from 45 mg, 30 mg or 15 mg;
assessing whether the patient is having severe renal impairment; and
(a) reducing the recommended daily upadacitinib dose of 45 mg to a daily upadacitinib dose of 30 mg during the time the patient is having severe renal impairment,
(b) reducing the recommended daily upadacitinib dose of 30 mg to a daily upadacitinib dose of 15 mg during the time the patient is having severe renal impairment, or
(c) maintaining the recommended daily upadacitinib dose of 15 mg during the time the patient is having severe renal impairment.

In another aspect, the present disclosure provides a method for treating a disease clinically approved for treatment with upadacitinib in a patient having severe renal impairment, the method comprising:
determining the recommended the daily upadacitinib dose when the patient is not having severe renal impairment, selected from 45 mg, 30 mg or 15 mg;
assessing whether the patient is having severe renal impairment; and
(a) if the patient is having severe renal impairment and a daily upadacitinib dose of 45 mg, administering a daily upadacitinib dose of 30 mg during the time the patient is having severe renal impairment;
(b) if the patient is having severe renal impairment and a daily upadacitinib dose of 30 mg, administering a daily upadacitinib dose of 15 mg during the time the patient is having severe renal impairment; or
(c) if the patient is having severe renal impairment and a daily upadacitinib dose of 15 mg, administering a daily upadacitinib dose of 15 mg during the time the patient is having severe renal impairment.

In some embodiments, the daily upadacitinib dose for patients not having severe renal impairment is 45 mg, which is reduced to a daily upadacitinib dose of 30 mg during the time the patient is having severe renal impairment. In some embodiments, the daily upadacitinib dose of 30 mg during the time the patient is having severe renal impairment is administered without need for caution. In some embodiments, the patient is closely monitored for adverse reactions when administered the daily upadacitinib dose of 30 mg during the time the patient is having severe renal impairment. In some embodiments, the daily upadacitinib dose of 30 mg is administered as two 15 mg dosage forms.

In some embodiments, the daily upadacitinib dose for patients not having severe renal impairment is 30 mg, which is reduced to a daily upadacitinib dose of 15 mg during the time the patient is having severe renal impairment. In some embodiments, the daily upadacitinib dose of 15 mg during the time the patient is having severe renal impairment is administered without need for caution. In some embodiments, the patient is closely monitored for adverse reactions when administered the daily upadacitinib dose of 15 mg during the time the patient is having severe renal impairment.

In some embodiments, the daily upadacitinib dose for patients not having severe renal impairment is 15 mg, which is maintained during the time the patient is having severe renal impairment. In some embodiments, the daily upadacitinib dose of 15 mg during the time the patient is having severe renal impairment is administered without need for caution. In some embodiments, the patient is closely monitored for adverse reactions when administered the daily upadacitinib dose of 15 mg during the time the patient is having severe renal impairment.

In some embodiments, the daily upadacitinib dose for patients not having severe renal impairment is 15 mg, which is maintained during the time the patient is having severe renal impairment. In some embodiments, the daily upadacitinib dose of 15 mg during the time the patient is having severe renal impairment is administered without need for caution. In some embodiments, the patient is closely monitored for adverse reactions when administered the daily upadacitinib dose of 15 mg during the time the patient is having severe renal impairment.

In some embodiments, the disease clinically approved for treatment with upadacitinib is rheumatoid arthritis. In some embodiments, the disease clinically approved for treatment with upadacitinib is rheumatoid arthritis, and the daily upadacitinib dose of 15 mg is maintained. In some embodiments, the patient is a rheumatoid arthritis patient and the daily upadacitinib dose of 15 mg during the time the patient has severe renal impairment is administered without need for caution. In some embodiments, the disease clinically approved for treatment with upadacitinib is psoriatic arthritis. In some embodiments, the disease clinically approved for treatment with upadacitinib is psoriatic arthritis, and the daily upadacitinib dose of 15 mg is maintained.

In some embodiments, the disease clinically approved for treatment with upadacitinib is atopic dermatitis. In some embodiments, the disease clinically approved for treatment with upadacitinib is atopic dermatitis, and the daily upadacitinib dose of 15 mg is maintained. In some embodiments, the disease clinically approved for treatment with upadacitinib is atopic dermatitis, and the daily upadacitinib dose of 30 mg is reduced to 15 mg.

In some embodiments, the disease clinically approved for treatment with upadacitinib is ulcerative colitis. In some embodiments, the disease clinically approved for treatment with upadacitinib is ulcerative colitis, and the daily upadacitinib dose of 45 mg is reduced to 30 mg. In some embodiments, the disease clinically approved for treatment with upadacitinib is ulcerative colitis, and the daily upadacitinib dose of 30 mg may be considered for patients with refractory, severe, or extensive disease.

In some embodiments, the disease clinically approved for treatment with upadacitinib is Crohn's disease. In some embodiments, the disease clinically approved for treatment with upadacitinib is Crohn's disease, and the daily upadacitinib dose of 45 mg is reduced to 30 mg. In some embodiments, the disease clinically approved for treatment with upadacitinib is Crohn's disease, and the daily upadacitinib dose of 30 mg may be considered for patients with refractory, severe, or extensive disease.

In a still further aspect, the present disclosure provides a method of treating adult patients 65 years of age and older with upadacitinib, the method comprising:
maintaining a recommended daily upadacitinib dose of 45 mg;
reducing a recommended daily upadacitinib dose of 30 mg to 15 mg; and
maintaining a recommended daily upadacitinib dose of 15 mg.

In one aspect, the present disclosure provides a method for treating a disease clinically approved for treatment with upadacitinib in adult patients 65 years of age and older, the method comprising:
maintaining a recommended daily upadacitinib dose of 45 mg;
reducing a recommended daily upadacitinib dose of 30 mg to 15 mg; and
maintaining a recommended daily upadacitinib dose of 15 mg.

In one aspect, the present disclosure provides a method for treating a disease clinically approved for treatment with upadacitinib in adult patients 65 years of age and older, the method comprising:
maintaining a recommended daily upadacitinib dose of 45 mg;
reducing a recommended daily upadacitinib dose of 30 mg to 15 mg; and
maintaining a recommended daily upadacitinib dose of 15 mg.

In another aspect, the present disclosure provides a method for improving safety in treating a disease clinically approved for treatment with upadacitinib in adult patients 65 years of age and older, the method comprising:
maintaining a recommended daily upadacitinib dose of 45 mg;
reducing a recommended daily upadacitinib dose of 30 mg to 15 mg; and
maintaining a recommended daily upadacitinib dose of 15 mg.

In yet another aspect, the present disclosure provides a method of reducing an occurrence of or potential for adverse events in treating a disease clinically approved for treatment with upadacitinib in adult patients 65 years of age and older, the method comprising:
maintaining a recommended daily upadacitinib dose of 45 mg;
reducing a recommended daily upadacitinib dose of 30 mg to 15 mg; and
maintaining a recommended daily upadacitinib dose of 15 mg.

In a further aspect, the present disclosure provides a method of treating an FDA approved indication with upadacitinib in adult patients 65 years of age and older, the method comprising:
maintaining a recommended daily upadacitinib dose of 45 mg;
reducing a recommended daily upadacitinib dose of 30 mg to 15 mg; and
maintaining a recommended daily upadacitinib dose of 15 mg.

In some embodiments, the daily upadacitinib dose for adult patients not 65 years of age and older is 45 mg, which is maintained. In some embodiments, the daily upadacitinib dose of 45 mg in adult patients 65 years of age and older is administered without need for caution. In some embodiments, the patient is closely monitored for adverse reactions when administered the daily upadacitinib dose of 45 mg. In some embodiments, the daily upadacitinib dose of 45 mg is administered as three 15 mg dosage forms.

In some embodiments, the daily upadacitinib dose for adult patients not 65 years of age and older is 30 mg, which is reduced to a daily upadacitinib dose of 15 mg. In some embodiments, the daily upadacitinib dose of 15 mg is administered without need for caution. In some embodiments, the patient is closely monitored for adverse reactions when administered the daily upadacitinib dose of 15 mg.

In some embodiments, the daily upadacitinib dose for patients not 65 years of age and older is 15 mg, which is maintained. In some embodiments, the daily upadacitinib dose of 15 mg is administered without need for caution. In some embodiments, the patient is closely monitored for adverse reactions when administered the daily upadacitinib dose of 15 mg.

In some embodiments, the disease clinically approved for treatment with upadacitinib is rheumatoid arthritis. In some embodiments, the disease clinically approved for treatment with upadacitinib is rheumatoid arthritis, and the daily upadacitinib dose of 15 mg is maintained. In some embodiments, the patient is a rheumatoid arthritis patient and the daily upadacitinib dose of 15 mg is administered without need for caution. In some embodiments, the disease clinically approved for treatment with upadacitinib is psoriatic arthritis. In some embodiments, the disease clinically approved for treatment with upadacitinib is psoriatic arthritis, and the daily upadacitinib dose of 15 mg is maintained.

In some embodiments, the disease clinically approved for treatment with upadacitinib is atopic dermatitis. In some embodiments, the disease clinically approved for treatment with upadacitinib is atopic dermatitis, and the daily upadacitinib dose of 15 mg is maintained. In some embodiments, the disease clinically approved for treatment with upadacitinib is atopic dermatitis, and the daily upadacitinib dose of 30 mg is reduced to 15 mg.

In some embodiments, the disease clinically approved for treatment with upadacitinib is ulcerative colitis. In some embodiments, the disease clinically approved for treatment with upadacitinib is ulcerative colitis, and the daily upadacitinib dose of 45 mg is maintained as 45 mg. In some embodiments, the disease clinically approved for treatment with upadacitinib is ulcerative colitis, and the daily upadacitinib dose of 30 mg may be considered for patients with refractory, severe, or extensive disease.

In some embodiments, the disease clinically approved for treatment with upadacitinib is Crohn's disease. In some embodiments, the disease clinically approved for treatment with upadacitinib is Crohn's disease, and the daily upadacitinib dose of 45 mg is reduced to 30 mg. In some embodiments, the disease clinically approved for treatment with upadacitinib is Crohn's disease, and the daily upadacitinib dose of 30 mg may be considered for patients with refractory, severe, or extensive disease.

In one aspect, the present disclosure provides a method for treating a disease clinically approved for treatment with upadacitinib in adult patients 65 years of age and older, the method comprising:
maintaining a recommended daily upadacitinib dose of 45 mg;
reducing a recommended daily upadacitinib dose of 30 mg to 15 mg; and
maintaining a recommended daily upadacitinib dose of 15 mg.

In another aspect, the present disclosure provides a method for treating a disease clinically approved for treatment with upadacitinib in adult patients 65 years of age and older, the method comprising:
assessing whether the patients are 65 years of age and older;
maintaining a recommended daily upadacitinib dose of 45 mg;
reducing a recommended daily upadacitinib dose of 30 mg to 15 mg; and
maintaining a recommended daily upadacitinib dose of 15 mg.

In one aspect, the present disclosure provides a method for treating a disease clinically approved for treatment with upadacitinib in adult patients 65 years of age and older, the method comprising:
determining the recommended the daily upadacitinib dose for patients not 65 years of age and older, selected from 45 mg, 30 mg or 15 mg;
assessing whether the patients are 65 years of age and older;
maintaining a recommended daily upadacitinib dose of 45 mg;
reducing a recommended daily upadacitinib dose of 30 mg to 15 mg; and
maintaining a recommended daily upadacitinib dose of 15 mg.

In some embodiments, the daily upadacitinib dose for adult patients not 65 years of age and older is 30 mg, which is reduced to a daily upadacitinib dose of 15 mg. In some embodiments, the daily upadacitinib dose of 15 mg is administered without need for caution. In some embodiments, the patient is closely monitored for adverse reactions when administered the daily upadacitinib dose of 15 mg.

In some embodiments, the daily upadacitinib dose for patients not 65 years of age and older is 15 mg, which is maintained. In some embodiments, the daily upadacitinib dose of 15 mg is administered without need for caution. In some embodiments, the patient is closely monitored for adverse reactions when administered the daily upadacitinib dose of 15 mg.

In some embodiments, the disease clinically approved for treatment with upadacitinib is rheumatoid arthritis. In some embodiments, the disease clinically approved for treatment with upadacitinib is rheumatoid arthritis, and the daily upadacitinib dose of 15 mg is maintained. In some embodiments, the patient is a rheumatoid arthritis patient and the daily upadacitinib dose of 15 mg is administered without need for caution. In some embodiments, the disease clinically approved for treatment with upadacitinib is psoriatic arthritis. In some embodiments, the disease clinically approved for treatment with upadacitinib is psoriatic arthritis, and the daily upadacitinib dose of 15 mg is maintained.

In some embodiments, the disease clinically approved for treatment with upadacitinib is atopic dermatitis. In some embodiments, the disease clinically approved for treatment with upadacitinib is atopic dermatitis, and the daily upadacitinib dose of 15 mg is maintained. In some embodiments, the disease clinically approved for treatment with upadacitinib is atopic dermatitis, and the daily upadacitinib dose of 30 mg is reduced to 15 mg.

In some embodiments, the disease clinically approved for treatment with upadacitinib is ulcerative colitis. In some embodiments, the disease clinically approved for treatment with upadacitinib is ulcerative colitis, and the daily upadacitinib dose of 45 mg is reduced to 30 mg. In some embodiments, the disease clinically approved for treatment with upadacitinib is ulcerative colitis, and the daily upadacitinib dose of 30 mg may be considered for patients with refractory, severe, or extensive disease.

In some embodiments, the disease clinically approved for treatment with upadacitinib is Crohn's disease. In some embodiments, the disease clinically approved for treatment with upadacitinib is Crohn's disease, and the daily upadacitinib dose of 45 mg is reduced to 30 mg. In some embodiments, the disease clinically approved for treatment with upadacitinib is Crohn's disease, and the daily upadacitinib dose of 30 mg may be considered for patients with refractory, severe, or extensive disease.

In a still further aspect, the present disclosure provides a method of treating an adult patient 65 years of age and older, the method comprising:
maintaining a recommended daily upadacitinib dose of 45 mg;
reducing a recommended daily upadacitinib dose of 30 mg to 15 mg; or
maintaining a recommended daily upadacitinib dose of 15 mg.

In another aspect, the present disclosure provides a method for treating a disease clinically approved for treatment with upadacitinib in an adult patient 65 years of age and older, the method comprising:
determining the recommended the daily upadacitinib dose when the patient is not an adult patient 65 years of age and older, selected from 45 mg, 30 mg or 15 mg;
assessing whether the patient is an adult patient 65 years of age and older; and
(a) maintaining the recommended daily upadacitinib dose of 45 mg;
(b) reducing the recommended daily upadacitinib dose of 30 mg to 15 mg, or
(c) maintaining the recommended daily upadacitinib dose of 15 mg.

In some embodiments, the daily upadacitinib dose for adult patients not 65 years of age and older is 45 mg, which is maintained at 45 mg. In some embodiments, the daily upadacitinib dose of 45 mg is administered without need for caution. In some embodiments, the patient is closely monitored for adverse reactions when administered the daily upadacitinib dose of 45 mg. In some embodiments, the daily upadacitinib dose of 45 mg is administered as three 15 mg dosage forms.

In some embodiments, the daily upadacitinib dose for adult patients not 65 years of age and older is 30 mg, which is reduced to a daily upadacitinib dose of 15 mg. In some embodiments, the daily upadacitinib dose of 15 mg is administered without need for caution. In some embodiments, the patient is closely monitored for adverse reactions when administered the daily upadacitinib dose of 15 mg.

In some embodiments, the daily upadacitinib dose for adult patients not 65 years of age and older is 15 mg, which is maintained. In some embodiments, the daily upadacitinib dose of 15 mg is administered without need for caution. In some embodiments, the patient is closely monitored for adverse reactions when administered the daily upadacitinib dose of 15 mg.

In some embodiments, the daily upadacitinib dose for adult patients not 65 years of age and older is 15 mg, which is maintained. In some embodiments, the daily upadacitinib dose of 15 mg is administered without need for caution. In some embodiments, the patient is closely monitored for adverse reactions when administered the daily upadacitinib dose of 15 mg.

In some embodiments, the disease clinically approved for treatment with upadacitinib is rheumatoid arthritis. In some embodiments, the disease clinically approved for treatment with upadacitinib is rheumatoid arthritis, and the daily upadacitinib dose of 15 mg is maintained. In some embodiments, the patient is a rheumatoid arthritis patient and the daily upadacitinib dose of 15 mg is administered without need for caution. In some embodiments, the disease clinically approved for treatment with upadacitinib is psoriatic arthritis. In some embodiments, the disease clinically approved for treatment with upadacitinib is psoriatic arthritis, and the daily upadacitinib dose of 15 mg is maintained.

In some embodiments, the disease clinically approved for treatment with upadacitinib is atopic dermatitis. In some embodiments, the disease clinically approved for treatment with upadacitinib is atopic dermatitis, and the daily upadacitinib dose of 15 mg is maintained. In some embodiments, the disease clinically approved for treatment with upadacitinib is atopic dermatitis, and the daily upadacitinib dose of 30 mg is reduced to 15 mg.

In some embodiments, the disease clinically approved for treatment with upadacitinib is ulcerative colitis. In some embodiments, the disease clinically approved for treatment with upadacitinib is ulcerative colitis, and the daily upadacitinib dose of 45 mg is reduced to 30 mg. In some embodiments, the disease clinically approved for treatment with upadacitinib is ulcerative colitis, and the daily upadacitinib dose of 30 mg may be considered for patients with refractory, severe, or extensive disease.

In some embodiments, the disease clinically approved for treatment with upadacitinib is Crohn's disease. In some embodiments, the disease clinically approved for treatment with upadacitinib is Crohn's disease, and the daily upadacitinib dose of 45 mg is reduced to 30 mg. In some embodiments, the disease clinically approved for treatment with upadacitinib is Crohn's disease, and the daily upadacitinib dose of 30 mg may be considered for patients with refractory, severe, or extensive disease.

In a further aspect is provided a method of administering upadacitinib to a patient in need thereof, wherein the patient is receiving a strong CYP3A4 inhibitor for a period of time, the method comprising:
reducing a recommended daily upadacitinib dose of 45 mg to 30 mg during the period of time the patient is receiving the strong CYP3A4 inhibitor;
reducing a recommended daily upadacitinib dose of 30 mg to 15 mg during the period of time the patient is receiving the strong CYP3A4 inhibitor; or
administering a recommended daily upadacitinib dose of 15 mg without regard to whether the patient is receiving the strong CYP3A4 inhibitor.

In some embodiments, the recommended daily upadacitinib dose for patients not receiving a strong CYP3A4 inhibitor is 45 mg, which is reduced to a daily upadacitinib dose of 30 mg during the time the patient is receiving the strong CYP3A4 inhibitor. In some embodiments, the daily upadacitinib dose of 30 mg during the time the patient is receiving the strong CYP3A4 inhibitor is administered without need for caution. In some embodiments, the patient is closely monitored for adverse reactions when administered the daily upadacitinib dose of 30 mg during the time the patient is receiving the strong CYP3A4 inhibitor. In some embodiments, the daily upadacitinib dose of 30 mg is administered as two 15 mg dosage forms.

In some embodiments, the recommended daily upadacitinib dose for patients not receiving a strong CYP3A4 inhibitor is 30 mg, which is reduced to a daily upadacitinib dose of 15 mg during the time the patient is receiving the strong CYP3A4 inhibitor. In some embodiments, the daily upadacitinib dose of 15 mg during the time the patient is receiving the strong CYP3A4 inhibitor is administered without need for caution. In some embodiments, the patient is closely monitored for adverse reactions when administered the daily upadacitinib dose of 15 mg during the time the patient is receiving the strong CYP3A4 inhibitor.

In some embodiments, the recommended daily upadacitinib dose for patients not receiving a strong CYP3A4 inhibitor is 15 mg, which is maintained during the time the patient is receiving the strong CYP3A4 inhibitor. In some embodiments, the daily upadacitinib dose of 15 mg during the time the patient is receiving the strong CYP3A4 inhibitor is administered without need for caution. In some embodiments, the patient is closely monitored for adverse reactions when administered the daily upadacitinib dose of 15 mg during the time the patient is receiving the strong CYP3A4 inhibitor.

In some embodiments, the recommended daily upadacitinib dose for patients not receiving a strong CYP3A4 inhibitor is 15 mg, which is maintained during the time the patient is receiving the strong CYP3A4 inhibitor. In some embodiments, the daily upadacitinib dose of 15 mg during the time the patient is receiving the strong CYP3A4 inhibitor is administered without need for caution. In some embodiments, the patient is closely monitored for adverse reactions when administered the daily upadacitinib dose of 15 mg during the time the patient is receiving the strong CYP3A4 inhibitor.

In some embodiments, the disease clinically approved for treatment with upadacitinib is rheumatoid arthritis. In some embodiments, the disease clinically approved for treatment with upadacitinib is rheumatoid arthritis, and the daily upadacitinib dose of 15 mg is maintained. In some embodiments, the patient is a rheumatoid arthritis patient and the daily upadacitinib dose of 15 mg during the time the patient is receiving the strong CYP3A4 inhibitor is administered without need for caution. In some embodiments, the disease clinically approved for treatment with upadacitinib is psoriatic arthritis. In some embodiments, the disease clinically approved for treatment with upadacitinib is psoriatic arthritis, and the daily upadacitinib dose of 15 mg is maintained.

In some embodiments, the disease clinically approved for treatment with upadacitinib is atopic dermatitis. In some embodiments, the disease clinically approved for treatment with upadacitinib is atopic dermatitis, and the daily upadacitinib dose of 15 mg is maintained. In some embodiments, the disease clinically approved for treatment with upadacitinib is atopic dermatitis, and the daily upadacitinib dose of 30 mg is reduced to 15 mg.

In some embodiments, the disease clinically approved for treatment with upadacitinib is ulcerative colitis. In some embodiments, the disease clinically approved for treatment with upadacitinib is ulcerative colitis, and the daily upadacitinib dose of 45 mg is reduced to 30 mg. In some embodiments, the disease clinically approved for treatment with upadacitinib is ulcerative colitis, and the daily upadacitinib dose of 30 mg may be considered for patients with refractory, severe, or extensive disease.

In some embodiments, the disease clinically approved for treatment with upadacitinib is Crohn's disease. In some embodiments, the disease clinically approved for treatment with upadacitinib is Crohn's disease, and the daily upadacitinib dose of 45 mg is reduced to 30 mg. In some embodiments, the disease clinically approved for treatment with upadacitinib is Crohn's disease, and the daily upadacitinib dose of 30 mg may be considered for patients with refractory, severe, or extensive disease.

In a still further aspect is provided a method of administering a strong CYP3A4 inhibitor to a patient in need thereof for a period of time, wherein the patient is receiving upadacitinib, the method comprising:
reducing a recommended daily upadacitinib dose of 45 mg to 30 mg during the period of time the patient is receiving the strong CYP3A4 inhibitor;
reducing a recommended daily upadacitinib dose of 30 mg to 15 mg during the period of time the patient is receiving the strong CYP3A4 inhibitor; or
administering a recommended daily upadacitinib dose of 15 mg without regard to whether the patient is receiving the strong CYP3A4 inhibitor.

In some embodiments, the disease clinically approved for treatment with upadacitinib is Crohn's disease. In some embodiments, the disease clinically approved for treatment with upadacitinib is Crohn's disease, and the daily upadacitinib dose of 45 mg is reduced to 30 mg. In some embodiments, the disease clinically approved for treatment with upadacitinib is Crohn's disease, and the daily upadacitinib dose of 30 mg may be considered for patients with refractory, severe, or extensive disease.

In a further aspect is provided a method of administering upadacitinib to a patient in need thereof, wherein the patient is receiving a strong CYP3A4 inhibitor for a period of time, the method comprising:
reducing a recommended daily upadacitinib dose of 45 mg to 30 mg during the period of time the patient is receiving the strong CYP3A4 inhibitor;
reducing a recommended daily upadacitinib dose of 30 mg to 15 mg during the period of time the patient is receiving the strong CYP3A4 inhibitor; or
administering a recommended daily upadacitinib dose of 15 mg without regard to whether the patient is receiving the strong CYP3A4 inhibitor.

In some embodiments, the recommended daily upadacitinib dose for patients not receiving a strong CYP3A4 inhibitor is 45 mg, which is reduced to a daily upadacitinib dose of 30 mg during the time the patient is receiving the strong CYP3A4 inhibitor. In some embodiments, the daily upadacitinib dose of 30 mg during the time the patient is receiving the strong CYP3A4 inhibitor is administered without need for caution. In some embodiments, the patient is closely monitored for adverse reactions when administered the daily upadacitinib dose of 30 mg during the time the patient is receiving the strong CYP3A4 inhibitor. In some embodiments, the daily upadacitinib dose of 30 mg is administered as two 15 mg dosage forms.

In some embodiments, the recommended daily upadacitinib dose for patients not receiving a strong CYP3A4 inhibitor is 30 mg, which is reduced to a daily upadacitinib dose of 15 mg during the time the patient is receiving the strong CYP3A4 inhibitor. In some embodiments, the daily upadacitinib dose of 15 mg during the time the patient is receiving the strong CYP3A4 inhibitor is administered without need for caution. In some embodiments, the patient is closely monitored for adverse reactions when administered the daily upadacitinib dose of 15 mg during the time the patient is receiving the strong CYP3A4 inhibitor.

In some embodiments, the recommended daily upadacitinib dose for patients not receiving a strong CYP3A4 inhibitor is 15 mg, which is maintained during the time the patient is receiving the strong CYP3A4 inhibitor. In some embodiments, the daily upadacitinib dose of 15 mg during the time the patient is receiving the strong CYP3A4 inhibitor is administered without need for caution. In some embodiments, the patient is closely monitored for adverse reactions when administered the daily upadacitinib dose of 15 mg during the time the patient is receiving the strong CYP3A4 inhibitor.

In some embodiments, the recommended daily upadacitinib dose for patients not receiving a strong CYP3A4 inhibitor is 15 mg, which is maintained during the time the patient is receiving the strong CYP3A4 inhibitor. In some embodiments, the daily upadacitinib dose of 15 mg during the time the patient is receiving the strong CYP3A4 inhibitor is administered without need for caution. In some embodiments, the patient is closely monitored for adverse reactions when administered the daily upadacitinib dose of 15 mg during the time the patient is receiving the strong CYP3A4 inhibitor.

In some embodiments, the disease clinically approved for treatment with upadacitinib is rheumatoid arthritis. In some embodiments, the disease clinically approved for treatment with upadacitinib is rheumatoid arthritis, and the daily upadacitinib dose of 15 mg is maintained. In some embodiments, the patient is a rheumatoid arthritis patient and the daily upadacitinib dose of 15 mg during the time the patient is receiving the strong CYP3A4 inhibitor is administered without need for caution. In some embodiments, the disease clinically approved for treatment with upadacitinib is psoriatic arthritis. In some embodiments, the disease clinically approved for treatment with upadacitinib is psoriatic arthritis, and the daily upadacitinib dose of 15 mg is maintained.

In some embodiments, the disease clinically approved for treatment with upadacitinib is atopic dermatitis. In some embodiments, the disease clinically approved for treatment with upadacitinib is atopic dermatitis, and the daily upadacitinib dose of 15 mg is maintained. In some embodiments, the disease clinically approved for treatment with upadacitinib is atopic dermatitis, and the daily upadacitinib dose of 30 mg is reduced to 15 mg.

In some embodiments, the disease clinically approved for treatment with upadacitinib is ulcerative colitis. In some embodiments, the disease clinically approved for treatment with upadacitinib is ulcerative colitis, and the daily upadacitinib dose of 45 mg is reduced to 30 mg. In some embodiments, the disease clinically approved for treatment with upadacitinib is ulcerative colitis, and the daily upadacitinib dose of 30 mg may be considered for patients with refractory, severe, or extensive disease.

In some embodiments, the disease clinically approved for treatment with upadacitinib is Crohn's disease. In some embodiments, the disease clinically approved for treatment with upadacitinib is Crohn's disease, and the daily upadacitinib dose of 45 mg is reduced to 30 mg. In some embodiments, the disease clinically approved for treatment with upadacitinib is Crohn's disease, and the daily upadacitinib dose of 30 mg may be considered for patients with refractory, severe, or extensive disease.

### BRIEF DESCRIPTION OF THE DRAWINGS

FIG. 1 is a graph showing the percent of patients achieving an American College of Rheumatology ≥20% improvement (ACR20) in rheumatoid arthritis when treated with upadacitinib 15 mg plus methotrexate or with methotrexate alone.

FIG. 2 is a graph showing the percent of patients achieving an American College of Rheumatology ≥20% improvement (ACR20) in psoriatic arthritis when treated with upadacitinib 15 mg or placebo.

FIG. 3A is a graph showing the proportion of patients with atopic dermatitis achieving an improvement of 4 point or greater in the Worst Pruritis NRS when treated with upadacitinib 15 mg, 30 mg, or placebo in trial AD-1.

FIG. 3B is a graph showing the proportion of patients with atopic dermatitis achieving an improvement of 4 point or greater in the Worst Pruritis NRS when treated with upadacitinib 15 mg, 30 mg, or placebo in trial AD-2.

FIG. 4 is a graph showing the proportion of patients with atopic dermatitis achieving an improvement of 4 point or greater in the Worst Pruritis NRS when treated with upadacitinib 15 mg+topical corticosteroids (TCS), 30 mg+TCS, or placebo+TCS.

### DETAILED DESCRIPTION

This written description uses examples to disclose the invention and also to enable any person skilled in the art to practice the invention, including making and using any of the disclosed compositions, and performing any of the disclosed methods or processes. The patentable scope of the invention is defined by the claims, and may include other examples that occur to those skilled in the art. Such other examples are intended to be within the scope of the claims if they have elements that do not differ from the literal language of the claims, or if they include equivalent elements.

### Definitions

Section headings as used in this section and the entire disclosure are not intended to be limiting.

Where a numeric range is recited, each intervening number within the range is explicitly contemplated with the same degree of precision. For example, for the range 6 to 9, the numbers 7 and 8 are contemplated in addition to 6 and 9, and for the range 6.0 to 7.0, the numbers 6.0, 6.1, 6.2, 6.3, 6.4, 6.5, 6.6, 6.7, 6.8, 6.9 and 7.0 are explicitly contemplated. In the same manner, all recited ratios also include all sub-ratios falling within the broader ratio.

The singular forms “a,” “an” and “the” include plural referents unless the context clearly dictates otherwise.

The term “about” generally refers to a range of numbers that one of skill in the art would consider equivalent to the recited value (i.e., having the same function or result). In many instances, the term “about” may include numbers that are rounded to the nearest significant figure.

Unless the context requires otherwise, the terms “comprise,” “comprises,” and “comprising” are used on the basis and clear understanding that they are to be interpreted inclusively, rather than exclusively, and that Applicant intends each of those words to be so interpreted in construing this patent, including the claims below.

The term “AUC” refers to the area under the curve. AUC is the definite integral of a curve that describes the variation of a drug concentration in blood plasma as a function of time.

The term “Cₘₐₓ” refers to the plasma concentration of the referent drug at Tₘₐₓ, expressed herein as ng/mL, produced by the oral ingestion of a single dose, or indicated number of doses, of the dosage form or pharmaceutical composition, such as the dosage forms and compositions of the present disclosure. Unless specifically indicated, Cₘₐₓ refers to the overall maximum observed concentration.

The term “daily dose of upadacitinib” as used herein refers to a dose to be administered once daily to a patient in an extended-release form. Recommended daily doses of upadacitinib may vary as disclosed herein, and may be 15 mg, 30 mg, or 45 mg. For avoidance of doubt, dosages of 45 mg and 30 mg are intended to encompass multiples of 15 mg (e.g., dosages of 3 ×15 mg or 2 ×15 mg), administered together or administered sequentially and separated by a period of time.

The terms “treating”, “treatment”, and “therapy” and the like, as used herein, are meant to include therapeutic as well as prophylactic, or suppressive measures for a disease or disorder leading to any clinical desirable or beneficial effect, including but not limited to alleviation or relief of one or more symptoms, regression, slowing or cessation of progression of the disease or disorder. Thus, for example, the term treatment includes the administration of an agent prior to or following the onset of a symptom of a disease or disorder thereby preventing or removing one or more signs of the disease or disorder. As another example, the term includes the administration of an agent after clinical manifestation of the disease to combat the symptoms of the disease. Further, administration of an agent after onset and after clinical symptoms have developed where administration affects clinical parameters of the disease or disorder, such as the degree of tissue injury or the amount or extent of metastasis, whether or not the treatment leads to amelioration of the disease, comprises “treatment” or “therapy” as used herein. Moreover, as long as the compositions of the disclosure either alone or in combination with another therapeutic agent alleviate or ameliorate at least one symptom of a disorder being treated as compared to that symptom in the absence of use of the JAK1 inhibitor composition, the result should be considered an effective treatment of the underlying disorder regardless of whether all the symptoms of the disorder are alleviated or not.

### Upadacitinib Recommended Dosage

In some embodiments, the methods disclosed herein comprise determining the recommended daily dose of upadacitinib. The recommended dosage of upadacitinib may vary according to a number of factors, including, but not limited to, the specific disease to be treated, induction versus maintenance stage, patient age, comorbidities, and concurrent usage of certain medications as described herein below. Unless otherwise indicated, the recommended dosage is the daily dosage recommended in the absence of any complicating factor (i.e., renal or hepatic impairment or concurrent administration of drugs interacting with upadacitinib metabolism).

In some embodiments, the methods disclosed herein comprise determining the recommended daily dose of upadacitinib in patients not receiving a strong CYP3A4 inhibitor, or not having severe renal impairment. In some embodiments, the daily dose in patients not receiving a strong CYP3A4 inhibitor, or not having severe renal impairment is 45 mg, 30 mg, or 15 mg.

The recommended dosage of upadacitinib in patients with rheumatoid arthritis is 15 mg once daily.

The recommended dosage of upadacitinib in patients with psoriatic arthritis is 15 mg once daily.

The recommended dosage of upadacitinib in patients with atopic dermatitis may vary with age and/or response to treatment, and is generally the lowest effective dose needed to maintain response. The recommended dosage of upadacitinib in pediatric patients 12 years of age and older weighing at least 40 kg is 15 mg once daily to initiate treatment. If an adequate response is not achieved, the physician may consider increasing the dosage to 30 mg once daily. If an adequate response is not achieved with the 30 mg dose, treatment should be discontinued. The recommended dosage of upadacitinib in adult patients less than 65 years of age is 15 mg once daily to initiate treatment. If an adequate response is not achieved, the physician may consider increasing the dosage to 30 mg once daily. In some embodiments, the dosage is administered in a single 30 mg dosage form. In some embodiments, the dosage is administered as two 15 mg dosage forms. If an adequate response is not achieved with the 30 mg dose, treatment should be discontinued. The recommended dosage of upadacitinib in adult patients 65 years of age and older is 15 mg once daily

The recommended dosage of upadacitinib in patients with ulcerative colitis is an induction dose of 45 mg once daily for 8 weeks. In some embodiments, the dosage is administered in a single 45 mg dosage form. In some embodiments, the dosage is administered as three 15 mg dosage forms. The recommended maintenance dosage of upadacitinib in patients with ulcerative colitis is 15 mg once daily. A maintenance dosage of 30 mg once daily may be considered for patients with refractory, severe, or extensive disease. In some embodiments, the dosage is administered in a single 30 mg dosage form. In some embodiments, the dosage is administered as two 15 mg dosage forms. If an adequate response is not achieved with the 30 mg dose, treatment should be discontinued. The lowest effective dosage needed to maintain response should be used.

The recommended dosage in patients with renal or hepatic impairment may vary from the recommended dosage in the absence of renal or hepatic impairment, and is described further herein below. Modifications to the recommended dosage due to drug interactions may be required, and are described further herein below.

### Upadacitinib Metabolism

The potential of upadacitinib to act as perpetrator in drug-drug interactions was thoroughly investigated in vitro. In vitro, upadacitinib was a substrate for CYP3A4, CYP2D6, P-pg and BCRP. Upadacitinib was mainly metabolized by CYP3A4 and to a lesser extent by CYP2D6. Upadacitinib was not a substrate for CYP1A2, 2B6, 2C8, 2C9, 2C18, 2C19, 2J2, FMO1, FMO3, OATP1B1, OATP1B3 or OCT1 in vitro. Upadacitinib was an inducer of CYP3A4 and CYP2B6 in vitro while the results for CYP1A2 were borderline with only a minor concentration dependence.

In vivo, the contribution of CYP2D6 is expected to be minor given the comparability of upadacitinib CL/F in extensive and poor metabolizers in the Phase 1 and Phase 2 population (popPK analysis). Given the comparability of upadacitinib CL/F between subjects with extensive and poor metabolizer phenotypes for CYP2D6, concomitant medications that are strong inhibitors of CYP2D6 are expected to have no effect on upadacitinib plasma exposures.

There was no in vivo relevant inhibition by upadacitinib on any of the enzymes (CYP1A2, 2B6, 2C8, 2C9, 2C19, 2D6, 3A4) or transporters evaluated in vitro (P-gp, BCRP, BSEP, OATP1B1, OATB1B3, OCT1, OCT2, OAT1, OATS, MATE1, MATE2K).

### Upadacitinib Route of Elimination

Upadacitinib is eliminated both by the renal and the hepatic route. Following single dose administration of [¹⁴C]-radiolabeled upadacitinib immediate-release solution, upadacitinib was eliminated predominantly as the unchanged parent substance in urine (24%) and feces (31%). This fraction may originate either from absorbed and biliary secreted upadacitinib or of unabsorbed drug.

Renal clearance indicates some involvement of renal transporters in the excretion. Renal secretion is however not estimated to contribute to more than 25% of the elimination. The effect of renal impairment on upadacitinib PK was evaluated in the Phase 1 Study (n=6, mild renal impairment; n=6, moderate renal impairment; n=6, severe renal impairment; n=6, normal renal function). Results from the study demonstrated that upadacitinib exposure (AUC_{inf}) central values were 18%, 33%, and 44% higher in subjects with mild, moderate, and severe renal impairment, respectively, compared to subjects with normal renal function. This is consistent with an approximately 25% contribution of the renal route to upadacitinib elimination. As used herein, reference to “severe renal impairment” means a creatinine clearance of less than 30 mL/min. Age was evaluated as a continuous covariate as well as a discrete covariate (adolescents [weighing ≥40 kg] versus adults) on upadacitinib clearance (CL/F) and volume of distribution (Vc/F) in the population pharmacokinetic analyses and was not found to be statistically significant.

In a study with subjects with mild hepatic impairment (Child-Pugh [CI]-A, N=6), moderate hepatic impairment (CP-B, N=6), and with normal hepatic function (N=6), the effect of mild and moderate hepatic impairment on upadacitinib pharmacokinetics was modest. Upadacitinib AUC increased by 28 and 24% in subjects with mild and moderate hepatic impairment respectively compared to normal subjects.

The effect of different intrinsic factors has been evaluated in dedicated PK studies in renal and hepatic impairment and in Asian subjects, and by population pharmacokinetic analysis from the phase 2/3 studies. The population pharmacokinetic analysis indicated no relevant effects of gender, race, age or weight on upadacitinib pharmacokinetic parameters. There are limited data in patients aged 75 years and older and there is no pharmacokinetic data in children and adolescents.

### Potential for Upadacitinib-Drug Interactions

The effect of repeated doses of 30 mg upadacitinib on the pharmacokinetics of specific substrates of different CYP enzymes (CYP1A2, 3A, 2D6, 2C9 and 2C19) was evaluated in an in vivo cocktail study, and the potential for drug-drug interactions between upadacitinib and commonly used concomitant medications as well as probe substrates for CYP450 enzymes was characterized in several Phase 1 studies.

In vivo, the exposure of oral midazolam, a sensitive CYP3A4 substrate, was decreased by 26%, indicating that upadacitinib (30 mg dose) is a weak inducer of the PXR-mediated metabolism by CYP3A4. The effect is expected to be lower with the clinical dose of 15 mg.

The effect of the strong CYP3A/P-gp inhibitor ketoconazole, 400 mg QD, on upadacitinib administered 30 mg QD as the IR formulation was weak to moderate; upadacitinib AUC and Cmax increased 1.8- and 1.7-fold respectively, compared to administration of upadacitinib alone. There was no effect on half-life indicating an effect mainly on pre-systemic metabolism or P-gp. The effect of moderate CYP3A4/P-gp inhibitors on upadacitinib exposure is not expected to be clinically relevant.

Following multiple doses of rifampicin, a strong CYP3A/P-gp inducer, 600 mg QD for 8 days, upadacitinib AUC and Cmax decreased by 60% and 50% respectively. In summary, strong inducers of CYP3A (e.g., rifampin) reduce upadacitinib plasma exposures by approximately half. Strong CYP3A inhibitors (e.g., ketoconazole) increase upadacitinib AUC by 75% and maximum observed concentration (Cmax) by 70%.

Further results of these studies are summarized as follows:
There was no effect on upadacitinib exposure following co-administration with methotrexate.
Upadacitinib 30 mg QD decreased rosuvastatin AUC by 33% and atorvastatin AUC by 23% while its metabolite ortho-hydroxyatorvastatin AUC remained unchanged.
There was no inducing effect of upadacitinib on ethinylestradiol/levonorgestrel following multiple doses of upadacitinib ER 30 mg.
There was no relevant effect on bupropion AUC or Cmax and hence, no indication of upadacitinib being an inducer of CYP2B6 in vivo.
There was no relevant effect on the plasma exposure of other PXR regulated enzymes (CYP2C9 and 2C19) or enzymes regulated by the Ah-receptor (CYP1A2) Upadacitinib has no clinically relevant effects on plasma exposures of MTX, ethinylestradiol, levonorgestrel, statins, or drugs that are substrates for metabolism by CYP1A2, CYP2B6, CYP2D6, CYP2C19, CYP2C9, or CYP3A.
Concomitant administration of strong CYP2D6 inhibitors, OATP1B inhibitors, MTX, pH modifying medications, or statins has no effect on upadacitinib plasma exposures.
Overall, in atopic dermatitis studies with upadacitinib, plasma exposures were similar between adult and adolescent subjects

As described herein above, strong CYP3A4 inhibitors (e.g., ketoconazole) increase upadacitinib AUC by 75% and maximum observed concentration (Cmax) by 70%. As used herein, the term “strong CYP3A4 inhibitors” refers to drugs that increase the AUC of sensitive index CYP3A4 substrates (e.g., midazolam) by an amount of ≥5-fold. Examples of classes of drugs which include strong CYP3M4 inhibitors include certain antibiotics, antifungals, antidepressants, and antivirals. Examples of antibiotics which are strong CYP3A4 inhibitors include, but are not limited to, clarithromycin, telithromycin, and troleandomycin. Examples of antifungals which are strong CYP3A4 inhibitors include, but are not limited to, itraconazole, ketoconazole, posaconazole, and voriconazole. Examples of antivirals which are strong CYP3A4 inhibitors are the class of protease inhibitors which include, but are not limited to, atazanavir, hoceprevir, cobicistat, danoprevir, darunavir, dasabuvir, elvitegravir, indinavir, lopinavir, nelfinavir, ombitasvir, paritaprevir, ritonavir, saquinavir, telaprevir, and ipranavir. An example of an antidepressant which is a strong inhibitor of CYP3A4 is nefazodone. Grapefruit juice, depending on the source and preparation, may be a strong CYP3A4 inhibitor.

### Upadacitinib Dosing Modifications Due to Drug Interactions

In some embodiments, the methods disclosed herein comprise assessing whether a patient is receiving a strong CYP3A4 inhibitor. When both upadacitinib and a strong CYP3A4 inhibitor are administered to a patient, the recommended dosage of upadacitinib may vary from the recommended dosage of upadacitinib in patients not having severe renal impairment. The modification to the dosage is dependent on the disease being treated by the upadacitinib and the dosage recommended for treatment of the specific disease in the absence of administration of the strong CYP3A4 inhibitor. In some embodiments, the daily dose of upadacitinib in a patient receiving a strong CYP3A4 inhibitor is reduced relative to the daily dose of upadacitinib in the absence of the strong CYP3A4 inhibitor. In some embodiments, the daily dose of upadacitinib in a patient receiving a strong CYP3A4 inhibitor is maintained relative to the daily dose of upadacitinib in the absence of the strong CYP3A4 inhibitor (i.e., no dosage adjustment is necessary). Accordingly, in some embodiments, the methods disclosed herein comprise reducing or maintaining a dose of upadacitinib. In some embodiments, the methods disclosed herein comprise, in a patient receiving a strong CYP3A4 inhibitor, reducing a recommended daily upadacitinib dose of 45 mg to 30 mg, or from 30 mg to 15 mg. In some embodiments, the methods disclosed herein comprise, in a patient receiving a strong CYP3A4 inhibitor, maintaining a recommended daily upadacitinib dose of 15 mg.

For patients administered upadacitinib for treatment of rheumatoid arthritis or psoriatic arthritis in the absence of administration of a strong CYP3A4 inhibitor, the recommended daily dosage is 15 mg. For patients administered upadacitinib for treatment of rheumatoid arthritis or psoriatic arthritis, when receiving a strong CYP3A4 inhibitor, the recommended dosage is 15 mg once daily.

In some embodiments, for patients administered upadacitinib for treatment of atopic dermatitis in the absence of administration of a strong CYP3A4 inhibitor, the recommended daily dosage is 15 mg once daily. For patients administered upadacitinib for treatment of atopic dermatitis, when receiving a strong CYP3A4 inhibitor, the recommended dosage is 15 mg once daily. Accordingly, in some embodiments, the methods disclosed herein comprise maintaining the recommended daily dose of upadacitinib of 15 mg.

In some embodiments, for patients administered upadacitinib for treatment of atopic dermatitis in the absence of administration of a strong CYP3A4 inhibitor, the recommended daily dosage is 30 mg once daily. For patients administered upadacitinib for treatment of atopic dermatitis, when receiving a strong CYP3A4 inhibitor, the recommended dosage is 15 mg once daily. Accordingly, in some embodiments, the methods disclosed herein comprise reducing the recommended daily dose of upadacitinib from 30 mg to 15 mg.

For patients administered upadacitinib for treatment of ulcerative colitis in the absence of administration of a strong CYP3A4 inhibitor, the recommended daily dosage is an induction dose of 45 mg once a day for 8 weeks, followed by a maintenance dose of 15 mg once daily, or 30 mg once daily for patients with refractory, severe or extensive disease. For patients administered upadacitinib for treatment of ulcerative colitis, when receiving a strong CYP3A4 inhibitor, the recommended dosage is 30 mg once daily for induction, and 15 mg daily for maintenance. Accordingly, in some embodiments, the methods disclosed herein comprise reducing the recommended daily dose induction dose of upadacitinib from 45 mg to 30 mg. In some embodiments, the methods disclosed herein comprise reducing the recommended daily dose maintenance dose of upadacitinib from 30 mg to 15 mg. In some embodiments, the methods disclosed herein comprise maintaining the recommended daily dose maintenance dose of upadacitinib of 15 mg.

In some embodiments, a daily dose of 30 mg is maintained, and the dose of 30 mg is administered without need for caution. In some embodiments, a reduced daily dose of 30 mg is administered without need for caution.

In some embodiments, a daily dose of 30 mg is maintained, and the patient is closely monitored for adverse reactions. In some embodiments, a reduced daily dose of 30 mg is administered, and the patient is closely monitored for adverse reactions.

In some embodiments, a daily dose of 15 mg is maintained, and the dose of 15 mg is administered without need for caution. In some embodiments, a reduced daily dose of 15 mg is administered without need for caution.

In some embodiments, a daily dose of 15 mg is maintained, and the patient is closely monitored for adverse reactions. In some embodiments, a reduced daily dose of 15 mg is administered, and the patient is closely monitored for adverse reactions.

In any of the foregoing embodiments, it is contemplated herein that dosages of 30 mg may be administered as two 15 mg dosage forms.

Upadacitinib Dosing in Subjects with Renal Impairment

In some embodiments, the methods disclosed herein comprise assessing whether a patient has renal impairment. When upadacitinib is administered to a patient having renal impairment, the recommended dosage of upadacitinib may vary from the recommended dosage in the absence of renal impairment. The modification to the dosage is dependent on the disease being treated by the upadacitinib, the dosage recommended for treatment of the specific disease in the absence of renal impairment, and the extent of renal impairment. Accordingly, in some embodiments, the methods disclosed herein comprise reducing or maintaining a dose of upadacitinib. In some embodiments, the methods disclosed herein comprise, in a patient having renal impairment, reducing a recommended daily upadacitinib dose of 45 mg to 30 mg, or from 30 mg to 15 mg. In some embodiments, the methods disclosed herein comprise, in a patient having renal impairment, maintaining the recommended daily upadacitinib dose of 15 mg.

For patients administered upadacitinib for treatment of rheumatoid arthritis or psoriatic arthritis in the absence of any renal impairment, the recommended daily dosage is 15 mg. For patients administered upadacitinib for treatment of rheumatoid arthritis or psoriatic arthritis, the patient having mild, moderate, or severe renal impairment, the recommended dosage is 15 mg once daily. Accordingly, in some embodiments, the methods disclosed herein comprise maintaining the recommended daily dose of upadacitinib of 15 mg.

In some embodiments, for patients administered upadacitinib for treatment of atopic dermatitis in the absence of any renal impairment, the recommended daily dosage is 15 mg. In some embodiments, for patients administered upadacitinib for treatment of atopic dermatitis in the absence of any renal impairment, the recommended daily dosage is 30 mg once daily.

For patients administered upadacitinib for treatment of atopic dermatitis, the patient also having mild or moderate renal impairment (estimated glomerular filtration rate (eGFR) ≥30 mL/min/1.73 m²), the recommended dosage is 15 mg once daily. Accordingly, in some embodiments, the methods disclosed herein comprise maintaining the recommended daily dose of upadacitinib of 15 mg. For patients administered upadacitinib for treatment of atopic dermatitis, the patient also having severe renal impairment (eGFR <30 mL/min/1.73 m²), the recommended dosage is 15 mg once daily. Accordingly, in some embodiments, the methods disclosed herein comprise maintaining the recommended daily dose of upadacitinib of 15 mg. In some embodiments, the methods disclosed herein comprise reducing the recommended daily dose of 30 mg upadacitinib to 15 mg. For patients with atopic dermatitis and end stage renal disease (eGFR <15 mL/min/1.73 m²), use of upadacitinib is not recommended.

For patients administered upadacitinib for treatment of ulcerative colitis in the absence of any renal impairment, the recommended daily dosage is an induction dose of 45 mg once a day for 8 weeks, followed by a maintenance dose of 15 mg once daily, or 30 mg once daily for patients with refractory, severe or extensive disease. For patients administered upadacitinib for treatment of ulcerative colitis, the patient also having mild or moderate renal impairment (eGFR ≥30 mL/min/1.73 m²), the recommended dosage is 45 mg once daily for induction, and 15 or 30 mg daily for maintenance. Accordingly, in some embodiments, the methods disclosed herein comprise maintaining the recommended daily dose induction and maintenance dose of upadacitinib (45 mg and either 15 or 30 mg, respectively).

For patients administered upadacitinib for treatment of ulcerative colitis, the patient also having severe renal impairment (eGFR <30 mL/min/1.73 m²), the recommended induction dosage is 30 mg once daily for 8 weeks, and the recommended maintenance dose is 15 mg once daily. Accordingly, in some embodiments, the methods disclosed herein comprise reducing the recommended daily induction dose of upadacitinib of 45 mg to 30 mg. In some embodiments, the methods disclosed herein comprise reducing the recommended daily maintenance dose of 30 mg upadacitinib to 15 mg. In some embodiments, the methods disclosed herein comprise maintaining the recommended daily maintenance dose of 15 mg of upadacitinib. For patients with ulcerative colitis and end stage renal disease (eGFR <15 mL/min/1.73 m²), use of upadacitinib is not recommended.

In some embodiments, a daily dose of 30 mg is maintained, and the dose of 30 mg is administered without need for caution. In some embodiments, a reduced daily dose of 30 mg is administered without need for caution.

In some embodiments, a daily dose of 30 mg is maintained, and the patient is closely monitored for adverse reactions. In some embodiments, a reduced daily dose of 30 mg is administered, and the patient is closely monitored for adverse reactions.

In some embodiments, a daily dose of 15 mg is maintained, and the dose of 15 mg is administered without need for caution. In some embodiments, a reduced daily dose of 15 mg is administered without need for caution.

In some embodiments, a daily dose of 15 mg is maintained, and the patient is closely monitored for adverse reactions. In some embodiments, a reduced daily dose of 15 mg is administered, and the patient is closely monitored for adverse reactions.

In any of the foregoing embodiments, it is contemplated herein that dosages of 30 mg may be administered as two 15 mg dosage forms.

Upadacitinib Dosing in Subjects with Hepatic Impairment

When upadacitinib is administered to a patient having hepatic impairment, the recommended dosage of upadacitinib may vary from the recommended dosage in the absence of hepatic impairment. The modification to the dosage is dependent on the disease being treated by the upadacitinib, the dosage recommended for treatment of the specific disease in the absence of hepatic impairment, and the extent of hepatic impairment. Upadacitinib is not recommended for use in patients with severe hepatic impairment (Child-Pugh C).

For patients administered upadacitinib for treatment of rheumatoid arthritis or psoriatic arthritis in the absence of any hepatic impairment, the recommended daily dosage is 15 mg. For patients administered upadacitinib for treatment of rheumatoid arthritis or psoriatic arthritis, the patient having mild or moderate hepatic impairment, the recommended dosage is 15 mg once daily. Accordingly, in some embodiments, the methods disclosed herein comprise maintaining the recommended daily dose of upadacitinib of 15 mg.

In some embodiments, for patients administered upadacitinib for treatment of atopic dermatitis in the absence of any hepatic impairment, the recommended daily dosage is 15 mg. In some embodiments, for patients administered upadacitinib for treatment of atopic dermatitis in the absence of any hepatic impairment, the recommended daily dosage is 30 mg once daily. For patients administered upadacitinib for treatment of atopic dermatitis, the patient also having mild or moderate hepatic impairment, the recommended dosage is 15 mg once daily. Accordingly, in some embodiments, the methods disclosed herein comprise maintaining the recommended daily dose of upadacitinib of 15 mg. In some embodiments, the methods disclosed herein comprise maintaining the recommended daily dose of upadacitinib of 30 mg.

For patients administered upadacitinib for treatment of ulcerative colitis in the absence of any hepatic impairment, the recommended daily dosage is an induction dose of 45 mg once a day for 8 weeks, followed by a maintenance dose of 15 mg once daily, or 30 mg once daily for patients with refractory, severe or extensive disease. For patients administered upadacitinib for treatment of ulcerative colitis, the patient also having mild or moderate hepatic impairment, the recommended induction dosage is 30 mg once daily for 8 weeks, and the recommended maintenance dose is 15 mg once daily. Accordingly, in some embodiments, the methods disclosed herein comprise reducing the recommended daily induction dose of upadacitinib of 45 mg to 30 mg. In some embodiments, the methods disclosed herein comprise reducing the recommended daily maintenance dose of 30 mg upadacitinib to 15 mg. In some embodiments, the methods disclosed herein comprise maintaining the recommended daily maintenance dose of 15 mg of upadacitinib.

In some embodiments, a daily dose of 30 mg is maintained, and the dose of 30 mg is administered without need for caution. In some embodiments, a reduced daily dose of 30 mg is administered without need for caution.

In some embodiments, a daily dose of 30 mg is maintained, and the patient is closely monitored for adverse reactions. In some embodiments, a reduced daily dose of 30 mg is administered, and the patient is closely monitored for adverse reactions.

In some embodiments, a daily dose of 15 mg is maintained, and the dose of 15 mg is administered without need for caution. In some embodiments, a reduced daily dose of 15 mg is administered without need for caution.

In some embodiments, a daily dose of 15 mg is maintained, and the patient is closely monitored for adverse reactions. In some embodiments, a reduced daily dose of 15 mg is administered, and the patient is closely monitored for adverse reactions.

In any of the foregoing embodiments, it is contemplated herein that dosages of 30 mg may be administered as two 15 mg dosage forms.

### EXEMPLIFICATION

Examples 1 and 2 provide prescribing information for RINVOQ® (upadacitinib).

### Example 1. RINVOQ Prescribing Information

### Highlights of Prescribing Information

These highlights do not include all the information needed to use RINVOQ® safely and effectively. See full prescribing information for RINVOQ®. RINVOQ® (upadacitinib) extended-release tablets, for oral use Initial U.S. Approval: 2019. WARNING: SERIOUS INFECTIONS, MORTALITY, MALIGNANCY, MAJOR ADVERSE CARDIOVASCULAR EVENTS (MACE), AND THROMBOSIS. See full prescribing information for complete boxed warning.

Increased risk of serious bacterial, fungal, viral, and opportunistic infections leading to hospitalization or death, including tuberculosis (TB). Interrupt treatment with RINVOQ if serious infection occurs until the infection is controlled. Test for latent TB before and during therapy; treat latent TB prior to use. Monitor all patients for active TB during treatment, even patients with initial negative, latent TB test.

Higher rate of all-cause mortality, including sudden cardiovascular death with another Janus kinase (JAK) inhibitor vs. tumor necrosis factor (TNF) blockers in rheumatoid arthritis (RA) patients.

Malignancies have occurred in patients treated with RINVOQ®. Higher rate of lymphomas and lung cancers with another JAK inhibitor vs. TNF blockers in RA patients.

Higher rate of MACE (defined as cardiovascular death, myocardial infarction, and stroke) with another JAK inhibitor vs. TNF blockers in RA patients.

Thrombosis has occurred in patients treated with RINVOQ®. Increased incidence of pulmonary embolism, venous and arterial thrombosis with another JAK inhibitor vs. TNF blockers.

### Indications and Usage

RINVOQ® is a Janus kinase (JAK) inhibitor indicated for the treatment of:
Adults with moderately to severely active rheumatoid arthritis who have had an inadequate response or intolerance to one or more TNF blockers.

### Limitations of Use

Use of RINVOQ® in combination with other JAK inhibitors, biologic DMARDs, or with potent immunosuppressants such as azathioprine and cyclosporine is not recommended.

Adults with active psoriatic arthritis who have had an inadequate response or intolerance to one or more TNF blockers.

### Limitations of Use

Use of RINVOQ® in combination with other JAK inhibitors, biologic DMARDs, or with potent immunosuppressants such as azathioprine and cyclosporine is not recommended.

Adults and pediatric patients 12 years of age and older with refractory, moderate to severe atopic dermatitis whose disease is not adequately controlled with other systemic drug products, including biologics, or when use of those therapies are inadvisable.

### Limitations of Use

RINVOQ® is not recommended for use in combination with other JAK inhibitors, biologic immunomodulators, or with other immunosuppressants.

### Dosage and Administration

Prior to treatment update immunizations and consider evaluating for active and latent tuberculosis, viral hepatitis, hepatic function, and pregnancy status. Avoid initiation or interrupt RINVOQ® if absolute lymphocyte count is less than 500 cells/mm³, absolute neutrophil count is less than 1000 cells/mm³, or hemoglobin level is less than 8 g/dL.

### Rheumatoid Arthritis

The recommended dosage of RINVOQ® is 15 mg once daily.

### Psoriatic Arthritis

The recommended dosage of RINVOQ® is 15 mg once daily.

### Atopic Dermatitis

Pediatric Patients 12 Years of Age and Older Weighing at Least 40 kg and Adults Less Than 65 Years of Age: Initiate treatment with 15 mg orally once daily. If an adequate response is not achieved, consider increasing the dosage to 30 mg orally once daily.
Adults 65 Years of Age and Older: Recommended dosage is 15 mg once daily.
Severe Renal Impairment: Recommended dosage is 15 mg once daily.

### Dosage Forms and Strengths

Extended-release tablets: 15 mg and 30 mg.

### Contraindications

Known hypersensitivity to upadacitinib or any of the excipients in RINVOQ®.

### Warnings and Precautions

Serious Infections: Avoid use of RINVOQ® in patients with active, serious infection, including localized infections.

Hypersensitivity: Serious hypersensitivity reactions (e.g., anaphylaxis) have been reported. Discontinue RINVOQ® if a serious hypersensitivity reaction occurs.

Gastrointestinal (GI) Perforations: Monitor patients at risk for GI perforations and promptly evaluate patients with symptoms.

Laboratory Abnormalities: Monitoring recommended due to potential changes in lymphocytes, neutrophils, hemoglobin, liver enzymes and lipids.

Embryo-Fetal Toxicity: RINVOQ® may cause fetal harm based on animal studies. Advise female patients of reproductive potential of the potential risk to a fetus and to use effective contraception.

Vaccinations: Avoid use of RINVOQ® with live vaccines.

### Adverse Reactions

Rheumatoid arthritis and psoriatic arthritis: Adverse reactions (≥1%) were: upper respiratory tract infections, herpes zoster, herpes simplex, bronchitis, nausea, cough, pyrexia and acne.

Atopic Dermatitis: Adverse reactions (≥1%) are: upper respiratory tract infections, acne, herpes simplex, headache, blood creatine phosphokinase increased, cough, hypersensitivity, folliculitis, nausea, abdominal pain, pyrexia, increased weight, herpes zoster, influenza, fatigue, neutropenia, myalgia, and influenza like illness.

### Drug Interactions

Strong CYP3A4 Inhibitors: Recommended dosage of RINVOQ® in patients taking strong CYP3A4 inhibitors is 15 mg once daily.

Strong CYP3A4 Inducers: Coadministration of RINVOQ® with strong CYP3A4 inducers is not recommended.

### Use in Specific Populations

Lactation: Advise not to breastfeed.

Hepatic Impairment: RINVOQ® is not recommended in patients with severe hepatic impairment.

### Full Prescribing Information

Warning: Serious Infections, Mortality, Malignancy, Major Adverse Cardiovascular Events, and Thrombosis

### Serious Infections

Patients treated with RINVOQ® are at increased risk for developing serious infections that may lead to hospitalization or death. Most patients who developed these infections were taking concomitant immunosuppressants such as methotrexate or corticosteroids. If a serious infection develops, interrupt RINVOQ® until the infection is controlled. Reported infections include:
Active tuberculosis, which may present with pulmonary or extrapulmonary disease. Patients should be tested for latent tuberculosis before RINVOQ® use and during therapy. Treatment for latent infection should be considered prior to RINVOQ® use.
Invasive fungal infections, including cryptococcosis and pneumocystosis.
Bacterial, viral, including herpes zoster, and other infections due to opportunistic pathogens.

The risks and benefits of treatment with RINVOQ® should be carefully considered prior to initiating therapy in patients with chronic or recurrent infection. Patients should be closely monitored for the development of signs and symptoms of infection during and after treatment with RINVOQ®, including the possible development of tuberculosis in patients who tested negative for latent tuberculosis infection prior to initiating therapy.

### Mortality

In a large, randomized, postmarketing safety study in rheumatoid arthritis (RA) patients 50 years of age and older with at least one cardiovascular risk factor comparing another Janus kinase (JAK) inhibitor to tumor necrosis factor (TNF) blockers, a higher rate of all-cause mortality, including sudden cardiovascular death, was observed with the JAK inhibitor.

### Malignancies

Lymphoma and other malignancies have been observed in patients treated with RINVOQ®. In RA patients treated with another JAK inhibitor, a higher rate of malignancies (excluding non-melanoma skin cancer (NMSC)) was observed when compared with TNF blockers. Patients who are current or past smokers are at additional increased risk.

### Major Adverse Cardiovascular Events

In RA patients 50 years of age and older with at least one cardiovascular risk factor treated with another JAK inhibitor, a higher rate of major adverse cardiovascular events (MACE) (defined as cardiovascular death, myocardial infarction, and stroke), was observed when compared with TNF blockers. Patients who are current or past smokers are at additional increased risk. Discontinue RINVOQ® in patients that have experienced a myocardial infarction or stroke.

### Thrombosis

Thrombosis, including deep venous thrombosis, pulmonary embolism, and arterial thrombosis have occurred in patients treated with JAK inhibitors used to treat inflammatory conditions. Many of these adverse events were serious and some resulted in death. In RA patients 50 years of age and older with at least one cardiovascular risk factor treated with another JAK inhibitor, a higher rate of thrombosis was observed when compared with TNF blockers. Avoid RINVOQ® in patients at risk. Patients with symptoms of thrombosis should discontinue RINVOQ® and be promptly evaluated.

### Indications and Usage

### Rheumatoid Arthritis

RINVOQ® (upadacitinib) is indicated for the treatment of adults with moderately to severely active rheumatoid arthritis who have had an inadequate response or intolerance to one or more TNF blockers.

Limitations of Use: Use of RINVOQ® in combination with other JAK inhibitors, biologic disease-modifying antirheumatic drugs (DMARDs), or with potent immunosuppressants such as azathioprine and cyclosporine, is not recommended.

### Psoriatic Arthritis

RINVOQ® is indicated for the treatment of adults with active psoriatic arthritis who have had an inadequate response or intolerance to one or more TNF blockers.

Limitations of Use: Use of RINVOQ® in combination with other JAK inhibitors, biologic DMARDs, or with potent immunosuppressants such as azathioprine and cyclosporine, is not recommended.

### Atopic Dermatitis

RINVOQ® is indicated for the treatment of adults and pediatric patients 12 years of age and older with refractory, moderate to severe atopic dermatitis whose disease is not adequately controlled with other systemic drug products, including biologics, or when use of those therapies are inadvisable.

Limitations of Use: RINVOQ® is not recommended for use in combination with other JAK inhibitors, biologic immunomodulators, or with other immunosuppressants.

### Dosage and Administration

### Recommended Evaluations and Immunizations Prior to Treatment Initiation

Prior to RINVOQ® treatment initiation, consider performing the following evaluations:
Active and latent tuberculosis (TB) infection evaluation—If positive, treat for TB prior to RINVOQ® use.
Viral hepatitis screening in accordance with clinical guidelines—RINVOQ® initiation is not recommended in patients with active hepatitis B or hepatitis C.
A complete blood count—RINVOQ® initiation is not recommended in patients with an absolute lymphocyte count less than 500 cells/mm3, absolute neutrophil count less than 1000 cells/mm3, or hemoglobin level less than 8 g/dL.
Baseline hepatic function: RINVOQ® initiation is not recommended for patients with severe hepatic impairment (Child-Pugh C).
Pregnancy Status: Verify the pregnancy status of females of reproductive potential prior to starting treatment].
Update immunizations according to current immunization guidelines.

### Important Administration Instructions

RINVOQ® tablets should be taken orally with or without food.

RINVOQ® tablets should be swallowed whole. RINVOQ® should not be split, crushed, or chewed.

### Recommended Dosage in Rheumatoid Arthritis

The recommended dosage of RINVOQ® is 15 mg once daily.

### Recommended Dosage in Psoriatic Arthritis

The recommended dosage of RINVOQ® is 15 mg once daily.

### Recommended Dosage in Atopic Dermatitis

Pediatric Patients 12 Years of Age and Older Weighing at Least 40 kg and Adults Less Than 65 Years of Age: Initiate treatment with 15 mg once daily. If an adequate response is not achieved, consider increasing the dosage to 30 mg once daily. Discontinue RINVOQ® if an adequate response is not achieved with the 30 mg dose. Use the lowest effective dose needed to maintain response.
Adults 65 Years of Age and Older: The recommended dosage is 15 mg once daily.
   Recommended Dosage in Patients with Renal Impairment or Severe Hepatic Impairment

### Renal Impairment

### Rheumatoid Arthritis and Psoriatic Arthritis:

No dosage adjustment is needed for patients with mild, moderate, or severe renal impairment.

### Atopic Dermatitis:

For patients with severe renal impairment [creatinine clearance (CrCL)<30 mL/min] the recommended dosage is 15 mg once daily. No dosage adjustment is needed for patients with mild or moderate renal impairment [(CrCL) >30 mL/min)].

### Hepatic Impairment

RINVOQ® is not recommended for use in patients with severe hepatic impairment

### Dosage Modifications Due to Drug Interactions

The recommended dosage in patients receiving strong CYP3A4 inhibitors is 15 mg once daily.

### Dosage Interruption

### Infections

If a patient develops a serious infection, including serious opportunistic infection, interrupt RINVOQ® treatment until the infection is controlled.

TABLE 1
Recommended Dosage Interruptions for Laboratory Abnormalities

| Laboratory Measure | Action |
|---|---|
| Absolute Neutrophil Count (ANC) | Interrupt treatment if ANC is less than 1000 cells/mm³; |
| | treatment may be restarted once ANC returns above this value |
| Absolute Lymphocyte Count (ALC) | Interrupt treatment if ALC is less than 500 cells/mm³; |
| | treatment may be restarted once ALC returns above this value |
| Hemoglobin (Hb) | Interrupt treatment if Hb is less than 8 g/dL; treatment |
| | may be restarted once Hb returns above this value |
| Hepatic transaminases | Interrupt treatment if drug-induced liver injury is |
| | suspected, until this diagnosis is excluded. |

Interruption of dosing may be needed for management of laboratory abnormalities as described in Table 1.

### Dosage Forms and Strengths

Extended-release tablets:
15 mg: purple, biconvex oblong, with dimensions of 14×8 mm, and debossed with ‘a15’ on one side.
30 mg: red, biconvex oblong, with dimensions of 14×8 mm, and debossed with ‘a30’ on one side.

### Contraindications

RINVOQ is contraindicated in patients with known hypersensitivity to upadacitinib or any of its excipients.

### Warnings and Precautions

### Serious Infections

Serious and sometimes fatal infections have been reported in patients receiving RINVOQ®. The most frequent serious infections reported with RINVOQ® included pneumonia and cellulitis. Among opportunistic infections, tuberculosis, multidermatomal herpes zoster, oral/esophageal candidiasis, and cryptococcosis, were reported with RINVOQ®. Avoid use of RINVOQ® in patients with an active, serious infection, including localized infections.

Consider the risks and benefits of treatment prior to initiating RINVOQ® in patients:
with chronic or recurrent infection
who have been exposed to tuberculosis
with a history of a serious or an opportunistic infection
who have resided or traveled in areas of endemic tuberculosis or endemic mycoses; or
with underlying conditions that may predispose them to infection.

Closely monitor patients for the development of signs and symptoms of infection during and after treatment with RINVOQ®. Interrupt RINVOQ® if a patient develops a serious or opportunistic infection. A patient who develops a new infection during treatment with RINVOQ® should undergo prompt and complete diagnostic testing appropriate for an immunocompromised patient; appropriate antimicrobial therapy should be initiated, the patient should be closely monitored, and RINVOQ® should be interrupted if the patient is not responding to antimicrobial therapy. RINVOQ® may be resumed once the infection is controlled.

### Tuberculosis

Evaluate and test patients for latent and active tuberculosis (TB) infection prior to administration of RINVOQ®. Patients with latent TB should be treated with standard antimycobacterial therapy before initiating RINVOQ®. RINVOQ® should not be given to patients with active TB. Consider anti-TB therapy prior to initiation of RINVOQ® in patients with previously untreated latent TB or active TB in whom an adequate course of treatment cannot be confirmed, and for patients with a negative test for latent TB but who have risk factors for TB infection. Consultation with a physician with expertise in the treatment of TB is recommended to aid in the decision about whether initiating anti-TB therapy is appropriate for an individual patient. During RINVOQ® use, monitor patients for the development of signs and symptoms of TB, including patients who tested negative for latent TB infection prior to initiating therapy.

### Viral Reactivation

Viral reactivation, including cases of herpes virus reactivation (e.g., herpes zoster) and hepatitis B virus reactivation, were reported in clinical trials with RINVOQ®. The risk of herpes zoster appears to be higher in patients treated with RINVOQ® in Japan. If a patient develops herpes zoster, consider temporarily interrupting RINVOQ® until the episode resolves. Screening for viral hepatitis and monitoring for reactivation should be performed in accordance with clinical guidelines before starting and during therapy with RINVOQ. Patients who were positive for hepatitis C antibody and hepatitis C virus RNA, were excluded from clinical trials. Patients who were positive for hepatitis B surface antigen or hepatitis B virus DNA were excluded from clinical trials. However, cases of hepatitis B reactivation were still reported in patients enrolled in the Phase 3 trials of RINVOQ. If hepatitis B virus DNA is detected while receiving RINVOQ®, a liver specialist should be consulted.

### Mortality

In a large, randomized, postmarketing safety study of another JAK inhibitor in RA patients 50 years of age and older with at least one cardiovascular risk factor, a higher rate of all-cause mortality, including sudden cardiovascular death, was observed in patients treated with the JAK inhibitor compared with TNF blockers. Consider the benefits and risks for the individual patient prior to initiating or continuing therapy with RINVOQ®.

### Malignancy and Lymphoproliferative Disorders

Malignancies, including lymphomas, were observed in clinical trials of RINVOQ®. In a large, randomized, postmarketing safety study of another JAK inhibitor in RA patients, a higher rate of malignancies (excluding non-melanoma skin cancer (NMSC)) was observed in patients treated with the JAK inhibitor compared to those treated with TNF blockers. A higher rate of lymphomas was observed in patients treated with the JAK inhibitor compared to those treated with TNF blockers. A higher rate of lung cancers was observed in current or past smokers treated with the JAK inhibitor compared to those treated with TNF blockers. In this study, current or past smokers had an additional increased risk of overall malignancies. Consider the benefits and risks for the individual patient prior to initiating or continuing therapy with RINVOQ®, particularly in patients with a known malignancy (other than a successfully treated NMSC), patients who develop a malignancy when on treatment, and patients who are current or past smokers.

### Non-Melanoma Skin Cancer

NMSCs have been reported in patients treated with RINVOQ®. Periodic skin examination is recommended for patients who are at increased risk for skin cancer. Exposure to sunlight and UV light should be limited by wearing protective clothing and using a broad-spectrum sunscreen.

### Major Adverse Cardiovascular Events

In a large, randomized, postmarketing safety study of another JAK inhibitor in RA patients 50 years of age and older with at least one cardiovascular risk factor, a higher rate of major adverse cardiovascular events (MACE) defined as cardiovascular death, non-fatal myocardial infarction (MI), and non-fatal stroke was observed with the JAK inhibitor compared to those treated with TNF blockers. Patients who are current or past smokers are at additional increased risk. Consider the benefits and risks for the individual patient prior to initiating or continuing therapy with RINVOQ®, particularly in patients who are current or past smokers and patients with other cardiovascular risk factors. Patients should be informed about the symptoms of serious cardiovascular events and the steps to take if they occur. Discontinue RINVOQ® in patients that have experienced a myocardial infarction or stroke.

### Thrombosis

Thrombosis, including deep venous thrombosis (DVT), pulmonary embolism (PE), and arterial thrombosis, have occurred in patients treated for inflammatory conditions with JAK inhibitors, including RINVOQ®. Many of these adverse events were serious and some resulted in death. In a large, randomized, postmarketing safety study of another JAK inhibitor in RA patients 50 years of age and older with at least one cardiovascular risk factor, higher rates of overall thrombosis, DVT, and PE were observed compared to those treated with TNF blockers. If symptoms of thrombosis occur, patients should discontinue RINVOQ® and be evaluated promptly and treated appropriately. Avoid RINVOQ® in patients that may be at increased risk of thrombosis.

### Hypersensitivity Reactions

Serious hypersensitivity reactions such as anaphylaxis and angioedema were reported in patients receiving RINVOQ® in clinical trials. If a clinically significant hypersensitivity reaction occurs, discontinue RINVOQ® and institute appropriate therapy.

### Gastrointestinal Perforations

Gastrointestinal perforations have been reported in clinical trials with RINVOQ®. In these trials, many patients with rheumatoid arthritis were receiving background therapy with nonsteroidal anti-inflammatory drugs (NSAIDs).

Monitor RINVOQ®-treated patients who may be at risk for gastrointestinal perforation (e.g., patients with a history of diverticulitis or taking NSAIDs). Evaluate promptly patients presenting with new onset abdominal pain for early identification of gastrointestinal perforation.

### Laboratory Abnormalities

### Neutropenia

Treatment with RINVOQ® was associated with an increased incidence of neutropenia (ANC less than 1000 cells/mm³). Evaluate neutrophil counts at baseline and thereafter according to routine patient management. Avoid RINVOQ® initiation and interrupt RINVOQ® treatment in patients with a low neutrophil count (i.e., ANC less than 1000 cells/mm³)

### Lymphopenia

ALC less than 500 cells/mm³ were reported in RINVOQ®-treated patients in clinical trials. Evaluate lymphocyte counts at baseline and thereafter according to routine patient management. Avoid RINVOQ® initiation or interrupt RINVOQ® treatment in patients with a low lymphocyte count (i.e., less than 500 cells/mm³).

### Anemia

Decreases in hemoglobin levels to less than 8 g/dL were reported in RINVOQ®Q-treated patients in clinical trials. Evaluate hemoglobin at baseline and thereafter according to routine patient management. Avoid RINVOQ® initiation or interrupt RINVOQ® treatment in patients with a low hemoglobin level (i.e., less than 8 g/dL).

### Lipids

Treatment with RINVOQ® was associated with increases in lipid parameters, including total cholesterol, low-density lipoprotein (LDL) cholesterol, and high-density lipoprotein (HDL) cholesterol. Elevations in LDL cholesterol decreased to pre-treatment levels in response to statin therapy. The effect of these lipid parameter elevations on cardiovascular morbidity and mortality has not been determined. Assess lipid parameters approximately 12 weeks after initiation of treatment, and thereafter according to the clinical guidelines for hyperlipidemia. Manage patients according to clinical guidelines for the management of hyperlipidemia.

### Liver Enzyme Elevations

Treatment with RINVOQ® was associated with increased incidence of liver enzyme elevations compared to treatment with placebo. Evaluate liver enzymes at baseline and thereafter according to routine patient management. Prompt investigation of the cause of liver enzyme elevation is recommended to identify potential cases of drug-induced liver injury. If increases in ALT or AST are observed during routine patient management and drug-induced liver injury is suspected, RINVOQ® should be interrupted until this diagnosis is excluded.

### Embryo-Fetal Toxicity

Based on findings in animal studies, RINVOQ® may cause fetal harm when administered to a pregnant woman. Administration of upadacitinib to rats and rabbits during organogenesis caused increases in fetal malformations. Verify the pregnancy status of patients of reproductive potential prior to starting treatment. Advise females of reproductive potential of the potential risk to the fetus and to use effective contraception during treatment with RINVOQ® and for 4 weeks following completion of therapy.

### Vaccinations

Avoid use of live vaccines during, or immediately prior to, RINVOQ® therapy. Prior to initiating RINVOQ®, it is recommended that patients be brought up to date with all immunizations, including varicella zoster or prophylactic herpes zoster vaccinations, in agreement with current immunization guidelines.

### Adverse Reactions

The following clinically significant adverse reactions are described elsewhere in the labeling:
Serious Infections
Mortality
Malignancy and Lymphoproliferative Disorders
Major Adverse Cardiovascular Events
Thrombosis
Hypersensitivity Reactions
Gastrointestinal Perforations
Laboratory Abnormalities

### Clinical Trials Experience

Because clinical trials are conducted under widely varying conditions, adverse reaction rates observed in the clinical trials of a drug cannot be directly compared to rates in the clinical trials of another drug and may not reflect the rates observed in practice.

Adverse Reactions in Patients with Rheumatoid Arthritis

A total of 3833 patients with rheumatoid arthritis were treated with upadacitinib in the Phase 3 clinical trials of whom 2806 were exposed for at least one year. Patients could advance or switch to RINVOQ® 15 mg from placebo, or be rescued to RINVOQ® from active comparator or placebo from as early as Week 12 depending on the trial design. A total of 2630 patients received at least 1 dose of RINVOQ® 15 mg, of whom 1860 were exposed for at least one year. In trials RA-I, RA-II, RA-III and RA-V, 1213 patients received at least 1 dose of RINVOQ®15 mg, of which 986 patients were exposed for at least one year, and 1203 patients received at least 1 dose of upadacitinib 30 mg, of which 946 were exposed for at least one year.

TABLE 2
Adverse Reactions Reported in ≥1% of Rheumatoid Arthritis
Patients Treated with RINVOQ 15 mg in Placebo-controlled Trials

| | Placebo | RINVOQ 15 mg |
|---|---|---|
| | n = 1042 | n = 1035 |
| Adverse Reaction | (%) | (%) |
| Upper respiratory tract | 9.5 | 13.5 |
| infection (URTI)* | | |
| Nausea | 2.2 | 3.5 |
| Cough | 1.0 | 2.2 |
| Pyrexia | 0 | 1.2 |

*URTI includes: acute sinusitis, laryngitis, nasopharyngitis, oropharyngeal pain, pharyngitis, pharyngotonsillitis, rhinitis, sinusitis, tonsillitis, viral upper respiratory tract infection

Other adverse reactions reported in less than 1% of patients in the RINVOQ® 15 mg group and at a higher rate than in the placebo group through Week 12 included pneumonia, herpes zoster, herpes simplex (includes oral herpes), and oral candidiasis. Four integrated datasets are presented in the Specific Adverse Reaction section:

Placebo-controlled Trials: Trials RA-III, RA-IV, and RA-V were integrated to represent safety through 12/14 weeks for placebo (n=1042) and RINVOQ® 15 mg (n=1035). Trials RA-III and RA-V were integrated to represent safety through 12 weeks for placebo (n=390), RINVOQ® 15 mg (n=385), and upadacitinib 30 mg (n=384). Trial RA-IV did not include the 30 mg dose and, therefore, safety data for upadacitinib 30 mg can only be compared with placebo and RINVOQ 15 mg rates from pooling trials RA-III and RA-V. MTX-controlled Trials: Trials RA-I and RA-II were integrated to represent safety through 12/14 weeks for MTX (n=530), RINVOQ 15 mg (n=534), and upadacitinib 30 mg (n=529). 12-Month Exposure Dataset: Trials RA-I, II, III, and V were integrated to represent the long-term safety of RINVOQ® 15 mg (n=1213) and upadacitinib 30 mg (n=1203). Exposure adjusted incidence rates were adjusted by trial for all the adverse events reported in this section.

### Specific Adverse Reactions

### Infections

Placebo-controlled Trials: In RA-III, RA-IV, and RA-V, infections were reported in 218 patients (95.7 per 100 patient-years) treated with placebo and 284 patients (127.8 per 100 patient-years) treated with RINVOQ® 15 mg. In RA-III and RA-V, infections were reported in 99 patients (136.5 per 100 patient-years) treated with placebo, 118 patients (164.5 per 100 patient-years) treated with RINVOQ® 15 mg, and 126 patients (180.3 per 100 patient-years) treated with upadacitinib 30 mg.

MTX-controlled Trials: Infections were reported in 127 patients (119.5 per 100 patient-years) treated with MTX monotherapy, 104 patients (91.8 per 100 patient-years) treated with RINVOQ 15 mg monotherapy, and 128 patients (115.1 per 100 patient-years) treated with upadacitinib 30 mg monotherapy.

12-Month Exposure Dataset: Infections were reported in 615 patients (83.8 per 100 patient-years) treated with RINVOQ® 15 mg and 674 patients (99.7 per 100 patient-years) treated with upadacitinib 30 mg.

### Serious Infections

Placebo-controlled Trials: In RA-III, RA-IV, and RA-V, serious infections were reported in 6 patients (2.3 per 100 patient-years) treated with placebo, and 12 patients (4.6 per 100 patient-years) treated with RINVOQ® 15 mg. In RA-III and RA-V, serious infections were reported in 1 patient (1.2 per 100 patient-years) treated with placebo, 2 patients (2.3 per 100 patient-years) treated with RINVOQ® 15 mg, and 7 patients (8.2 per 100 patient-years) treated with upadacitinib 30 mg.

MTX-controlled Trials: Serious infections were reported in 2 patients (1.6 per 100 patient-years) treated with MTX monotherapy, 3 patients (2.4 per 100 patient-years) treated with RINVOQ® 15 mg monotherapy, and 8 patients (6.4 per 100 patient-years) treated with upadacitinib 30 mg monotherapy.

12-Month Exposure Dataset: Serious infections were reported in 38 patients (3.5 per 100 patient-years) treated with RINVOQ® 15 mg and 59 patients (5.6 per 100 patient-years) treated with upadacitinib 30 mg.

The most frequently reported serious infections were pneumonia and cellulitis.

### Tuberculosis

Placebo-controlled Trials and MTX-controlled Trials: In the placebo-controlled period, there were no active cases of tuberculosis reported in the placebo, RINVOQ® 15 mg, and upadacitinib 30 mg groups. In the MTX-controlled period, there were no active cases of tuberculosis reported in the MTX monotherapy, RINVOQ® 15 mg monotherapy, and upadacitinib 30 mg monotherapy groups.

12-Month Exposure Dataset: Active tuberculosis was reported for 2 patients treated with RINVOQ® 15 mg and 1 patient treated with upadacitinib 30 mg. Cases of extra-pulmonary tuberculosis were reported.

### Opportunistic Infections (Excluding Tuberculosis)

Placebo-controlled Trials: In RA-III, RA-IV, and RA-V, opportunistic infections were reported in 3 patients (1.2 per 100 patient-years) treated with placebo, and 5 patients (1.9 per 100 patient-years) treated with RINVOQ® 15 mg. In RA-III and RA-V, opportunistic infections were reported in 1 patient (1.2 per 100 patient-years) treated with placebo, 2 patients (2.3 per 100 patient-years) treated with RINVOQ® 15 mg, and 6 patients (7.1 per 100 patient-years) treated with upadacitinib 30 mg.

MTX-controlled Trials: Opportunistic infections were reported in 1 patient (0.8 per 100 patient-years) treated with MTX monotherapy, 0 patients treated with RINVOQ® 15 mg monotherapy, and 4 patients (3.2 per 100 patient-years) treated with upadacitinib 30 mg monotherapy.

12-Month Exposure Dataset: Opportunistic infections were reported in 7 patients (0.6 per 100 patient-years) treated with RINVOQ® 15 mg and 15 patients (1.4 per 100 patient-years) treated with upadacitinib 30 mg.

### Malignancies

Placebo-controlled Trials: In RA-III, RA-IV, and RA-V, malignancies excluding NMSC were reported in 1 patient (0.4 per 100 patient-years) treated with placebo, and 1 patient (0.4 per 100 patient-years) treated with RINVOQ® 15 mg. In RA-III and RA-V, malignancies excluding NMSC were reported in 0 patients treated with placebo, 1 patient (1.1 per 100 patient-years) treated with RINVOQ 15 mg, and 3 patients (3.5 per 100 patient-years) treated with upadacitinib 30 mg.

MTX-controlled Trials: Malignancies excluding NMSC were reported in 1 patient (0.8 per 100 patient-years) treated with MTX monotherapy, 3 patients (2.4 per 100 patient-years) treated with RINVOQ® 15 mg monotherapy, and 0 patients treated with upadacitinib 30 mg monotherapy.

12-Month Exposure Dataset: Malignancies excluding NMSC were reported in 13 patients (1.2 per 100 patient-years) treated with RINVOQ® 15 mg and 14 patients (1.3 per 100 patient-years) treated with upadacitinib 30 mg.

### Gastrointestinal Perforations

Placebo-controlled Trials: There were no gastrointestinal perforations (based on medical review) reported in patients treated with placebo, RINVOQ® 15 mg, and upadacitinib 30 mg.

MTX-controlled Trials: There were no cases of gastrointestinal perforations reported in the MTX and RINVOQ® 15 mg group through 12/14 weeks. Two cases of gastrointestinal perforations were observed in the upadacitinib 30 mg group.

12-Month Exposure Dataset: Gastrointestinal perforations were reported in 1 patient treated with RINVOQ 15 mg and 4 patients treated with upadacitinib 30 mg.

### Thrombosis

Placebo-controlled Trials: In RA-IV, venous thrombosis (pulmonary embolism or deep vein thrombosis) was observed in 1 patient treated with placebo and 1 patient treated with RINVOQ® 15 mg. In RA-V, venous thrombosis was observed in 1 patient treated with RINVOQ® 15 mg. There were no observed cases of venous thrombosis reported in RA-III. No cases of arterial thrombosis were observed through 12/14 weeks.

MTX-controlled Trials: In RA-II, venous thrombosis was observed in 0 patients treated with MTX monotherapy, 1 patient treated with RINVOQ® 15 mg monotherapy and 0 patients treated with upadacitinib 30 mg monotherapy through Week 14. In RA-II, no cases of arterial thrombosis were observed through 12/14 weeks. In RA-I, venous thrombosis was observed in 1 patient treated with MTX, 0 patients treated with RINVOQ® 15 mg and 1 patient treated with upadacitinib 30 mg through Week 24. In RA-I, arterial thrombosis was observed in 1 patient treated with upadacitinib 30 mg through Week 24.

12-Month Exposure Dataset: Venous thrombosis events were reported in 5 patients (0.5 per 100 patient-years) treated with RINVOQ® 15 mg and 4 patients (0.4 per 100 patient-years) treated with upadacitinib 30 mg. Arterial thrombosis events were reported in 0 patients treated with RINVOQ® 15 mg and 2 patients (0.2 per 100 patient-years) treated with upadacitinib 30 mg.

### Laboratory Abnormalities

### Hepatic Transaminase Elevations

In placebo-controlled trials (RA-III, RA-IV, and RA-V) with background DMARDs, for up to 12/14 weeks, alanine transaminase (ALT) and aspartate transaminase (AST) elevations ≥3×upper limit of normal (ULN) in at least one measurement were observed in 2.1% and 1.5% of patients treated with RINVOQ® 15 mg, and in 1.5% and 0.7% of patients treated with placebo, respectively. In RA-III and RA-V, ALT and AST elevations ≥3×ULN in at least one measurement were observed in 0.8% and 1.0% of patients treated with RINVOQ® 15 mg, 1.0% and 0% of patients treated with upadacitinib 30 mg and in 1.3% and 1.0% of patients treated with placebo, respectively.

In MTX-controlled trials, for up to 12/14 weeks, ALT and AST elevations ≥3×ULN in at least one measurement were observed in 0.8% and 0.4% of patients treated with RINVOQ®15 mg, 1.7% and 1.3% of patients treated with upadacitinib 30 mg and in 1.9% and 0.9% of patients treated with MTX, respectively.

### Lipid Elevations

Upadacitinib treatment was associated with dose-related increases in total cholesterol, triglycerides and LDL cholesterol. Upadacitinib was also associated with increases in HDL cholesterol. Elevations in LDL and HDL cholesterol peaked by Week 8 and remained stable thereafter. In controlled trials, for up to 12/14 weeks, changes from baseline in lipid parameters in patients treated with RINVOQ® 15 mg and upadacitinib 30 mg, respectively, are summarized below:
Mean LDL cholesterol increased by 14.81 mg/dL and 17.17 mg/dL.
Mean HDL cholesterol increased by 8.16 mg/dL and 9.01 mg/dL.
The mean LDL/HDL ratio remained stable.
Mean triglycerides increased by 13.55 mg/dL and 14.44 mg/dL.

In placebo-controlled trials (RA-III, RA-IV, and RA-V) with background DMARDs, for up to 12/14 weeks, dose-related increases in creatine phosphokinase (CPK) values were observed. CPK elevations >5×ULN were reported in 1.0%, and 0.3% of patients over 12/14 weeks in the RINVOQ® 15 mg and placebo groups, respectively. Most elevations >5×ULN were transient and did not require treatment discontinuation. In RA-III and RA-V, CPK elevations >5×ULN were observed in 0.3% of patients treated with placebo, 1.6% of patients treated with RINVOQ® 15 mg, and none in patients treated with upadacitinib 30 mg.

### Neutropenia

In placebo-controlled trials (RA-III, RA-IV, and RA-V) with background DMARDs, for up to 12/14 weeks, dose-related decreases in neutrophil counts, below 1000 cells/mm³ in at least one measurement occurred in 1.1% and <0.1% of patients in the RINVOQ® 15 mg and placebo groups, respectively. In RA-III and RA-V, decreases in neutrophil counts below 1000 cells/mm³ in at least one measurement occurred in 0.3% of patients treated with placebo, 1.3% of patients treated with RINVOQ® 15 mg, and 2.4% of patients treated with upadacitinib 30 mg. In clinical trials, treatment was interrupted in response to ANC less than 1000 cells/mm³.

### Lymphopenia

In placebo-controlled trials (RA-III, RA-IV, and RA-V) with background DMARDs, for up to 12/14 weeks, dose-related decreases in lymphocyte counts below 500 cells/mm³ in at least one measurement occurred in 0.9% and 0.7% of patients in the RINVOQ 15 mg and placebo groups, respectively. In RA-III and RA-V, decreases in lymphocyte counts below 500 cells/mm³ in at least one measurement occurred in 0.5% of patients treated with placebo, 0.5% of patients treated with RINVOQ® 15 mg, and 2.4% of patients treated with upadacitinib 30 mg.

### Anemia

In placebo-controlled trials (RA-III, RA-IV, and RA-V) with background DMARDs, for up to 12/14 weeks, hemoglobin decreases below 8 g/dL in at least one measurement occurred in <0.1% of patients in both the RINVOQ® 15 mg and placebo groups. In RA-III and RA-V, hemoglobin decreases below 8 g/dL in at least one measurement were observed in 0.3% of patients treated with placebo, and none in patients treated with RINVOQ® 15 mg and upadacitinib 30 mg.

Adverse Reactions in Patients with Psoriatic Arthritis

A total of 1827 patients with psoriatic arthritis were treated with upadacitinib in clinical studies representing 1639.2 patient-years of exposure, of whom 722 were exposed to upadacitinib for at least one year. In the two Phase 3 studies, 907 patients received at least 1 dose of RINVOQ® 15 mg, of whom 359 were exposed for at least one year. Two placebo-controlled studies were integrated (640 patients on RINVOQ® 15 mg once daily and 635 patients on placebo) to evaluate the safety of RINVOQ® 15 mg in comparison to placebo for up to 24 weeks after treatment initiation. Overall, the safety profile observed in patients with active psoriatic arthritis treated with RINVOQ® 15 mg was consistent with the safety profile observed in patients with rheumatoid arthritis. During the 24-week placebo-controlled period, the frequencies of herpes zoster and herpes simplex were >1% (1.1% and 1.4%, respectively) with RINVOQ® 15 mg and 0.8% and 1.3%, respectively with placebo. A higher incidence of acne and bronchitis was also observed in patients treated with RINVOQ® 15 mg (1.3% and 3.9%, respectively) compared to placebo (0.3% and 2.7%, respectively).

Adverse Reactions in Patients with Atopic Dermatitis

Three Phase 3 (AD-1, AD-2, and AD-3) and one Phase 2b (AD-4) randomized, double-blind, placebo-controlled, multicenter trials evaluated the safety of RINVOQ® in patients with moderate-to-severe atopic dermatitis. The majority of patients were White (68%) and male (57%). The mean age was 34 years (ranged from 12 to 75 years) and 13% of the patients were 12 to less than 18 years. In these 4 trials, 2612 patients were treated with RINVOQ® 15 mg or 30 mg orally once daily, with or without concomitant topical corticosteroids (TCS). In the Phase 3 clinical trials (AD-1, AD-2, and AD-3), a total of 1239 patients received RINVOQ®15 mg, of whom 791 were exposed for at least one year and 1246 patients received RINVOQ® 30 mg, of whom 826 were exposed for at least one year. Trials AD-1, AD-2, and AD-4 compared the safety of RINVOQ® monotherapy to placebo through Week 16. Trial AD-3 compared the safety of RINVOQ®+TCS to placebo+TCS through Week 16.

### Weeks 0 to 16 (Trials AD-1 to AD-4)

In RINVOQ® trials with and without TCS (Trials AD-1, 2, 3 and 4) through Week 16, the proportion of patients who discontinued treatment because of adverse reactions in the RINVOQ® 15 mg, 30 mg and placebo groups were 2.3%, 2.9% and 3.8%, respectively. Table 3 summarizes the adverse reactions that occurred at a rate of at least 1% in the RINVOQ® 15 mg or 30 mg groups during the first 16 weeks of treatment.

TABLE 3
Adverse Reactions Reported in ≥1% of Patients with Atopic Dermatitis
Treated with RINVOQ ® 15 mg or 30 mg

| | | RINVOQ | RINVOQ |
|---|---|---|---|
| | Placebo | 15 mg | 30 mg |
| | n = 902 | n = 899 | n = 906 |
| Adverse Reaction | (%) | (%) | (%) |
| Upper respiratory tract | 17 | 23 | 25 |
| infection (URTI)* | | | |
| Acne** | 2 | 10 | 16 |
| Herpes simplex*** | 2 | 4 | 8 |
| Headache | 4 | 6 | 6 |
| Increased blood creatine | 2 | 5 | 6 |
| phosphokinase | | | |
| Cough | 1 | 3 | 3 |
| Hypersensitivity**** | 2 | 2 | 3 |
| Folliculitis | 1 | 2 | 3 |
| Nausea | 1 | 3 | 3 |
| Abdominal pain***** | 1 | 3 | 2 |
| Pyrexia | 1 | 2 | 2 |
| Increased Weight | 1 | 2 | 2 |
| Herpes zoster****** | 1 | 2 | 2 |
| Influenza | <1 | 2 | 2 |
| Fatigue | 1 | 1 | 2 |
| Neutropenia | <1 | 1 | 2 |
| Myalgia | 1 | 1 | 2 |
| Influenza like illness | 1 | 1 | 2 |

*Includes: laryngitis, laryngitis viral, nasopharyngitis, oropharyngeal pain, pharyngeal abscess, pharyngitis, pharyngitis streptococcal, pharyngotonsillitis, respiratory tract infection, respiratory tract infection viral, rhinitis, rhinolaryngitis, sinusitis, tonsillitis, tonsillitis bacterial, upper respiratory tract infection, viral pharyngitis, viral upper respiratory tract infection
**Includes: acne and dermatitis acneiform
***Includes: genital herpes, genital herpes simplex, herpes dermatitis, herpes ophthalmic, herpes simplex, nasal herpes, ophthalmic herpes simplex, herpes virus infection, oral herpes
****Includes anaphylactic reaction, anaphylactic shock, angioedema, dermatitis exfoliative generalized, drug hypersensitivity, eyelid oedema, face oedema, hypersensitivity, periorbital swelling, pharyngeal swelling, swelling face, toxic skin eruption, type I hypersensitivity, urticaria
*****Includes abdominal pain and abdominal pain upper
******Includes herpes zoster and varicella

Other adverse reactions reported in less than 1% of patients in the RINVOQ®15 mg and/or 30 mg group and at a higher rate than in the placebo group through Week 16 included anemia, oral candidiasis, pneumonia, and the adverse event of retinal detachment. The safety profile of RINVOQ® through Week 52 was generally consistent with the safety profile observed at Week 16. Overall, the safety profile observed in patients with AD treated with RINVOQ® was similar to the safety profile in patients with RA. Other specific adverse reactions that were reported in patients with AD included eczema herpeticum/Kaposi's varicelliform eruption.

### Eczema Herpeticum/Kaposi's Varicelliform Eruption

Placebo-controlled Period (16 weeks): Eczema herpeticum was reported in 4 patients (1.6 per 100 patient-years) treated with placebo, 6 patients (2.2 per 100 patient-years) treated with RINVOQ® 15 mg and 7 patients (2.6 per 100 patient-years) treated with RINVOQ® 30 mg.

12-Month Exposure (Weeks 0 to 52): Eczema herpeticum was reported in 18 patients (1.6 per 100 patient-years) treated with RINVOQ® 15 mg and 17 patients (1.5 per 100 patient-years) treated with RINVOQ® 30 mg.

### Drug Interactions

### Strong CYP3A4 Inhibitors

Upadacitinib exposure is increased when RINVOQ® is co-administered with a strong CYP3A4 inhibitor (such as ketoconazole), which may increase the risk of RINVOQ® adverse reactions. Monitor patients closely for adverse reactions when co-administering RINVOQ 15 mg once daily with strong CYP3A4 inhibitors. Coadministration of RINVOQ® 30 mg once daily with strong CYP3A4 inhibitors is not recommended.

### Strong CYP3A4 Inducers

Upadacitinib exposure is decreased when RINVOQ® is co-administered with strong CYP3A4 inducers (such as rifampin), which may lead to reduced therapeutic effect of RINVOQ®. Coadministration of RINVOQ® with strong CYP3A4 inducers is not recommended.

### Use in Specific Populations

### Pregnancy

### Risk Summary

Available data from the pharmacovigilance safety database and postmarketing case reports on use of RINVOQ® in pregnant women are not sufficient to evaluate a drug-associated risk for major birth defects or miscarriage. Based on animal studies, RINVOQ® has the potential to adversely affect a developing fetus. Advise patients of reproductive potential and pregnant patients of the potential risk to the fetus. In animal embryo-fetal development studies, oral upadacitinib administration to pregnant rats and rabbits at exposures equal to or greater than approximately 2 and 17 times the 15 mg dose and 0.9 and 8.5 times the maximum recommended human dose (MRHD) of 30 mg, respectively, resulted in dose-related increases in skeletal malformations (rats only), an increased incidence of cardiovascular malformations (rabbits only), increased post-implantation loss (rabbits only), and decreased fetal body weights in both rats and rabbits. No developmental toxicity was observed in pregnant rats and rabbits treated with oral upadacitinib during organogenesis at approximately 0.3 and 2.5 times the 15 mg dose and 0.2 and 1.3 times the MRHD of 30 mg, respectively. In a pre- and post-natal development study in pregnant female rats, oral upadacitinib administration at exposures approximately 1.6 times the MRHD of 30 mg resulted in no maternal or developmental toxicity.

The background risks of major birth defects and miscarriage for the indicated populations are unknown. All pregnancies have a background risk of birth defect, loss, or other adverse outcomes. In the U.S. general population, the estimated background risk of major birth defects and miscarriages are 2-4% and 15-20%, respectively.

### Clinical Considerations

Disease-Associated Maternal and/or Embryo/Fetal Risk

Published data suggest that increased disease activity is associated with the risk of developing adverse pregnancy outcomes in women with rheumatoid arthritis. Adverse pregnancy outcomes include preterm delivery (before 37 weeks of gestation), low birth weight (less than 2500 g) infants, and small for gestational age at birth.

### Data

### Animal Data

In an oral embryo-fetal development study, pregnant rats received upadacitinib at doses of 5, 25, and 75 mg/kg/day during the period of organogenesis from gestation day 6 to 17. Upadacitinib was teratogenic (skeletal malformations that consisted of misshapen humerus and bent scapula) at exposures equal to or greater than approximately 1.0 times the MRHD of 30 mg (on an AUC basis at maternal oral doses of 5 mg/kg/day and higher). Additional skeletal malformations (bent forelimbs/hindlimbs and rib/vertebral defects) and decreased fetal body weights were observed in the absence of maternal toxicity at an exposure approximately 48 times the MRHD of 30 mg (on an AUC basis at a maternal oral dose of 75 mg/kg/day).

In a second oral embryo-fetal development study, pregnant rats received upadacitinib at doses of 1.5 and 4 mg/kg/day during the period of organogenesis from gestation day 6 to 17. Upadacitinib was teratogenic (skeletal malformations that included bent humerus and scapula) at exposures approximately 0.9 times the MRHD of 30 mg (on an AUC basis at maternal oral doses of 4 mg/kg/day). No developmental toxicity was observed in rats at an exposure approximately 0.2 times the MRHD of 30 mg (on an AUC basis at a maternal oral dose of 1.5 mg/kg/day).

In an oral embryo-fetal developmental study, pregnant rabbits received upadacitinib at doses of 2.5, 10, and 25 mg/kg/day during the period of organogenesis from gestation day 7 to 19. Embryolethality, decreased fetal body weights, and cardiovascular malformations were observed in the presence of maternal toxicity at an exposure approximately 8.5 times the MRHD of 30 mg (on an AUC basis at a maternal oral dose of 25 mg/kg/day). Embryolethality consisted of increased post-implantation loss that was due to elevated incidences of both total and early resorptions. No developmental toxicity was observed in rabbits at an exposure approximately 1.3 times the MRHD of 30 mg (on an AUC basis at a maternal oral dose of 10 mg/kg/day).

In an oral pre- and post-natal development study, pregnant female rats received upadacitinib at doses of 2.5, 5, and 10 mg/kg/day from gestation day 6 through lactation day 20. No maternal or developmental toxicity was observed in either mothers or offspring, respectively, at an exposure approximately 1.6 times the MRHD of 30 mg (on an AUC basis at a maternal oral dose of 10 mg/kg/day).

### Lactation

### Risk Summary

There are no data on the presence of upadacitinib in human milk, the effects on the breastfed infant, or the effects on milk production. Available pharmacodynamic/toxicological data in animals have shown excretion of upadacitinib in milk (see Data). When a drug is present in animal milk, it is likely that the drug will be present in human milk. Because of the potential for serious adverse reactions in the breastfed infant, advise patients that breastfeeding is not recommended during treatment with RINVOQ®, and for 6 days (approximately 10 half-lives) after the last dose.

### Data

A single oral dose of 10 mg/kg radiolabeled upadacitinib was administered to lactating female Sprague-Dawley rats on post-partum days 7-8. Drug exposure was approximately 30-fold greater in milk than in maternal plasma based on AUC₀₋ₜ values. Approximately 97% of drug-related material in milk was parent drug.

### Females and Males of Reproductive Potential

### Pregnancy Testing

Verify the pregnancy status of females of reproductive potential prior to starting treatment with RINVOQ®.

### Contraception

### Females

Based on animal studies, upadacitinib may cause embryo-fetal harm when administered to pregnant women. Advise female patients of reproductive potential to use effective contraception during treatment with RINVOQ® and for 4 weeks after the final dose.

### Pediatric Use

### Juvenile Idiopathic Arthritis and Psoriatic Arthritis

The safety and effectiveness of RINVOQ® in pediatric patients with juvenile idiopathic arthritis and psoriatic arthritis have not been established.

### Atopic Dermatitis

The safety and effectiveness of RINVOQ® in pediatric patients 12 years of age and older weighing at least 40 kg with atopic dermatitis have been established. A total of 344 pediatric patients aged 12 to 17 years with moderate to severe atopic dermatitis were randomized across three trials (AD-1, AD-2 and AD-3) to receive either RINVOQ® 15 mg (N=114) or 30 mg (N=114) or matching placebo (N=116) in monotherapy or combination with topical corticosteroids. Efficacy was consistent between the pediatric patients and adults. The adverse reaction profile in the pediatric patients was similar to the adults. The safety and effectiveness of RINVOQ® in pediatric patients less than 12 years of age with atopic dermatitis have not been established.

### Geriatric Use

### Rheumatoid Arthritis and Psoriatic Arthritis

Of the 4381 patients treated in the five clinical studies, a total of 906 rheumatoid arthritis patients were 65 years of age or older, including 146 patients 75 years and older. Of the 1827 patients treated in the two psoriatic arthritis Phase 3 clinical studies, a total of 274 patients were 65 years of age or older, including 34 patients 75 years and older. No differences in effectiveness were observed between these patients and younger patients; however, there was a higher rate of overall adverse events, including serious infections, in patients 65 years of age and older.

### Atopic Dermatitis

Of the 2583 patients treated in the three Phase 3 clinical trials, a total of 120 patients with atopic dermatitis were 65 years of age or older, including 6 patients 75 years of age. No differences in effectiveness were observed between these patients and younger patients; however, there was a higher rate of serious infections and malignancies in those patients 65 years of age or older in the 30 mg dosing group in the long-term trials.

### Renal Impairment

For patients with rheumatoid arthritis and psoriatic arthritis, no dosage adjustment is needed in patients with mild, moderate or severe renal impairment.

For patients with atopic dermatitis, the maximum recommended dosage is 15 mg once daily for patients with severe renal impairment (CrCL <30 mL/min). No dosage adjustment is needed in patients with mild or moderate renal impairment.

The use of RINVOQ® has not been studied in patients with end stage renal disease, and therefore not recommended for use in this population.

### Hepatic Impairment

No dosage adjustment is needed in patients with mild (Child Pugh A) or moderate (Child Pugh B) hepatic impairment. The use of RINVOQ® has not been studied in patients with severe hepatic impairment (Child Pugh C), and therefore not recommended for use in this population.

### Description

RINVOQ® is formulated with upadacitinib, a JAK inhibitor. Upadacitinib has the following chemical name: (3S,4R)-3-Ethyl-4-(3H-imidazo[1,2-a]pyrrolo[2,3-e]pyrazin-8-yl)-N-(2,2,2-trifluoroethyl)pyrrolidine-1-carboxamide hydrate (2:1). The strength of upadacitinib is based on anhydrous upadacitinib. The solubility of upadacitinib in water is 38 to less than 0.2 mg/mL across a pH range of 2 to 9 at 37° C. Upadacitinib has a molecular weight of 389.38 g/mol and a molecular formula of C₁₇H₁₉F₃N₆O·½H2O. The chemical structure of upadacitinib is:

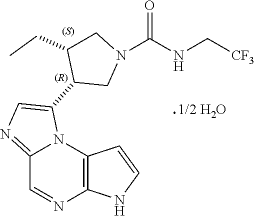

RINVOQ® 15 mg extended-release tablets for oral administration are purple, biconvex oblong, with dimensions of 14×8 mm, and debossed with ‘a15’ on one side. Each tablet contains the following inactive ingredients: colloidal silicon dioxide, ferrosoferric oxide, hypromellose, iron oxide red, magnesium stearate, mannitol, microcrystalline cellulose, polyvinyl alcohol, polyethylene glycol, talc, tartaric acid and titanium dioxide.

RINVOQ® 30 mg extended-release tablets for oral administration are red, biconvex oblong, with dimensions of 14×8 mm, and debossed with ‘a30’ on one side. Each tablet contains the following inactive ingredients: colloidal silicon dioxide, hypromellose, iron oxide red, magnesium stearate, mannitol, microcrystalline cellulose, polyvinyl alcohol, polyethylene glycol, talc, tartaric acid and titanium dioxide.

### Clinical Pharmacology

### Mechanism of Action

Upadacitinib is a Janus kinase (JAK) inhibitor. JAKs are intracellular enzymes which transmit signals arising from cytokine or growth factor-receptor interactions on the cellular membrane to influence cellular processes of hematopoiesis and immune cell function. Within the signaling pathway, JAKs phosphorylate and activate signal transducers and activators of transcription (STATs) which modulate intracellular activity including gene expression. Upadacitinib modulates the signaling pathway at the point of JAKs, preventing the phosphorylation and activation of STATs.

JAK enzymes transmit cytokine signaling through their pairing (e.g., JAK1/JAK2, JAK1/JAK3, JAK1/TYK2, JAK2/JAK2, JAK2/TYK2). In a cell-free isolated enzyme assay, upadacitinib had greater inhibitory potency at JAK1 and JAK2 relative to JAK3 and TYK2. In human leukocyte cellular assays, upadacitinib inhibited cytokine-induced STAT phosphorylation mediated by JAK1 and JAK1/JAK3 more potently than JAK2/JAK2 mediated STAT phosphorylation. However, the relevance of inhibition of specific JAK enzymes to therapeutic effectiveness is not currently known.

### Pharmacodynamics

### Inhibition of IL-6 Induced STAT3 and IL-7 Induced STAT5 Phosphorylation

In healthy volunteers, the administration of upadacitinib (immediate release formulation) resulted in a dose- and concentration-dependent inhibition of IL-6 (JAK1/JAK2)-induced STAT3 and IL-7 (JAK1/JAK3)-induced STAT5 phosphorylation in whole blood. The maximal inhibition was observed 1 hour after dosing which returned to near baseline by the end of dosing interval.

### Lymphocytes

In patients with rheumatoid arthritis, treatment with upadacitinib was associated with a small, transient increase in mean ALC from baseline up to Week 36 which gradually returned to, at or near baseline levels with continued treatment.

### Immunoglobulins

In patients with rheumatoid arthritis, small decreases from baseline in mean IgG and IgM levels were observed with upadacitinib treatment in the controlled period; however, the mean values at baseline and at all visits were within the normal reference range.

### Cardiac Electrophysiology

At 6 times the mean maximum exposure of the 15 mg once daily dose, there was no clinically relevant effect on the QTc interval.

### Pharmacokinetics

Upadacitinib plasma exposures are proportional to dose over the therapeutic dose range. Steady-state plasma concentrations are achieved within 4 days with minimal accumulation after multiple once-daily administrations. Upadacitinib pharmacokinetics are similar between rheumatoid arthritis, psoriatic arthritis, and atopic dermatitis patients.

### Absorption

Following oral administration of upadacitinib extended-release formulation, upadacitinib is absorbed with a median Tₘₐₓ of 2 to 4 hours. Coadministration of upadacitinib with a high-fat/high-calorie meal had no clinically relevant effect on upadacitinib exposures (increased AUC_{inf} by 29% and Cₘₐₓ by 39%). In clinical trials, upadacitinib was administered without regard to meals.

### Distribution

Upadacitinib is 52% bound to plasma proteins. Upadacitinib partitions similarly between plasma and blood cellular components with a blood to plasma ratio of 1.0.

### Elimination

### Metabolism

Upadacitinib metabolism is mediated by mainly CYP3A4 with a potential minor contribution from CYP2D6. The pharmacologic activity of upadacitinib is attributed to the parent molecule. In a human radiolabeled study, unchanged upadacitinib accounted for 79% of the total radioactivity in plasma while the main metabolite detected (product of monooxidation followed by glucuronidation) accounted for 13% of the total plasma radioactivity. No active metabolites have been identified for upadacitinib.

### Excretion

Following single dose administration of [¹⁴C]-upadacitinib immediate-release solution, upadacitinib was eliminated predominantly as the unchanged parent substance in urine (24%) and feces (38%). Approximately 34% of upadacitinib dose was excreted as metabolites. Upadacitinib mean terminal elimination half-life ranged from 8 to 14 hours.

### Specific Populations

### Body Weight, Gender, Race, and Age

Body weight, gender, race, ethnicity, and age did not have a clinically meaningful effect on upadacitinib exposure.

Patients with Renal Impairment

Upadacitinib AUC_{inf} was 18%, 33%, and 44% higher in patients with mild, moderate, and severe renal impairment, respectively, compared to patients with normal renal function. Upadacitinib Cmax was similar in patients with normal and impaired renal function. Mild or moderate renal impairment has no clinically relevant effect on upadacitinib exposure for the 15 or 30 mg once daily dosing regimens. Severe renal impairment is likely to increase the systemic exposure of upadacitinib after 30 mg once daily dosing in patients with atopic dermatitis. This may increase the risk of adverse reactions; therefore, dosage modification in patients with severe renal impairment is recommended.

Patients with Hepatic Impairment

Mild (Child-Pugh A) and moderate (Child-Pugh B) hepatic impairment has no clinically relevant effect on upadacitinib exposure. Upadacitinib AUC_{inf} was 28% and 24% higher in patients with mild and moderate hepatic impairment, respectively, compared to patients with normal liver function. Upadacitinib Cₘₐₓ was unchanged in patients with mild hepatic impairment and 43% higher in patients with moderate hepatic impairment compared to patients with normal liver function. Upadacitinib was not studied in patients with severe hepatic impairment (Child-Pugh C).

### Drug Interaction Studies

### Potential for Other Drugs to Influence the Pharmacokinetics of Upadacitinib

Upadacitinib is metabolized in vitro by CYP3A4 with a minor contribution from CYP2D6. The effect of co-administered drugs on upadacitinib plasma exposures is provided in Table 4.

TABLE 4
Change in Pharmacokinetics of Upadacitinib in the Presence of Co-administered Drugs

| | Regimen of | Ratio (90% CI)^{a} | |
|---|---|---|---|
| Co-administered Drug | Co-administered Drug | Cₘₐₓ | AUC |
| Methotrexate | 10 to 25 mg/week | 0.97 (0.86-1.09) | 0.99 (0.93-1.06) |
| Strong CYP3A4 inhibitor: Ketoconazole | 400 mg once daily x 6 days | 1.70 (1.55-1.89) | 1.75 (1.62-1.88) |
| Strong CYP3A4 inducer: Rifampin | 600 mg once daily x 9 days | 0.49 (0.44-0.55) | 0.39 (0.37-0.42) |
| OATP1B inhibitor: Rifampin | 600 mg single dose | 1.14 (1.02-1.28) | 1.07 (1.01-1.14) |

CI: Confidence interval
^{a}Ratios for Cₘₐₓ and AUC compare co-administration of the medication with upadacitinib vs. administration of upadacitinib alone.

pH modifying medications (e.g., antacids or proton pump inhibitors) are not expected to affect upadacitinib plasma exposures based on in vitro assessments and population pharmacokinetic analyses. CYP2D6 metabolic phenotype had no effect on upadacitinib pharmacokinetics (based on population pharmacokinetic analyses), indicating that inhibitors of CYP2D6 have no clinically relevant effect on upadacitinib exposures.

### Potential for Upadacitinib to Influence the Pharmacokinetics of Other Drugs

In vitro studies indicate that upadacitinib does not inhibit or induce the activity of cytochrome P450 (CYP) enzymes (CYP1A2, CYP2B6, CYP2C8, CYP2C9, CYP2C19, CYP2D6, and CYP3A4) at clinically relevant concentrations. In vitro studies indicate that upadacitinib does not inhibit the transporters P-gp, BCRP, OATP1B1, OATP1B3, OCT1, OCT2, OAT1, OAT3, MATE1, and MATE2K at clinically relevant concentrations.

Clinical studies indicate that upadacitinib has no clinically relevant effects on the pharmacokinetics of co-administered drugs. Summary of results from clinical studies which evaluated the effect of upadacitinib on other drugs is provided in Table 5.

TABLE 5
Change in Pharmacokinetics of Co-administered Drugs or In Vivo
Markers of CYP Activity in the Presence of Upadacitinib

| Co-administered Drug | Multiple-Dose Regimen of | Ratio (90% CI)^{a} | |
|---|---|---|---|
| or CYP Activity Marker | Upadacitinib | Cₘₐₓ | AUC |
| Methotrexate | 6 mg to 24 mg BID^{b} | 1.03 (0.86-1.23) | 1.14 (0.91-1.43) |
| Sensitive CYP1A2 Substrate: Caffeine | 30 mg QD^{c} | 1.13 (1.05-1.22) | 1.22 (1.15-1.29) |
| Sensitive CYP3A Substrate: Midazolam | 30 mg QD^{c} | 0.74 (0.68-0.80) | 0.74 (0.68-0.80) |
| Sensitive CYP2D6 Substrate: Dextromethorphan | 30 mg QD^{c} | 1.09 (0.98-1.21) | 1.07 (0.95-1.22) |
| Sensitive CYP2C9 Substrate: S-Warfarin | 30 mg QD^{c} | 1.07 (1.02-1.11) | 1.11 (1.07-1.15) |
| Sensitive CYP2C19 Marker: 5-OH | 30 mg QD^{c} | — | 1.09 (1.00-1.19) |
| Omeprazole to Omeprazole metabolic ratio | | | |
| CYP2B6 Substrate: Bupropion | 30 mg QD^{c} | 0.87 (0.79-0.96) | 0.92 (0.87-0.98) |
| Rosuvastatin | 30 mg QD^{c} | 0.77 (0.63-0.94) | 0.67 (0.56-0.82) |
| Atorvastatin | 30 mg QD^{c} | 0.88 (0.79-0.97) | 0.77 (0.70-0.85) |
| Ethinylestradiol | 30 mg QD^{c} | 0.96 (0.89-1.02) | 1.11 (1.04-1.19) |
| Levonorgestrel | 30 mg QD^{c} | 0.96 (0.87-1.06) | 0.96 (0.85-1.07) |

CYP: cytochrome P450;
CI: Confidence interval;
BID: twice daily;
QD: once daily
^{a}Ratios for Cₘₐₓ and AUC compare co-administration of the medication with upadacitinib vs. administration of medication alone.
^{b}Immediate-release formulation
^{c}Extended-release formulation

### Nonclinical Toxicology

### Carcinogenesis, Mutagenesis, Impairment of Fertility

### Carcinogenesis

The carcinogenic potential of upadacitinib was evaluated in Sprague-Dawley rats and Tg.rasH2 mice. No evidence of tumorigenicity was observed in male or female rats that received upadacitinib for up to 101 weeks at oral doses up to 15 or 20 mg/kg/day, respectively (approximately 2.4 and 6.0 times the MRHD of 30 mg on an AUC basis, respectively). No evidence of tumorigenicity was observed in male or female Tg.rasH2 mice that received upadacitinib for 26 weeks at oral doses up to 20 mg/kg/day.

### Mutagenesis

Upadacitinib tested negatively in the following genotoxicity assays: the in vitro bacterial mutagenicity assay (Ames assay), in vitro chromosome aberration assay in human peripheral blood lymphocytes, and in vivo rat bone marrow micronucleus assay.

### Impairment of Fertility

Upadacitinib had no effect on fertility in male or female rats at oral doses up to 50 mg/kg/day in males and 75 mg/kg/day in females (approximately 24 and 48 times the MRHD of 30 mg in males and females, respectively, on an AUC basis). However, maintenance of pregnancy was adversely affected at oral doses of 25 mg/kg/day and 75 mg/kg/day based upon dose-related findings of increased post-implantation losses (increased resorptions) and decreased numbers of mean viable embryos per litter (approximately 13 and 48 times the MRHD of 30 mg on an AUC basis, respectively). The number of viable embryos was unaffected in female rats that received upadacitinib at an oral dose of 5 mg/kg/day and were mated to males that received the same dose (approximately 1.0 times the MRHD of 30 mg on an AUC basis).

### Clinical Studies

### Rheumatoid Arthritis

The efficacy and safety of RINVOQ® 15 mg once daily were assessed in five Phase 3 randomized, double-blind, multicenter trials in patients with moderately to severely active rheumatoid arthritis and fulfilling the ACR/EULAR 2010 classification criteria. Patients 18 years of age and older were eligible to participate. The presence of at least 6 tender and 6 swollen joints and evidence of systemic inflammation based on elevation of hsCRP was required at baseline. Although other doses have been studied, the recommended dosage of RINVOQ® is 15 mg once daily.

Trial RA-I (NCT02706873) was a 24-week monotherapy trial in 947 patients with moderately to severely active rheumatoid arthritis who were naïve to methotrexate (MTX). Patients received RINVOQ 15 mg or upadacitinib 30 mg orally once daily or MTX as monotherapy. At Week 26, non-responding patients on upadacitinib could be rescued with the addition of MTX, while patients on MTX could be rescued with the addition of blinded RINVOQ 15 mg or upadacitinib 30 mg once daily. The primary endpoint was the proportion of patients who achieved an ACR50 response at Week 12. Key secondary endpoints included disease activity score (DAS28-CRP) ≤3.2 at Week 12, DAS28-CRP <2.6 at Week 24, change from baseline in HAQ-DI at Week 12, and change from baseline in van der Heijde-modified total Sharp Score (mTSS) at Week 24.

Trial RA-II (NCT02706951) was a 14-week monotherapy trial in 648 patients with moderately to severely active rheumatoid arthritis who had an inadequate response to MTX. Patients received RINVOQ® 15 mg or upadacitinib 30 mg once daily monotherapy or continued their stable dose of MTX monotherapy. At Week 14, patients who were randomized to MTX were advanced to RINVOQ® 15 mg or upadacitinib 30 mg once daily monotherapy in a blinded manner based on pre-determined assignment at baseline. The primary endpoint was the proportion of patients who achieved an ACR20 response at Week 14. Key secondary endpoints included DAS28-CRP ≤3.2, DAS28-CRP <2.6, and change from baseline in HAQ-DI at Week 14.

Trial RA-III (NCT02675426) was a 12-week trial in 661 patients with moderately to severely active rheumatoid arthritis who had an inadequate response to conventional disease modifying anti-rheumatic drugs (cDMARDs). Patients received RINVOQ® 15 mg or upadacitinib 30 mg once daily or placebo added to background cDMARD therapy. At Week 12, patients who were randomized to placebo were advanced to RINVOQ® 15 mg or upadacitinib 30 mg once daily in a blinded manner based on pre-determined assignment at baseline. The primary endpoint was the proportion of patients who achieved an ACR20 response at Week 12. Key secondary endpoints included DAS28-CRP≤3.2, DAS28-CRP<2.6, and change from baseline in HAQ-DI at Week 12.

Trial RA-IV (NCT02629159) was a 48-week trial in 1629 patients with moderately to severely active rheumatoid arthritis who had an inadequate response to MTX. Patients received RINVOQ® 15 mg once daily, active comparator, or placebo added to background MTX. From Week 14, non-responding patients on RINVOQ® 15 mg could be rescued to active comparator in a blinded manner, and non-responding patients on active comparator or placebo could be rescued to RINVOQ® 15 mg in a blinded manner. At Week 26, all patients randomized to placebo were switched to RINVOQ® 15 mg once daily in a blinded manner. The primary endpoint was the proportion of patients who achieved an ACR20 response at Week 12 versus placebo. Key secondary endpoints versus placebo included DAS28-CRP≤3.2, DAS28-CRP<2.6, change from baseline in HAQ-DI at Week 12, and change from baseline in mTSS at Week 26.

Trial RA-V (NCT02706847) was a 12-week trial in 499 patients with moderately to severely active rheumatoid arthritis who had an inadequate response or intolerance to biologic DMARDs. Patients received RINVOQ® 15 mg or upadacitinib 30 mg once daily or placebo added to background cDMARD therapy. At Week 12, patients who were randomized to placebo were advanced to RINVOQ 15 mg or upadacitinib 30 mg once daily in a blinded manner based on pre-determined assignment at baseline. The primary endpoint was the proportion of patients who achieved an ACR20 response at Week 12. Key secondary endpoints included DAS28-CRP <3.2 and change from baseline in HAQ-DI at Week 12.

### Clinical Response

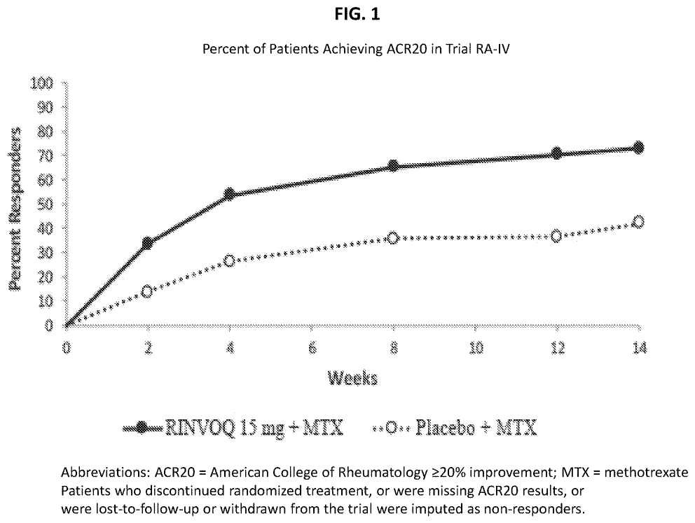

The percentages of RINVOQ®-treated patients achieving ACR20, ACR50, and ACR70 responses, and DAS28(CRP) <2.6 in all trials are shown in Table 6. Patients treated with RINVOQ® 15 mg, alone or in combination with cDMARDs, achieved higher ACR response rates compared to MTX monotherapy or placebo, respectively, at the primary efficacy timepoint (Table 6). In Trial IV, the percent of patients achieving ACR20 response by visit is shown in FIG. 1. In Trials RA-III and RA-V, higher ACR20 response rates were observed at 1 week with RINVOQ 15 mg versus placebo. Treatment with RINVOQ 15 mg, alone or in combination with cDMARDs, resulted in greater improvements in the ACR components compared to MTX or placebo at the primary efficacy timepoint (Table 7).

TABLE 6
Clinical Response in RA Patients in Trials RA-I, RA-II, RA-III, RA-IV and RA V

| | | | | | Trial RA-III | | Trial RA-IV | | Trial RA-V | |
|---|---|---|---|---|---|---|---|---|---|---|
| | Trial RA-I | | Trial RA-II | | cDMARD-IR | | MTX-IR | | bDMARD-IR | |
| | MTX-Naive | | MTX-IR | | Background | | Background | | Background | |
| | Monotherapy | | Monotherapy | | cDMARDs | | MTX | | cDMARDs | |
| | | RINVOQ | | RINVOQ | | RINVOQ | | RINVOQ | | RINVOQ |
| | | 15 mg | | 15 mg | | 15 mg | | 15 mg | | 15 mg |
| | | % | | % | | % | | % | | % |
| | MTX | Δ (95% CI) | MTX | Δ (95% CI) | PBO | Δ (95% CI) | PBO | Δ (95% CI) | PBO | Δ (95% CI) |
| N | 314 | 317 | 216 | 217 | 221 | 221 | 651 | 651 | 169 | 164 |

Week

ACR20

| | | | | | | | | | | |
|---|---|---|---|---|---|---|---|---|---|---|
| 12^{a}/14^{b} | 54 | 76 | 41 | 68 | 36 | 64 | 36 | 71 | 28 | 65 |
| | | 22 (14, 29) | | 26 (17, 36) | | 28 (19, 37) | | 34 (29, 39) | | 36 (26, 46) |
| 24^{c}/26^{d} | 59 | 79 | | | | | 36 | 67 | | |
| | | 20 (13, 27) | | | | | | 32 (27, 37) | | |

ACR50

| | | | | | | | | | | |
|---|---|---|---|---|---|---|---|---|---|---|
| 12^{a}/14^{b} | 28 | 52 | 15 | 42 | 15 | 38 | 15 | 45 | 12 | 34 |
| | | 24 (16, 31) | | 27 (18, 35) | | 23 (15, 31) | | 30 (26, 35) | | 22 (14, 31) |
| 24^{c}/26^{d} | 33 | 60 | | | | | 21 | 54 | | |
| | | 27 (19, 34) | | | | | | 33 (28, 38) | | |

ACR70

| | | | | | | | | | | |
|---|---|---|---|---|---|---|---|---|---|---|
| 12^{a}/14^{b} | 14 | 32 | 3 | 23 | 6 | 21 | 5 | 25 | 7 | 12 |
| | | 18 (12, 25) | | 20 (14, 26) | | 15 (9, 21) | | 20 (16, 24) | | 5 (−1, 11) |
| 24^{c}/26^{d} | 18 | 44 | | | | | 10 | 35 | | |
| | | 26 (19, 33) | | | | | | 25 (21, 29) | | |

DAS28-CRP <2.6

| | | | | | | | | | | |
|---|---|---|---|---|---|---|---|---|---|---|
| 12^{a}/14^{b} | 14 | 36 | 8 | 28 | 10 | 31 | 6 | 29 | 9 | 29 |
| | | 22 (15, 28) | | 20 (13, 27) | | 21 (14, 28) | | 23 (19, 27) | | 19 (11, 27) |
| 24^{c}/26^{d} | 18 | 48 | | | | | 9 | 41 | | |
| | | 30 (23, 37) | | | | | | 32 (27, 36) | | |

Abbreviations:
ACR20 (or 50 or 70) = American College of Rheumatology ≥20% (or ≥50% or ≥70%) improvement;
bDMARD = biologic disease-modifying anti-rheumatic drug;
CRP = c-reactive protein;
DAS28 = Disease Activity Score 28 joints;
cDMARDs = conventional disease-modifying anti-rheumatic drugs;
MTX = methotrexate;
PBO = placebo;
IR = inadequate responder
Patients who discontinued randomized treatment, or had cross-over between randomized treatments, or were missing data at week of evaluation were imputed as non-responders in the analyses.
^{a}Trial RA-I, Trial RA-III, Trial RA-IV, Trial RA-V
^{b}Trial RA-II
^{c}Trial RA-I
^{d}Trial RA-IV

TABLE 7
Components of ACR Response at Primary Efficacy Timepoint^{a}

| | | | | | Trial RA-III | | Trial RA-IV | | Trial RA-V | |
|---|---|---|---|---|---|---|---|---|---|---|
| | Trial RA-I | | Trial RA-II^{b} | | cDMARD-IR | | MTX-IR | | bDMARD-IR | |
| | MTX-Naive | | MTX-IR | | Background | | Background | | Background | |
| | Monotherapy | | Monotherapy | | cDMARDs | | MTX | | cDMARDs | |
| | | RINVOQ | | RINVOQ | | RINVOQ | | RINVOQ | | RINVOQ |
| | MTX | 15 mg | MTX | 15 mg | PBO | 15 mg | PBO | 15 mg | PBO | 15 mg |
| N | 314 | 317 | 216 | 217 | 221 | 221 | 651 | 651 | 169 | 164 |

Number of tender joints (0-68)

| | | | | | | | | | | |
|---|---|---|---|---|---|---|---|---|---|---|
| Baseline | 26 | 25 | 25 | 24 | 25 | 25 | 26 | 26 | 28 | 28 |
| | (16) | (14) | (16) | (15) | (15) | (14) | (14) | (15) | (15) | (16) |
| Week | 13 | 9 | 15 | 10 | 16 | 12 | 16 | 10 | 18 | 11 |
| 12/14 | (15) | (12) | (16) | (13) | (17) | (14) | (15) | (13) | (17) | (14) |

Number of swollen joints (0-66)

| | | | | | | | | | | |
|---|---|---|---|---|---|---|---|---|---|---|
| Baseline | 17 | 17 | 17 | 16 | 15 | 16 | 16 | 17 | 16 | 17 |
| | (11) | (10) | (12) | (11) | (9) | (10) | (9) | (10) | (10) | (11) |
| Week | 6 | 5 | 9 | 6 | 9 | 7 | 9 | 5 | 9 | 6 |
| 12/14 | (8) | (7) | (11) | (9) | (10) | (10) | (9) | (7) | (10) | (8) |

Pain^{c}

| | | | | | | | | | | |
|---|---|---|---|---|---|---|---|---|---|---|
| Baseline | 66 | 68 | 63 | 62 | 62 | 64 | 65 | 66 | 69 | 68 |
| | (21) | (21) | (21) | (23) | (21) | (19) | (21) | (21) | (21) | (20) |
| Week | 41 | 31 | 49 | 36 | 51 | 33 | 49 | 33 | 55 | 41 |
| 12/14 | (25) | (25) | (25) | (27) | (26) | (24) | (25) | (24) | (28) | (28) |

Patient global assessment^{c}

| | | | | | | | | | | |
|---|---|---|---|---|---|---|---|---|---|---|
| Baseline | 66 | 67 | 60 | 62 | 60 | 63 | 64 | 64 | 66 | 67 |
| | (21) | (22) | (22) | (22) | (20) | (22) | (21) | (22) | (23) | (20) |
| Week | 42 | 31 | 48 | 37 | 50 | 32 | 48 | 33 | 54 | 40 |
| 12/14 | (25) | (24) | (26) | (27) | (26) | (24) | (24) | (24) | (28) | (26) |

Disability Index (HAQ-DI)^{d}

| | | | | | | | | | | |
|---|---|---|---|---|---|---|---|---|---|---|
| Baseline | 1.60 | 1.60 | 1.47 | 1.47 | 1.42 | 1.48 | 1.61 | 1.63 | 1.56 | 1.67 |
| | (0.67) | (0.67) | (0.66) | (0.66) | (0.63) | (0.61) | (0.61) | (0.64) | (0.60) | (0.64) |
| Week | 1.08 | 0.76 | 1.19 | 0.86 | 1.13 | 0.85 | 1.28 | 0.98 | 1.33 | 1.24 |
| 12/14 | (0.72) | (0.69) | (0.69) | (0.67) | (0.70) | (0.66) | (0.67) | (0.68) | (0.66) | (0.77) |

Physician global assessment^{c}

| | | | | | | | | | | |
|---|---|---|---|---|---|---|---|---|---|---|
| Baseline | 69 | 67 | 62 | 66 | 64 | 64 | 66 | 66 | 67 | 69 |
| | (16) | (17) | (17) | (18) | (18) | (16) | (18) | (17) | (17) | (17) |
| Week | 32 | 22 | 37 | 26 | 41 | 26 | 41 | 27 | 39 | 29 |
| 12/14 | (22) | (19) | (24) | (21) | (24) | (21) | (25) | (21) | (25) | (22) |

CRP (mg/L)

| | | | | | | | | | | |
|---|---|---|---|---|---|---|---|---|---|---|
| Baseline | 21.2 | 23.0 | 14.5 | 14.0 | 12.6 | 16.6 | 18.0 | 17.9 | 16.3 | 16.3 |
| | (22.1) | (27.4) | (17.3) | (16.5) | (14.0) | (19.2) | (21.5) | (22.5) | (21.1) | (18.6) |
| Week | 10.9 | 4.2 | 12.8 | 3.7 | 13.1 | 4.6 | 16.2 | 5.5 | 13.9 | 5.0 |
| 12/14 | (14.9) | (8.8) | (21.4) | (7.8) | (15.5) | (9.6) | (19.8) | (10.9) | (17.3) | (14.0) |

Abbreviations:
ACR = American College of Rheumatology;
bDMARD = biologic disease-modifying anti-rheumatic drug;
CRP = c-reactive protein;
cDMARDs = conventional disease-modifying anti-rheumatic drugs;
HAQ-DI = Health Assessment Questionnaire Disability Index;
IR = inadequate responder;
MTX = methotrexate;
PBO = placebo
^{a}Data shown are mean (standard deviation).
^{b}Primary efficacy timepoint is at Week 14.
^{c}Visual analog scale: 0 = best, 100 = worst.
^{d}Health Assessment Questionnaire-Disability Index: 0 = best, 3 = worst; 20 questions; 8 categories: dressing and grooming, arising, eating, walking, hygiene, reach, grip, and activities.

In RA-I and RA-IV, a higher proportion of patients treated with RINVOQ 15 mg alone or in combination with MTX, achieved DAS28-CRP <2.6 compared to MTX or placebo at the primary efficacy timepoint (Table 8).

TABLE 8
Proportion of Patients with DAS28-CRP Less Than 2.6 with Number
of Residual Active Joints at Primary Efficacy Timepoint

| | Trial RA-I MTX-Naive | | |
|---|---|---|---|
| | Monotherapy | | |
| | MTX | | RINVOQ 15 mg |
| DAS28-CRP Less Than 2.6 | N = 314 | | N = 317 |
| Proportion of responders at Week 12 (n) | 14% | (43) | 36% (113) |
| Of responders, proportion with 0 active joints (n) | 51% | (22) | 45% (51) |
| Of responders, proportion with 1 active joint (n) | 35% | (15) | 23% (26) |
| Of responders, proportion with 2 active joints (n) | 9% | (4) | 17% (19) |
| Of responders, proportion with 3 or more active joints (n) | 5% | (2) | 15% (17) |
| | Trial RA-IV MTX-IR | | |
| | Background MTX | | |
| | PBO | | RINVOQ 15 mg |
| DAS28-CRP Less Than 2.6 | N = 651 | | N = 651 |
| Proportion of responders at Week 12 (n) | 6% | (40) | 29% (187) |
| Of responders, proportion with 0 active joints (n) | 60% | (24) | 48% (89) |
| Of responders, proportion with 1 active joint (n) | 20% | (8) | 23% (43) |
| Of responders, proportion with 2 active joints (n) | 15% | (6) | 13% (25) |
| Of responders, proportion with 3 or more active joints (n) | 5% | (2) | 16% (30) |

Abbreviations:
CRP = c-reactive protein;
DAS28 = Disease Activity Score 28 joints;
MTX = methotrexate;
PBO = placebo;
IR = inadequate responder

### Radiographic Response

Inhibition of progression of structural joint damage was assessed using the modified Total Sharp Score (mTSS) and its components, the erosion score and joint space narrowing score, at Week 26 in Trial RA-IV and Week 24 in Trial RA-I. The proportion of patients with no radiographic progression (mTSS change from baseline <0) was also assessed.

In Trial RA-IV, treatment with RINVOQ® 15 mg inhibited the progression of structural joint damage compared to placebo in combination with cDMARDs at Week 26 (Table 9). Analyses of erosion and joint space narrowing scores were consistent with overall results.

In the placebo plus MTX group, 76% of the patients experienced no radiographic progression at Week 26 compared to 83% of the patients treated with RINVOQ® 15 mg.

In Trial RA-I, treatment with RINVOQ® 15 mg monotherapy inhibited the progression of structural joint damage compared to MTX monotherapy at Week 24 (Table 9). Analyses of erosion and joint space narrowing scores were consistent with overall results.

In the MTX monotherapy group, 78% of the patients experienced no radiographic progression at Week 24 compared to 87% of the patients treated with RINVOQ® 15 mg monotherapy.

TABLE 9
Radiographic Changes

| | Trial RA-IV MTX-IR | | |
|---|---|---|---|
| | Background MTX | | |
| | PBO | RINVOQ 15 mg | Estimated Difference |
| | (N = 651) | (N = 651) | vs PBO at Week 26 |
| mTSS | Mean (SD) | Mean (SD) | (95% CI)^{a} |
| Baseline | 35.9 (52) | 34.0 (50) | |
| Week 26^{b} | 0.78 (0.1) | 0.15 (0.1) | −0.63 (−0.92, −0.34) |
| | Trial RA-I MTX-naïve | | |
| | Monotherapy | | |
| | MTX | RINVOQ 15 mg | Estimated Difference |
| | (N = 309) | (N = 309) | vs MTX at Week 24 |
| | Mean (SD) | Mean (SD) | (95% CI)^{c} |
| Baseline | 13.3 (31) | 18.1 (38) | |
| Week 24^{d} | 0.67 (2.8) | 0.14 (1.4) | −0.53 (−0.85, −0.20) |

Abbreviations:
mTSS = modified Total Sharp Score,
MTX = methotrexate;
PBO = placebo;
SD = standard deviation;
IR = inadequate responders;
bDMARDs = biologic disease modifying anti-rheumatic drugs;
LS = least squares;
CI = confidence intervals
^{a}LS means and 95% CI based on a random coefficient model fit to the mTSS value adjusting for time, treatment group, prior bDMARDs use, treatment group-by-time interaction, with random slopes and random intercept.
^{b}Estimated linear rate of structural progression by Week 26 and standard errors are presented.
^{c}LS means and 95% CI based on a linear regression model fit to change from baseline in mTSS adjusting for treatment group, baseline mTSS, and geographic region.
^{d}Mean change from baseline and standard deviation are presented.

### Physical Function Response

Treatment with RINVOQ® 15 mg, alone or in combination with cDMARDs, resulted in a greater improvement in physical function at Week 12/14 compared to all comparators as measured by HAQ-DI.

### Other Health-Related Outcomes

In all trials except for Trial RA-V, patients receiving RINVOQ® 15 mg had greater improvement from baseline in physical component summary (PCS) score, mental component summary (MCS) scores, and in all 8 domains of the Short Form Health Survey (SF-36) compared to placebo in combination with cDMARDs or MTX monotherapy at Week 12/14.

Fatigue was assessed by the Functional Assessment of Chronic Illness Therapy-Fatigue score (FACIT-F) in Trials RA-I, RA-III, and RA-IV. Improvement in fatigue at Week 12 was observed in patients treated with RINVOQ 15 mg compared to patients on placebo in combination with cDMARDs or MTX monotherapy.

### 14.2 Psoriatic Arthritis

The efficacy and safety of RINVOQ® 15 mg once daily were assessed in two Phase 3 randomized, double-blind, multicenter, placebo-controlled studies in patients 18 years of age or older with moderately to severely active psoriatic arthritis. All patients had active psoriatic arthritis for at least 6 months based upon the Classification Criteria for Psoriatic Arthritis (CASPAR), at least 3 tender joints and at least 3 swollen joints, and active plaque psoriasis or history of plaque psoriasis. Although another dose has been studied, the recommended dose of RINVOQ is 15 mg once daily for psoriatic arthritis.

Study PsA-I (NCT03104400) was a 24-week trial in 1705 patients with moderately to severely active psoriatic arthritis who had an inadequate response or intolerance to at least one non-biologic DMARD. Patients received RINVOQ® 15 mg or upadacitinib 30 mg once daily, adalimumab, or placebo, alone or in combination with background non-biologic DMARDs. At Week 24, all patients randomized to placebo were switched to RINVOQ 15 mg or upadacitinib 30 mg once daily in a blinded manner. The primary endpoint was the proportion of patients who achieved an ACR20 response at Week 12.

Study PsA-II (NCT03104374) was a 24-week trial in 642 patients with moderately to severely active psoriatic arthritis who had an inadequate response or intolerance to at least one biologic DMARD. Patients received RINVOQ® 15 mg or upadacitinib 30 mg once daily or placebo, alone or in combination with background non-biologic DMARDs. At Week 24, all patients randomized to placebo were switched to RINVOQ® 15 mg or upadacitinib 30 mg once daily in a blinded manner. The primary endpoint was the proportion of patients who achieved an ACR20 response at Week 12.

### Clinical Response

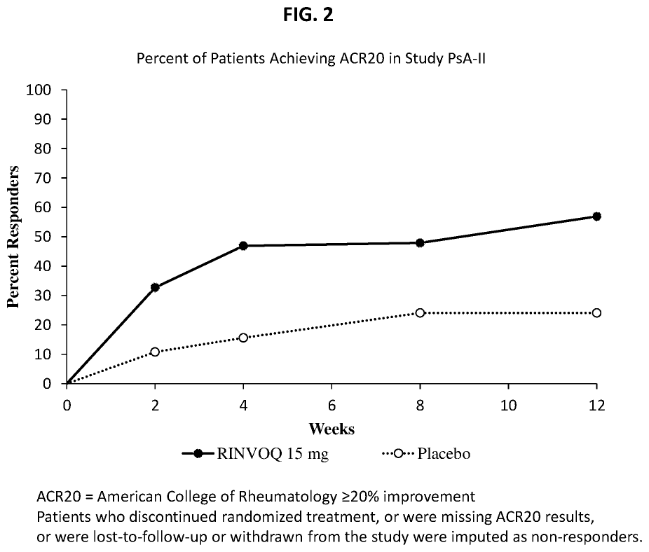

In both studies, patients treated with RINVOQ® 15 mg achieved significantly higher ACR20 responses compared to placebo at Week 12 (Table 10, FIG. 2). A higher proportion of patients treated with RINVOQ 15 mg achieved ACR50 and ACR70 responses at Week 12 compared to placebo. Treatment with RINVOQ 15 mg resulted in improvements in the ACR components compared to placebo at the primary efficacy time point (Table 11).

TABLE 10
Clinical Response

| Study | | | |
|---|---|---|---|
| Study PsA-I | | Study PsA-II | |
| non-biologic DMARD-IR | | bDMARD-IR | |
| Treatment Group | | | |
| PBO | RINVOQ 15 mg | PBO | RINVOQ 15 mg |
| % | % Δ (95% CI) | % | % Δ (95% CI) |
| N | | | |
| 423 | 429 | 212 | 211 |

ACR20

| | | | | |
|---|---|---|---|---|
| Week 12 | 36 | 71 | 24 | 57 |
| | | 35 (28, 41) | | 33 (24, 42) |

ACR50

| | | | | |
|---|---|---|---|---|
| Week 12 | 13 | 38 | 5 | 32 |
| | | 24 (19, 30) | | 27 (20, 34) |

ACR70

| | | | | |
|---|---|---|---|---|
| Week 12 | 2 | 16 | 1 | 9 |
| | | 13 (10, 17) | | 8 (4, 12) |

Abbreviations:
ACR20 (or 50 or 70) = American College of Rheumatology ≥20% (or ≥50% or ≥70%) improvement,
bDMARD = biologic disease-modifying anti-rheumatic drug;
IR = inadequate responder;
PBO = placebo
Patients who discontinued randomized treatment or were missing data at week of evaluation were imputed as non-responders in the analyses.

TABLE 11
Components of ACR Response^{a}

| Study | | | |
|---|---|---|---|
| Study PsA-I | | Study PsA-II | |
| non-biologic DMARD-IR | | bDMARD-IR | |
| Treatment Group | | | |
| PBO | RINVOQ 15 mg | PBO | RINVOQ 15 mg |
| (N = 423) | (N = 429) | (N = 212) | (N = 211) |

Number of tender/painful joints (0-68)

| | | | | | | | | |
|---|---|---|---|---|---|---|---|---|
| Baseline | 20.0 | (14.3) | 20.4 | (14.7) | 25.3 | (17.6) | 24.9 | (17.3) |
| Week 12 | 12.5 | (13.3) | 8.8 | (12.5) | 19.3 | (18.5) | 12.6 | (15.6) |

Number of swollen joints (0-66)

| | | | | | | | | |
|---|---|---|---|---|---|---|---|---|
| Baseline | 11.0 | (8.2) | 11.6 | (9.3) | 12.0 | (8.9) | 11.3 | (8.2) |
| Week 12 | 5.6 | (7.2) | 3.5 | (6.0) | 7.3 | (9.4) | 4.4 | (5.7) |

Patient assessment of pain^{b}

| | | | | | | | | |
|---|---|---|---|---|---|---|---|---|
| Baseline | 6.1 | (2.1) | 6.2 | (2.1) | 6.6 | (2.1) | 6.4 | (2.1) |
| Week 12 | 5.1 | (2.3) | 3.8 | (2.4) | 5.9 | (2.3) | 4.4 | (2.5) |

Patient global assessment^{b}

| | | | | | | | | |
|---|---|---|---|---|---|---|---|---|
| Baseline | 6.3 | (2.0) | 6.6 | (2.0) | 6.8 | (2.0) | 6.8 | (1.9) |
| Week 12 | 5.2 | (2.2) | 3.8 | (2.3) | 6.1 | (2.3) | 4.5 | (2.5) |

Disability index (HAQ-DI)^{c}

| | | | | | | | | |
|---|---|---|---|---|---|---|---|---|
| Baseline | 1.1 | (0.6) | 1.2 | (0.7) | 1.2 | (0.7) | 1.1 | (0.6) |
| Week 12 | 1.0 | (0.7) | 0.7 | (0.6) | 1.1 | (0.6) | 0.8 | (0.7) |

Physician global assessment^{b}

| | | | | | | | | |
|---|---|---|---|---|---|---|---|---|
| Baseline | 6.5 | (1.6) | 6.7 | (1.6) | 6.5 | (1.8) | 6.5 | (1.8) |
| Week 12 | 4.3 | (2.2) | 3.1 | (2.0) | 5.0 | (2.2) | 3.4 | (2.1) |

hsCRP (mg/L)

| | | | | | | | | |
|---|---|---|---|---|---|---|---|---|
| Baseline | 11.5 | (15.8) | 11.0 | (14.9) | 10.4 | (18.5) | 11.2 | (18.6) |
| Week 12 | 10.1 | (15.2) | 4.2 | (9.9) | 9.4 | (13.4) | 4.3 | (7.9) |

Abbreviations:
ACR = American College of Rheumatology;
hsCRP = high sensitivity c-reactive protein;
HAQ-DI = Health Assessment Questionnaire-Disability Index;
IR = inadequate responder;
PBO = placebo
^{a}Data shown are mean (standard deviation).
^{b}Numeric rating scale (NRS): 0 = best, 10 = worst
^{c}Health Assessment Questionnaire-Disability Index: 0 = best, 3 = worst; 20 questions; 8 categories: dressing and grooming, arising, eating, walking, hygiene, reach, grip, and activities.

The percentage of patients achieving ACR20 response by visit is shown in FIG. 2.

Treatment with RINVOQ® 15 mg resulted in improvement in dactylitis and enthesitis in patients with pre-existing dactylitis or enthesitis.

Treatment with RINVOQ® 15 mg resulted in improvement in skin manifestations in patients with PsA. However, RINVOQ® has not been studied in and is not indicated for the treatment of plaque psoriasis.

### Physical Function Response

In both studies, patients treated with RINVOQ® 15 mg showed significant improvement in physical function from baseline compared to placebo as assessed by HAQ-DI at Week 12 (Table 10). The mean difference (95% CI) from placebo in HAQ-DI change from baseline at Week 12 was −0.28 (−0.35, −0.22) in Study PsA-I and −0.21 (−0.30, −0.12) in Study PsA-II.

The proportion of HAQ-DI responders (>0.35 improvement from baseline in HAQ-DI score) at Week 12 in Study PsA-I and Study PsA-II was 58% and 45%, respectively, in patients receiving RINVOQ® 15 mg and 33% and 27%, respectively, in patients receiving placebo.

### Radiographic Response

In Study PsA-I, inhibition of progression of structural damage was assessed radiographically and expressed as the change from baseline in modified Total Sharp Score (mTSS) and its components, the erosion score and the joint space narrowing score, at Week 24.

Treatment with RINVOQ® 15 mg inhibited progression of structural joint damage compared to placebo at Week 24 (Table 12). Analyses of erosion and joint space narrowing scores were consistent with overall results. The proportion of patients with no radiographic progression (mTSS change ≤0) at Week 24 was 93% in patients receiving RINVOQ® 15 mg and 89% in patients receiving placebo.

TABLE 12
Radiographic Changes in Study PsA-I

| | | |
|---|---|---|
| PBO | RINVOQ 15 mg | Estimated Difference |
| (N = 392) | (N = 407) | vs PBO at Week 24 |
| Mean (SD) | Mean (SD) | (95% CI)^{a} |

mTSS

| | | | |
|---|---|---|---|
| Baseline | 13.32 (31.2) | 13.14 (42.4) | |
| Week 24^{b} | 0.23 (0.07) | −0.02 (0.04) | −0.25 (−0.41, −0.09) |

Abbreviations:
CI = confidence intervals;
LS = least squares;
mTSS = modified Total Sharp Score;
PBO = placebo;
SD = standard deviation
^{a}LS means and 95% CI based on a random coefficient model fit to the mTSS value adjusting for time, treatment group, current DMARD use (yes/no), treatment group-by-time interaction, with random slopes and random intercept.
^{b}Estimated linear rate of structural progression by Week 24 and standard errors are presented.

### Other Health-Related Outcomes

Health-related quality of life was assessed by SF-36. In both studies, patients receiving RINVOQ® 15 mg experienced significantly greater improvement from baseline in the Physical Component Summary score compared to placebo at Week 12. Greater improvement was also observed in the Mental Component Summary score and all 8 domains of SF-36 compared to placebo.

Patients receiving RINVOQ® 15 mg showed greater improvement from baseline in fatigue, as measured by FACIT-F score, at Week 12 compared to placebo in both studies.

### Atopic Dermatitis

The efficacy of RINVOQ® 15 mg and 30 mg once daily, was assessed in three Phase 3 randomized, double-blind, multicenter trials (AD-1, AD-2, AD-3; NCT03569293, NCT03607422, and NCT03568318, respectively) in a total of 2584 patients (12 years of age and older). RINVOQ® was evaluated in 344 pediatric patients and 2240 adult patients with moderate to severe atopic dermatitis (AD) not adequately controlled by topical medication(s).

Disease severity at baseline was defined by a validated Investigator's Global Assessment (vIGA-AD) score ≥3 in the overall assessment of AD on a severity scale of 0 to 4, an Eczema Area and Severity Index (EASI) score ≥16, a minimum body surface area (BSA) involvement of ≥10%, and weekly average Worst Pruritus Numerical Rating Scale (NRS) score ≥4. Overall, 57% of the patients were male and 69% were white. The mean age at baseline was 34 years (ranged from 12 to 75 years) and 13% of the patients were 12 to less than 18 years. At baseline, 49% of patients had a vIGA-AD score of 3 (moderate AD), and 51% of patients had a vIGA-AD score of 4 (severe AD). The baseline mean EASI score was 29 and the baseline weekly average Worst Pruritus NRS score was 7. Approximately 52% of the patients had prior exposure to systemic AD treatment.

In all three trials, patients received RINVOQ® once daily oral doses of 15 mg, 30 mg, or matching placebo for 16 weeks. In Trial AD-3, patients also received RINVOQ or placebo with concomitant topical corticosteroids (TCS) for 16 weeks.

All three trials assessed the co-primary endpoints of the proportion of patients with a vIGA-AD score of 0 (clear) or 1 (almost clear) with at least a 2-point improvement and the proportion of patients with EASI-75 (improvement of at least 75% in EASI score from baseline) at Week 16. Secondary endpoints included EASI-90 and EASI-100 at Week 16, and the proportion of patients with reduction in itch (>4-point improvement from baseline in the Worst Pruritus NRS) at Weeks 1, 4, and 16. In Trials AD-1 and AD-2, the proportion of patients with reduction in pain (>4-point improvement in the Atopic Dermatitis Symptom Scale [ADerm-SS] Skin Pain NRS) from baseline to Week 16 was a secondary endpoint.

### Clinical Response

### Monotherapy Trials (AD-1 and AD-2)

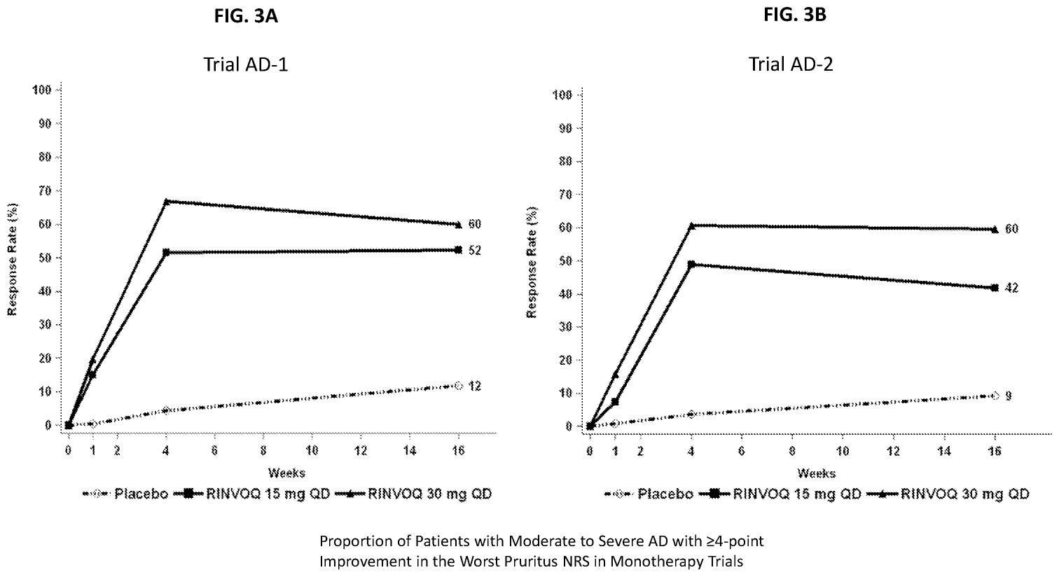

The results of RINVOQ® monotherapy trials (AD-1 and AD-2) are presented in Table 13. FIGS. 3A and 3B, respectively, present the proportion of patients with ≥4-point improvement in Worst Pruritus NRS at Weeks 1, 4, and 16 for Trials AD-1 and AD-2.

TABLE 13
Efficacy Results of Monotherapy Trials at Week 16 in Patients with Moderate to
Severe AD

| | Trial AD-1 | | | Trial AD-2 | | |
|---|---|---|---|---|---|---|
| | | RINVOQ | RINVOQ | | RINVOQ | RINVOQ |
| | PBO | 15 mg | 30 mg | PBO | 15 mg | 30 mg |
| Number of patients | 281 | 281 | 285 | 278 | 276 | 282 |
| randomized | | | | | | |
| vIGA-AD 0/1^{a,b} | 8% | 48% | 62% | 5% | 39% | 52% |
| Difference from | | 40% | 54% | | 34% | 47% |
| PBO (95% CI) | | (33%, 46%) | (47%, 60%) | | (28%, 40%) | (41%, 54%) |
| EASI-75^{a} | 16% | 70% | 80% | 13% | 60% | 73% |
| Difference from | | 53% | 63% | | 47% | 60% |
| PBO (95% CI) | | (46%, 60%) | (57%, 70%) | | (40%, 54%) | (53%, 66%) |
| EASI-90^{a} | 8% | 53% | 66% | 5% | 42% | 58% |
| Difference from | | 45% | 58% | | 37% | 53% |
| PBO (95% CI) | | (39%, 52%) | (51%, 64%) | | (31%, 43%) | (47%, 59%) |
| EASI-100^{a} | 2% | 17% | 27% | 1% | 14% | 19% |
| Difference from | | 15% | 25% | | 13% | 18% |
| PBO (95% CI) | | (10%, 20%) | (20%, 31%) | | (9%, 18%) | (13%, 23%) |
| Number of patients | 272 | 274 | 280 | 274 | 270 | 280 |
| with baseline Worst | | | | | | |
| Pruritus NRS score ≥ 4 | | | | | | |
| ≥4-point improvement | 12% | 52% | 60% | 9% | 42% | 60% |
| in Worst Pruritus NRS^{c} | | | | | | |
| Difference from | | 40% | 48% | | 33% | 50% |
| PBO (95% CI) | | (33%, 48%) | (41%, 55%) | | (26%, 39%) | (44%, 57%) |
| Number of patients | 233 | 237 | 249 | 247 | 237 | 238 |
| with baseline ADerm- | | | | | | |
| SS Skin Pain NRS | | | | | | |
| score ≥ 4 | | | | | | |
| ≥4-point improvement | 15% | 54% | 63% | 13% | 49% | 65% |
| in ADerm-SS Skin Pain | | | | | | |
| NRS^{d} | | | | | | |
| Difference from | | 39% | 49% | | 36% | 52% |
| PBO (95% CI) | | (31%, 47%) | (41%, 56%) | | (28%, 43%) | (44%, 59%) |

Abbreviations:
ADerm-SS = Atopic Dermatitis Symptom Scale;
PBO = placebo
^{a}Based on number of patients randomized
^{b}Responder was defined as a patient with vIGA-AD 0 or 1 (“clear” or “almost clear”) with a reduction of ≥2 points on a 0-4 ordinal scale
^{c}Based on number of patients whose baseline Worst Pruritus NRS is ≥4
^{d}Based on number of patients whose baseline ADerm-SS Skin Pain NRS is ≥4

Examination of age, gender, race, weight, and prior systemic treatment with immunosuppressants did not identify differences in response to RINVOQ® among these subgroups in Trials AD-1 and AD-2.

### Concomitant TCS Trial (AD-3)

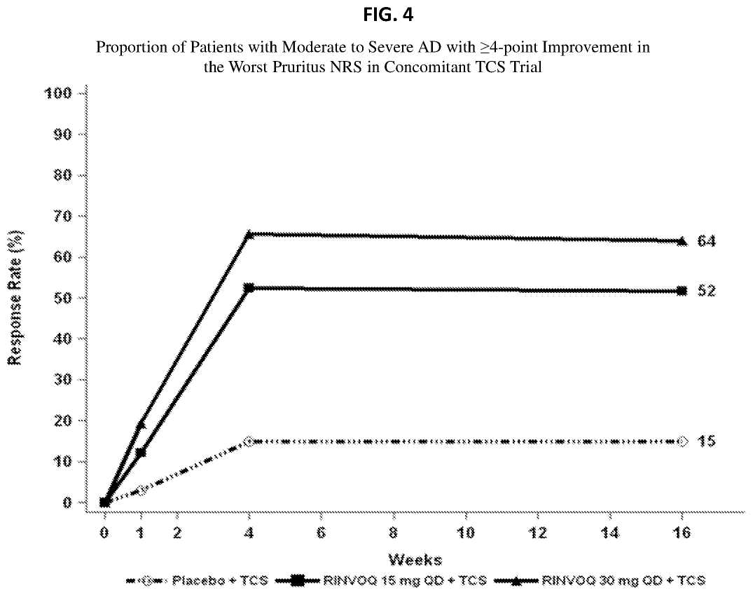

The results of the RINVOQ® with concomitant TCS trial (AD-3) are presented in Table 14. FIG. 4 presents the proportion of patients with ≥4-point improvement in Worst Pruritus NRS at Weeks 1, 4, and 16 for Trial AD-3. Examination of age, gender, race, weight, and prior systemic treatment with immunosuppressants did not identify differences in response to RINVOQ® among these subgroups in Trial AD-3.

TABLE 14
Efficacy Results with Concomitant TCS at Week
16 in Patients with Moderate to Severe AD

| | Trial AD-3 | | |
|---|---|---|---|
| | | RINVOQ | RINVOQ |
| | PBO + TCS | 15 mg + TCS | 30 mg + TCS |
| Number of patients | 304 | 300 | 297 |
| randomized | | | |
| vIGA-AD 0/1^{a, b} | 11% | 40% | 59% |
| Difference from | | 29% | 48% |
| PBO (95% CI) | | (22%, 35%) | (41%, 54%) |
| EASI-75^{a,} | 26% | 65% | 77% |
| Difference from | | 38% | 51% |
| PBO (95% CI) | | (31%, 45%) | (44%, 57%) |
| EASI-90^{a} | 13% | 43% | 63% |
| Difference from | | 30% | 50% |
| PBO (95% CI) | | (23%, 36%) | (43%, 56%) |
| EASI-100^{a} | 1% | 12% | 23% |
| Difference from | | 11% | 21% |
| PBO (95% CI) | | (7%, 14%) | (16%, 26%) |
| Number of patients with | 294 | 288 | 291 |
| baseline Worst Pruritus | | | |
| NRS score ≥4 | | | |
| ≥4-point improvement in | 15% | 52% | 64% |
| Worst Pruritus NRS^{c} | | | |
| Difference from | | 37% | 49% |
| PBO (95% CI) | | (30%, 44%) | (42%, 56%) |

Abbreviations:
PBO = placebo
^{a}Based on number of patients randomized
^{b}Responder was defined as a patient with vIGA-AD 0 or 1 (“clear” or “almost clear”) with a reduction of ≥2 points on a 0-4 ordinal scale
^{c}Based on number of patients whose baseline Worst Pruritus NRS is ≥4

### Pediatric Patient Population

The efficacy results of the RINVOQ® monotherapy trials (AD-1 and AD-2) and the RINVOQ® with concomitant TCS trial (AD-3) at Week 16 for pediatric patients 12 years of age and older are presented in Table 15 and Table 16, respectively.

TABLE 15
Efficacy Results of Monotherapy Trials for Pediatric Patients 12 Years of Age and
Older with Moderate to Severe AD at Week 16

| | Trial AD-1 | | | Trial AD-2 | | |
|---|---|---|---|---|---|---|
| | | RINVOQ | RINVOQ | | RINVOQ | RINVOQ |
| | PBO | 15 mg | 30 mg | PBO | 15 mg | 30 mg |
| Number of pediatric | 40 | 42 | 42 | 36 | 33 | 35 |
| patients randomized | | | | | | |
| vIGA-AD 0/1^{a,b} | 8% | 38% | 69% | 3% | 42% | 62% |
| Difference from | | 31% | 62% | | 40% | 60% |
| PBO (95% CI) | | (14%, 47%) | (45%, 78%) | | (22%, 57%) | (42%, 77%) |
| EASI-75^{a} | 8% | 71% | 83% | 14% | 67% | 74% |
| Difference from | | 63% | 75% | | 53% | 61% |
| PBO (95% CI) | | (47%, 79%) | (61%, 89%) | | (33%, 72%) | (42%, 79%) |
| Number of pediatric | 39 | 40 | 42 | 36 | 30 | 34 |
| patients with baseline | | | | | | |
| Worst Pruritus NRS | | | | | | |
| score ≥ 4 | | | | | | |
| ≥4-point improvement | 15% | 45% | 55% | 3% | 33% | 50% |
| in Worst Pruritus NRS^{c} | | | | | | |
| Difference from | | 30% | 39% | | 31% | 47% |
| PBO (95% CI) | | (10%, 49%) | (21%, 58%) | | (13%, 48%) | (30%, 65%) |

Abbreviations:
PBO = placebo
^{a}Based on number of pediatric patients randomized
^{b}Responder was defined as a patient with vIGA-AD 0 or 1 (“clear” or “almost clear”) with a reduction of ≥2 points on a 0-4 ordinal scale
^{c}Based on number of pediatric patients whose baseline Worst Pruritus NRS is ≥4

TABLE 16
Efficacy Results with Concomitant TCS for Pediatric Patients 12
Years of Age and Older with Moderate to Severe AD at Week 16

| | Trial AD-3 | | |
|---|---|---|---|
| | | RINVOQ | RINVOQ |
| | PBO + TCS | 15 mg + TCS | 30 mg + TCS |
| Number of pediatric | 40 | 39 | 37 |
| patients randomized | | | |
| vIGA-AD 0/1^{a, b} | 8% | 31% | 65% |
| Difference from | | 23% | 57% |
| PBO (95% CI) | | (7%, 40%) | (40%, 75%) |
| EASI-75^{a} | 30% | 56% | 76% |
| Difference from | | 26% | 46% |
| PBO (95% CI) | | (5%, 47%) | (26%, 65%) |
| Number of pediatric | 38 | 36 | 33 |
| patients with | | | |
| baseline Worst Pruritus | | | |
| NRS score ≥4 | | | |
| ≥4-point improvement in | 13% | 42% | 55% |
| Worst Pruritus NRS^{c} | | | |
| Difference from | | 29% | 41% |
| PBO (95% CI) | | (9%, 48%) | (21%, 61%) |

Abbreviations:
PBO = placebo
^{a}Based on number of pediatric patients randomized
^{b}Responder was defined as a patient with vIGA-AD 0 or 1 (“clear” or “almost clear”) with a reduction of ≥2 points on a 0-4 ordinal scale
^{c}Based on number of pediatric patients whose baseline Worst Pruritus NRS is ≥4

### How Supplied/Storage and Handling

### How Supplied

RINVOQ® extended-release tablets are supplied as:
15 mg: purple, biconvex oblong, with dimensions of 14×8 mm, and debossed with ‘a15’ on one side.
30 tablets in a bottle; NDC: 0074-2306-30
30 mg: red, biconvex oblong, with dimensions of 14×8 mm, and debossed with ‘a30’ on one side.
30 tablets in a bottle; NDC: 0074-2310-30

### Storage and Handling

Store at 2° C. to 25° C. (36° F. to 77° F.).

Store in the original bottle in order to protect from moisture.

### Patient Counseling Information

Advise the patient to read the FDA-approved patient labeling.

### Serious Infections

Inform patients that they may be more likely to develop infections when taking RINVOQ®. Instruct patients to contact their healthcare provider immediately during treatment if they develop any signs or symptoms of an infection. Advise patients that the risk of herpes zoster is increased in patients taking RINVOQ® and in some cases can be serious.

### Malignancies

Inform patients that RINVOQ® may increase their risk of certain cancers and that periodic skin examinations should be performed while using RINVOQ®. Advise patients that exposure to sunlight and UV light should be limited by wearing protective clothing and using a broad-spectrum sunscreen.

### Major Adverse Cardiovascular Events

Inform patients that RINVOQ® may increase their risk of major adverse cardiovascular events (MACE) including myocardial infarction, stroke, and cardiovascular death. Instruct all patients, especially current or past smokers or patients with other cardiovascular risk factors, to be alert for the development of signs and symptoms of cardiovascular events.

### Thrombosis

Inform patients that events of deep venous thrombosis and pulmonary embolism have been reported in clinical trials with RINVOQ®. Instruct patients to seek immediate medical attention if they develop any signs or symptoms of a DVT or PE.

### Hypersensitivity Reactions

Advise patients to discontinue RINVOQ® and seek immediate medical attention if they develop any signs and symptoms of allergic reactions.

### Gastrointestinal Perforations

Inform patients that gastrointestinal perforations have been reported in clinical trials with RINVOQ® and that risk factors include the use of NSAIDS or history of diverticulitis. Instruct patients to seek medical care immediately if they experience new onset of abdominal pain, fever, chills, nausea, or vomiting.

### Retinal Detachment

Inform patients that retinal detachment has been reported in clinical trials with RINVOQ®. Advise patients to immediately inform their healthcare provider if they develop any sudden changes in vision while receiving RINVOQ®.

### Laboratory Abnormalities

Inform patients that RINVOQ® may affect certain lab tests, and that blood tests are required before and during RINVOQ® treatment.

### Vaccinations

Advise patients to avoid use of live vaccines with RINVOQ®. Instruct patients to inform their healthcare practitioner that they are taking RINVOQ® prior to a potential vaccination.

### Embryo-Fetal Toxicity

Advise pregnant women and females of reproductive potential that exposure to RINVOQ® during pregnancy may result in fetal harm. Advise females to inform their healthcare provider of a known or suspected pregnancy. Advise females of reproductive potential that effective contraception should be used during treatment and for 4 weeks following the final dose of upadacitinib.

### Lactation

Advise women not to breastfeed during treatment with RINVOQ® and for 6 days after the last dose.

### Administration

Advise patients not to chew, crush, or split RINVOQ® tablets.

### Medication Guide

RINVOQ® (RIN-VOKE) extended-release tablets, for oral use. What is the most important information I should know about RINVOQ®? RINVOQ® can cause serious side effects, including:

### 1. Serious Infections.

RINVOQ® is a medicine that affects your immune system. RINVOQ® can lower the ability of your immune system to fight infections. Some people have had serious infections while taking RINVOQ, including tuberculosis (TB), and infections caused by bacteria, fungi, or viruses that can spread throughout the body. Some people have died from these infections.
Your healthcare provider should test you for TB before starting treatment with RINVOQ®.
Your healthcare provider should watch you closely for signs and symptoms of TB during treatment with RINVOQ®.
You should not start taking RINVOQ® if you have any kind of infection unless your healthcare provider tells you it is okay. You may be at a higher risk of developing shingles (herpes zoster).
Before starting RINVOQ®, tell your healthcare provider if you:
   are being treated for an infection.
   have had an infection that does not go away or that keeps coming back.
   have diabetes, chronic lung disease, HIV, or a weak immune system.
   have TB or have been in close contact with someone with TB.
   have had shingles (herpes zoster).
   have or have had hepatitis B or C.
   live or have lived, or have traveled to certain parts of the country (such as the Ohio and Mississippi River valleys and the Southwest) where there is an increased chance for getting certain kinds of fungal infections. These infections may happen or become more severe if you use RINVOQ®. Ask your healthcare provider if you do not know if you have lived in an area where these infections are common.
   think you have an infection or have symptoms of an infection such as:
      fever, sweating, or chills
      muscle aches
      cough
      shortness of breath
      feeling tired
      weight loss
      warm, red, or painful skin or sores on your body
      blood in your phlegm
      diarrhea or stomach pain
      burning when you urinate or urinating more often than usual

After starting RINVOQ®, call your healthcare provider right away if you have any symptoms of an infection. RINVOQ® can make you more likely to get infections or make worse any infections that you have. If you get a serious infection, your healthcare provider may stop your treatment with RINVOQ® until your infection is controlled.

2. Increased Risk of Death in People 50 Years of Age and Older Who have at Least 1 Heart Disease (Cardiovascular) Risk Factor and are Taking a Medicine in the Class of Medicines Called Janus Kinase (JAK) Inhibitors. RINVOQ® is a JAK Inhibitor Medicine.

### 3. Cancer and Immune System Problems.

RINVOQ® may increase your risk of certain cancers by changing the way your immune system works. Lymphoma and other cancers, including skin cancers can happen in people taking RINVOQ. People taking a medicine in the class of medicines called Janus kinase (JAK) inhibitors have a higher risk of certain cancers including lymphoma and lung cancer, especially if you are a current or past smoker. Tell your healthcare provider if you have ever had any type of cancer. Follow your healthcare provider's advice about having your skin checked for skin cancer during treatment with RINVOQ®. Limit the amount of time you spend in sunlight. Avoid using tanning beds or sunlamps. Wear protective clothing when you are in the sun and use a sunscreen with a high protection factor (SPF 30 and above). This is especially important if your skin is very fair or if you have a family history of skin cancer.

4. Increased Risk of Major Cardiovascular Events Such as Heart Attack, Stroke or Death in People 50 Years of Age and Older Who have at Least 1 Heart Disease (Cardiovascular) Risk Factor and Taking a Medicine in the Class of Medicines Called JAK Inhibitors, Especially if You are a Current or Past Smoker.

Get emergency help right away if you have any symptoms of a heart attack or stroke while taking RINVOQ®, including:
discomfort in the center of your chest that lasts for more than a few minutes, or that goes away and comes back
severe tightness, pain, pressure, or heaviness in your chest, throat, neck, or jaw
pain or discomfort in your arms, back, neck, jaw, or stomach
shortness of breath with or without chest discomfort
breaking out in a cold sweat
nausea or vomiting
feeling lightheaded
weakness in one part or on one side of your body
slurred speech

### 5. Blood Clots (Thrombosis).

Blood clots in the veins of your legs (deep vein thrombosis, DVT) or lungs (pulmonary embolism, PE) and arteries (arterial thrombosis) can happen in some people taking RINVOQ®. This may be life-threatening and cause death. Blood clots in the veins of the legs (DVT) and lungs (PE) have happened more often in people who are 50 years of age and older and with at least 1 heart disease (cardiovascular) risk factor taking a medicine in the class of medicines called Janus kinase (JAK) inhibitors. Tell your healthcare provider if you have had blood clots in the veins of your legs or lungs in the past. Get medical help right away if you have signs and symptoms of blood clots during treatment with RINVOQ®, including:
swelling
sudden unexplained chest or upper back pain
pain or tenderness in one or both legs
shortness of breath or difficulty breathing

### 6. Allergic Reactions.

Symptoms such as rash (hives), trouble breathing, feeling faint or dizzy, or swelling of your lips, tongue, or throat, that may mean you are having an allergic reaction have been seen in people taking RINVOQ®. Some of these reactions were serious. If any of these symptoms occur during treatment with RINVOQ®, stop taking RINVOQ® and get emergency medical help right away.

### 7. Tears (Perforation) in the Stomach or Intestines.

Tell your healthcare provider if you have had diverticulitis (inflammation in parts of the large intestine) or ulcers in your stomach or intestines. Some people taking RINVOQ® can get tears in their stomach or intestines. This happens most often in people who take nonsteroidal anti-inflammatory drugs (NSAIDs), corticosteroids, or methotrexate. Get medical help right away if you get stomach-area pain, fever, chills, nausea, or vomiting.

### 8. Changes in Certain Laboratory Test Results.

Your healthcare provider should do blood tests before you start taking RINVOQ® and while you take RINVOQ® to check for the following:
low neutrophil and lymphocyte counts. Neutrophils and lymphocytes are types of white blood cells that help the body fight off infections.
low red blood cell counts. Red blood cells carry oxygen. Low red blood cells means you may have anemia, which may make you feel weak and tired.
increased cholesterol levels. Your healthcare provider should do blood tests to check your cholesterol levels approximately 12 weeks after you start taking RINVOQ®, and as needed.
elevated liver enzymes. Liver enzymes help to tell if your liver is functioning normally. Elevated liver enzymes may indicate that your healthcare provider needs to do additional tests on your liver.

You should not take RINVOQ® if your neutrophil count, lymphocyte count, or red blood cell count is too low or your liver tests are too high. Your healthcare provider may stop your RINVOQ treatment for a period of time if needed because of changes in these blood test results. See “What are the possible side effects of RINVOQ®?” for more information about side effects. What is RINVOQ®?

RINVOQ® is a prescription medicine that is a Janus kinase (JAK) inhibitor. RINVOQ® is used:
to treat adults with moderate to severe rheumatoid arthritis when 1 or more medicines called tumor necrosis factor (TNF) blockers have been used, and did not work well or could not be tolerated.
to treat adults with active psoriatic arthritis when 1 or more medicines called tumor necrosis factor (TNF) blockers have been used, and did not work well or could not be tolerated.
to treat adults and children 12 years of age and older with moderate to severe eczema (atopic dermatitis) that did not respond to previous treatment and their eczema is not well controlled with other pills or injections, including biologic medicines, or the use of other pills or injections is not recommended.

RINVOQ® is safe and effective in children 12 years of age and older weighing at least 88 pounds (40 kg) with atopic dermatitis. It is not known if RINVOQ® is safe and effective in children under 18 years of age with juvenile idiopathic arthritis or with psoriatic arthritis. It is not known if RINVOQ® is safe and effective in children under 12 years of age with atopic dermatitis. Do not take RINVOQ® if you are allergic to upadacitinib or any of the ingredients in RINVOQ®. See the end of this Medication Guide for a complete list of ingredients in RINVOQ®.

Before taking RINVOQ®, tell your healthcare provider about all of your medical conditions, including if you:
See “What is the most important information I should know about RINVOQ®?”
have an infection.
are a current or past smoker.
have had a heart attack, other heart problems, or stroke.
have liver problems.
have kidney problems.
have unexplained stomach (abdominal) pain, have a history of diverticulitis or ulcers in your stomach or intestines, or are taking NSAIDs.
have low red or white blood cell counts.
have recently received or are scheduled to receive an immunization (vaccine). People who take RINVOQ® should not receive live vaccines.
are pregnant or plan to become pregnant. Based on animal studies, RINVOQ® may harm your unborn baby.
   Females who are able to become pregnant:
Your healthcare provider will check whether or not you are pregnant before you start treatment with RINVOQ®.
You should use effective birth control (contraception) to avoid becoming pregnant during treatment with RINVOQ® and for 4 weeks after your last dose of RINVOQ®.
Tell your healthcare provider if you think you are pregnant or become pregnant during treatment with RINVOQ®.
If you take RINVOQ during pregnancy, contact AbbVie Inc. at 1-800-633-9110, or FDA at 1-800-FDA-1088 or www.fda.gov/medwatch to provide information about the health of you and your baby.
are breastfeeding or plan to breastfeed. RINVOQ® may pass into your breast milk. You and your healthcare provider should decide if you will take RINVOQ® or breastfeed. Do not breastfeed during treatment with RINVOQ® and for 6 days after your last dose of RINVOQ®.

Tell your healthcare provider about all the medicines you take, including prescription and over-the-counter medicines, vitamins, and herbal supplements. RINVOQ® and other medicines may affect each other causing side effects.

Especially tell your healthcare provider if you take:
medicines for fungal infections (such as ketoconazole, itraconazole, posaconazole or voriconazole) or clarithromycin (for bacterial infections) as these medicines may increase the amount of RINVOQ® in your blood.
rifampicin (for bacterial infections) or phenytoin (for neurological disorders) as these medicines may decrease the effect of RINVOQ®.
medicines that affect your immune system (such as azathioprine and cyclosporine) as these medicines may increase your risk of infection.

Ask your healthcare provider or pharmacist, if you are not sure if you are taking any of these medicines. Know the medicines you take. Keep a list of them to show your healthcare provider and pharmacist when you get a new medicine.

How should I Take RINVOQ®?
Take RINVOQ® exactly as your healthcare provider tells you to use it.
Take RINVOQ® 1 time a day with or without food.
Swallow RINVOQ® tablets whole. Do not split, crush, or chew the tablets.
If you take too much RINVOQ®, call your healthcare provider or poison control center at 1-800-222-1222, or go to the nearest hospital emergency room right away.

### What are the Possible Side Effects of RINVOQ®?

### RINVOQ May Cause Serious Side Effects, Including:

See “What is the most important information I should know about RINVOQ®?”

The most common side effects of RINVOQ® in people treated for rheumatoid arthritis and psoriatic arthritis include:
upper respiratory tract infections (common cold, sinus infections)
shingles (herpes zoster)
herpes simplex virus infections, including cold sores
bronchitis
nausea
cough
fever
acne

The most common side effects of RINVOQ® in people treated for atopic dermatitis include:
upper respiratory tract infections (common cold, sinus infections)
acne
herpes simplex virus infections, including cold sores
headache
increased blood levels of creatine phosphokinase
cough
allergic reactions
inflammation of hair follicles
nausea
stomach-area (abdominal) pain
fever
increased weight
shingles (herpes zoster)
flu
tiredness
low white blood cell count (neutropenia)
muscle pain
flu-like illness

Separation or tear to the lining of the back part of the eye (retinal detachment) has happened in people with atopic dermatitis treated with RINVOQ®. Call your healthcare provider right away if you have any sudden changes in your vision during treatment with RINVOQ®.

These are not all the possible side effects of RINVOQ®. Call your doctor for medical advice about side effects. You may report side effects to FDA at 1-800-FDA-1088.

How should I Store RINVOQ®?

Store RINVOQ® at 36° F. to 77° F. (2° C. to 25° C.).

Store RINVOQ® in the original bottle to protect it from moisture.

Keep RINVOQ® and all medicines out of the reach of children.

General Information about the Safe and Effective Use of RINVOQ®.

Medicines are sometimes prescribed for purposes other than those listed in a Medication Guide. Do not use RINVOQ® for a condition for which it was not prescribed. Do not give RINVOQ to other people, even if they have the same symptoms that you have. It may harm them. You can ask your pharmacist or healthcare provider for information about RINVOQ® that is written for health professionals.

### What are the Ingredients in RINVOQ® 15 mg Tablets?

Active ingredient: upadacitinib.
Inactive ingredients: colloidal silicon dioxide, ferrosoferric oxide, hypromellose, iron oxide red, magnesium stearate, mannitol, microcrystalline cellulose, polyvinyl alcohol, polyethylene glycol, talc, tartaric acid and titanium dioxide.

### What are the Ingredients in RINVOQ 30 mg Tablets?

Active ingredient: upadacitinib
Inactive ingredients: colloidal silicon dioxide, hypromellose, iron oxide red, magnesium stearate, mannitol, microcrystalline cellulose, polyvinyl alcohol, polyethylene glycol, talc, tartaric acid and titanium dioxide.

### Example 2. RINVOQ® Prescribing Information

### Highlights of Prescribing Information

These highlights do not include all the information needed to use RINVOQ® safely and effectively. See full prescribing information for RINVOQ®. RINVOQ® (upadacitinib) extended-release tablets, for oral use. Initial U.S. Approval: 2019.

WARNING: SERIOUS INFECTIONS, MORTALITY, MALIGNANCY, MAJOR ADVERSE CARDIOVASCULAR EVENTS (MACE), AND THROMBOSIS. See full prescribing information for complete boxed warning.

Increased risk of serious bacterial, fungal, viral, and opportunistic infections leading to hospitalization or death, including tuberculosis (TB). Interrupt treatment with RINVOQ®® if serious infection occurs until the infection is controlled. Test for latent TB before and during therapy; treat latent TB prior to use. Monitor all patients for active TB during treatment, even patients with initial negative, latent TB test.

Higher rate of all-cause mortality, including sudden cardiovascular death with another Janus kinase (JAK) inhibitor vs. tumor necrosis factor (TNF) blockers in rheumatoid arthritis (RA) patients.

Malignancies have occurred in patients treated with RINVOQ®®. Higher rate of lymphomas and lung cancers with another JAK inhibitor vs. TNF blockers in RA patients.

Higher rate of MACE (defined as cardiovascular death, myocardial infarction, and stroke) with another JAK inhibitor vs. TNF blockers in RA patients.

Thrombosis has occurred in patients treated with RINVOQ®®. Increased incidence of pulmonary embolism, venous and arterial thrombosis with another JAK inhibitor vs. TNF blockers.

### Indications and Usage

### Rheumatoid Arthritis

RINVOQ® is indicated for the treatment of adults with moderately to severely active rheumatoid arthritis who have had an inadequate response or intolerance to one or more TNF blockers.

Limitations of Use: Use of RINVOQ® in combination with other JAK inhibitors, biologic DMARDs, or with potent immunosuppressants such as azathioprine and cyclosporine is not recommended.

### Psoriatic Arthritis

RINVOQ® is indicated for the treatment of adults with active psoriatic arthritis who have had an inadequate response or intolerance to one or more TNF blockers.

Limitations of Use: Use of RINVOQ® in combination with other JAK inhibitors, biologic DMARDs, or with potent immunosuppressants such as azathioprine and cyclosporine is not recommended.

### Atopic Dermatitis

RINVOQ® is indicated for the treatment of adults and pediatric patients 12 years of age and older with refractory, moderate to severe atopic dermatitis whose disease is not adequately controlled with other systemic drug products, including biologics, or when use of those therapies are inadvisable.

Limitations of Use: RINVOQ® is not recommended for use in combination with other JAK inhibitors, biologic immunomodulators, or with other immunosuppressants.

### Ulcerative Colitis

RINVOQ® is indicated for the treatment of adult patients with moderately to severely active ulcerative colitis who have had an inadequate response or intolerance to one or more TNF blockers.

Limitations of Use: RINVOQ® is not recommended for use in combination with other JAK inhibitors, biological therapies for ulcerative colitis, or with other potent immunosuppressants such as azathioprine and cyclosporine.

### Dosage and Administration

Prior to treatment update immunizations and consider evaluating for active and latent tuberculosis, viral hepatitis, hepatic function, and pregnancy status. Avoid initiation or interrupt RINVOQ® if absolute lymphocyte count is less than 500 cells/mm³, absolute neutrophil count is less than 1000 cells/mm³, or hemoglobin level is less than 8 g/dL. RINVOQ® initiation is not recommended for patients with severe hepatic impairment (Child-Pugh C).

### Recommended Dosages

### Rheumatoid Arthritis and Psoriatic Arthritis

The recommended dosage of RINVOQ® is 15 mg once daily.

### Atopic Dermatitis

Pediatric Patients 12 Years of Age and Older Weighing at Least 40 kg and Adults Less

Than 65 Years of Age: Initiate treatment with 15 mg orally once daily. If an adequate response is not achieved, consider increasing the dosage to 30 mg orally once daily.
Adults 65 Years of Age and Older: Recommended dosage is 15 mg once daily.
Severe Renal Impairment: Recommended dosage is 15 mg once daily.

### Ulcerative Colitis

Adults: The recommended induction dosage is 45 mg once daily for 8 weeks. The recommended maintenance dosage is 15 mg once daily. A maintenance dosage of 30 mg once daily may be considered for patients with refractory, severe, or extensive disease. Discontinue RINVOQ® if adequate therapeutic response is not achieved with the 30 mg dosage. Use the lowest effective dosage needed to maintain response.
See the Full Prescribing Information for the recommended dosage in patients with renal or hepatic impairment and for dosage modification due to drug interactions.

### Dosage Forms and Strengths

Extended-release tablets: 15 mg, 30 mg, and 45 mg.

### Contraindications

Known hypersensitivity to upadacitinib or any of the excipients in RINVOQ®.

### Warnings and Precautions

Serious Infections: Avoid use of RINVOQ® in patients with active, serious infection, including localized infections.

Hypersensitivity: Serious hypersensitivity reactions (e.g., anaphylaxis) have been reported. Discontinue RINVOQ® if a serious hypersensitivity reaction occurs.

Gastrointestinal (GI) Perforations: Monitor patients at risk for GI perforations and promptly evaluate patients with symptoms.

Laboratory Abnormalities: Monitoring recommended due to potential changes in lymphocytes, neutrophils, hemoglobin, liver enzymes and lipids.

Embryo-Fetal Toxicity: RINVOQ® may cause fetal harm based on animal studies. Advise female patients of reproductive potential of the potential risk to a fetus and to use effective contraception.

Vaccinations: Avoid use of RINVOQ® with live vaccines.

### Adverse Reactions

Rheumatoid arthritis and psoriatic arthritis: Adverse reactions (>1%) were: upper respiratory tract infections, herpes zoster, herpes simplex, bronchitis, nausea, cough, pyrexia and acne.

Atopic Dermatitis: Adverse reactions (≥1%) are: upper respiratory tract infections, acne, herpes simplex, headache, blood creatine phosphokinase increased, cough, hypersensitivity, folliculitis, nausea, abdominal pain, pyrexia, increased weight, herpes zoster, influenza, fatigue, neutropenia, myalgia, and influenza like illness.

Ulcerative colitis: Adverse reactions (≥5%) reported during induction or maintenance are: upper respiratory tract infections, increased blood creatine phosphokinase, acne, neutropenia, elevated liver enzymes, and rash.

### Drug Interactions

Strong CYP3A4 Inhibitors: See the Full Prescribing Information for dosage modification for patients with atopic dermatitis and ulcerative colitis.

Strong CYP3A4 Inducers: Coadministration of RINVOQ® with strong CYP3A4 inducers is not recommended.

Use in Specific Populations

Lactation: Advise not to breastfeed.

Hepatic Impairment: RINVOQ® is not recommended in patients with severe hepatic impairment.

### Full Prescribing Information

### Warning: Serious Infections, Mortality, Malignancy, Major Adverse Cardiovascular Events, and Thrombosis

### Serious Infections

Patients treated with RINVOQ® are at increased risk for developing serious infections that may lead to hospitalization or death. Most patients who developed these infections were taking concomitant immunosuppressants such as methotrexate or corticosteroids. If a serious infection develops, interrupt RINVOQ® until the infection is controlled.

Reported infections include:
Active tuberculosis, which may present with pulmonary or extrapulmonary disease. Patients should be tested for latent tuberculosis before RINVOQ® use and during therapy. Treatment for latent infection should be considered prior to RINVOQ® use.
Invasive fungal infections, including cryptococcosis and pneumocystosis.
Bacterial, viral, including herpes zoster, and other infections due to opportunistic pathogens.

The risks and benefits of treatment with RINVOQ® should be carefully considered prior to initiating therapy in patients with chronic or recurrent infection. Patients should be closely monitored for the development of signs and symptoms of infection during and after treatment with RINVOQ®, including the possible development of tuberculosis in patients who tested negative for latent tuberculosis infection prior to initiating therapy.

### Mortality

In a large, randomized, postmarketing safety study in rheumatoid arthritis (RA) patients 50 years of age and older with at least one cardiovascular risk factor comparing another Janus kinase (JAK) inhibitor to tumor necrosis factor (TNF) blockers, a higher rate of all-cause mortality, including sudden cardiovascular death, was observed with the JAK inhibitor.

### Malignancies

Lymphoma and other malignancies have been observed in patients treated with RINVOQ®. In RA patients treated with another JAK inhibitor, a higher rate of malignancies (excluding non-melanoma skin cancer (NMSC)) was observed when compared with TNF blockers. Patients who are current or past smokers are at additional increased risk.

### Major Adverse Cardiovascular Events

In RA patients 50 years of age and older with at least one cardiovascular risk factor treated with another JAK inhibitor, a higher rate of major adverse cardiovascular events (MACE) (defined as cardiovascular death, myocardial infarction, and stroke), was observed when compared with TNF blockers. Patients who are current or past smokers are at additional increased risk. Discontinue RINVOQ® in patients that have experienced a myocardial infarction or stroke.

### Thrombosis

Thrombosis, including deep venous thrombosis, pulmonary embolism, and arterial thrombosis have occurred in patients treated with JAK inhibitors used to treat inflammatory conditions. Many of these adverse events were serious and some resulted in death. In RA patients 50 years of age and older with at least one cardiovascular risk factor treated with another JAK inhibitor, a higher rate of thrombosis was observed when compared with TNF blockers. Avoid RINVOQ® in patients at risk. Patients with symptoms of thrombosis should discontinue RINVOQ® and be promptly evaluated.

### Indications and Usage

### Rheumatoid Arthritis

RINVOQ® (upadacitinib) is indicated for the treatment of adults with moderately to severely active rheumatoid arthritis who have had an inadequate response or intolerance to one or more TNF blockers.

Limitations of Use: Use of RINVOQ® in combination with other JAK inhibitors, biologic disease-modifying antirheumatic drugs (DMARDs), or with potent immunosuppressants such as azathioprine and cyclosporine, is not recommended.

### Psoriatic Arthritis

RINVOQ® is indicated for the treatment of adults with active psoriatic arthritis who have had an inadequate response or intolerance to one or more TNF blockers.

Limitations of Use: Use of RINVOQ® in combination with other JAK inhibitors, biologic DMARDs, or with potent immunosuppressants such as azathioprine and cyclosporine, is not recommended.

### Atopic Dermatitis

RINVOQ® is indicated for the treatment of adults and pediatric patients 12 years of age and older with refractory, moderate to severe atopic dermatitis whose disease is not adequately controlled with other systemic drug products, including biologics, or when use of those therapies are inadvisable.

Limitations of Use: RINVOQ® is not recommended for use in combination with other JAK inhibitors, biologic immunomodulators, or with other immunosuppressants.

### Ulcerative Colitis

RINVOQ® is indicated for the treatment of adult patients with moderately to severely active ulcerative colitis who have had an inadequate response or intolerance to one or more TNF blockers.

Limitations of Use: RINVOQ® is not recommended for use in combination with other JAK inhibitors, biological therapies for ulcerative colitis, or with potent immunosuppressants such as azathioprine and cyclosporine.

### Dosage and Administration

### Recommended Evaluations and Immunizations Prior to Treatment Initiation

Prior to RINVOQ® treatment initiation, consider performing the following evaluations:
Active and latent tuberculosis (TB) infection evaluation—If positive, treat for TB prior to RINVOQ® use.
Viral hepatitis screening in accordance with clinical guidelines—RINVOQ® initiation is not recommended in patients with active hepatitis B or hepatitis C.
A complete blood count—RINVOQ® initiation is not recommended in patients with an absolute lymphocyte count less than 500 cells/mm3, absolute neutrophil count less than 1000 cells/mm3, or hemoglobin level less than 8 g/dL
Baseline hepatic function: RINVOQ® initiation is not recommended for patients with severe hepatic impairment (Child-Pugh C).
Pregnancy Status: Verify the pregnancy status of females of reproductive potential prior to starting treatment
Update immunizations according to current immunization guidelines

### Important Administration Instructions

RINVOQ® tablets should be taken orally with or without food

RINVOQ® tablets should be swallowed whole. RINVOQ® should not be split, crushed, or chewed.

### Recommended Dosage in Rheumatoid Arthritis

The recommended dosage of RINVOQ® is 15 mg once daily.

### Recommended Dosage in Psoriatic Arthritis

The recommended dosage of RINVOQ® is 15 mg once daily.

### Recommended Dosage in Atopic Dermatitis

Pediatric Patients 12 Years of Age and Older Weighing at Least 40 kg and Adults Less Than 65 Years of Age: Initiate treatment with 15 mg once daily. If an adequate response is not achieved, consider increasing the dosage to 30 mg once daily. Discontinue RINVOQ® if an adequate response is not achieved with the 30 mg dose. Use the lowest effective dose needed to maintain response.
Adults 65 Years of Age and Older: The recommended dosage is 15 mg once daily.

### Recommended Dosage in Ulcerative Colitis

### Adult Patients: Induction

The recommended induction dose of RINVOQ® is 45 mg once daily for 8 weeks.

### Adult Patients: Maintenance

The recommended dose of RINVOQ® for maintenance treatment is 15 mg once daily. A dosage of 30 mg once daily may be considered for patients with refractory, severe, or extensive disease. Discontinue RINVOQ® if an adequate therapeutic response is not achieved with the 30 mg dosage. Use the lowest effective dosage needed to maintain response.

Recommended Dosage in Patients with Renal Impairment or Severe Hepatic Impairment

### Renal Impairment

### Rheumatoid Arthritis and Psoriatic Arthritis:

No dosage adjustment is needed for patients with mild, moderate, or severe renal impairment.

### Atopic Dermatitis:

For patients with severe renal impairment [estimated glomerular filtration rate (eGFR) 15 to <30 mL/min/1.73 m²] the recommended dosage is 15 mg once daily.
No dosage adjustment is needed for patients with mild or moderate renal impairment (eGFR ≥30 mL/min/1.73 m²).
RINVOQ® is not recommended for use in patients with end stage renal disease (eGFR <15 mL/min/1.73 m²)

### Ulcerative Colitis:

For patients with severe renal impairment (eGFR 15 to <30 mL/min/1.73 m²), the recommended dosage is:
   Induction: 30 mg once daily for up to 8 weeks
   Maintenance: 15 mg once daily
No dosage adjustment is needed for patients with mild or moderate renal impairment (eGFR ≥30 mL/min/1.73 m²).
RINVOQ® is not recommended for use in patients with end stage renal disease (eGFR <15 mL/min/1.73 m²)

### Hepatic Impairment

RINVOQ® is not recommended for use in patients with severe hepatic impairment. Rheumatoid Arthritis, Psoriatic Arthritis, and Atopic Dermatitis:
No dosage adjustment is needed for patients with mild or moderate hepatic impairment (Child-Pugh A or B).

### Ulcerative Colitis:

For patients with mild to moderate hepatic impairment (Child-Pugh A or B) the recommended dosage is:
   Induction: 30 mg once daily for up to 8 weeks
   Maintenance: 15 mg once daily

### Dosage Modifications Due to Drug Interactions

### Rheumatoid Arthritis, Psoriatic Arthritis, Atopic Dermatitis

The recommended dosage in patients receiving strong CYP3A4 inhibitors is 15 mg once daily.

### Ulcerative Colitis

The recommended dosage in patients with ulcerative colitis receiving strong CYP3A4 inhibitors:
Induction: 30 mg once daily for up to 8 weeks
Maintenance: 15 mg once daily

### Dosage Interruption

### Infections

If a patient develops a serious infection, including serious opportunistic infection, interrupt RINVOQ® treatment until the infection is controlled.

### Laboratory Abnormalities

Interruption of dosing may be needed for management of laboratory abnormalities as described in Table 17.

TABLE 17
Recommended Dosage Interruptions for Laboratory Abnormalities

| Laboratory Measure | Action |
|---|---|
| Absolute Neutrophil Count (ANC) | Interrupt treatment if ANC is less than 1000 cells/mm³; |
| | treatment may be restarted once ANC returns above this value |
| Absolute Lymphocyte Count (ALC) | Interrupt treatment if ALC is less than 500 cells/mm³; |
| | treatment may be restarted once ALC returns above this value |
| Hemoglobin (Hb) | Interrupt treatment if Hb is less than 8 g/dL; treatment |
| | may be restarted once Hb returns above this value |
| Hepatic transaminases | Interrupt treatment if drug-induced liver injury is |
| | suspected, until this diagnosis is excluded. |

### Dosage Forms and Strengths

Extended-Release Tablets:
15 mg: purple, biconvex oblong, with dimensions of 14×8 mm, and debossed with ‘a15’ on one side.
30 mg: red, biconvex oblong, with dimensions of 14×8 mm, and debossed with ‘a30’ on one side.
45 mg: yellow to mottled yellow, biconvex oblong, with dimensions of 14×8 mm, and debossed with ‘a45’ on one side.

### Contraindications

RINVOQ® is contraindicated in patients with known hypersensitivity to upadacitinib or any of its excipients.

### Warnings and Precautions

### Serious Infections

Serious and sometimes fatal infections have been reported in patients receiving RINVOQ®. The most frequent serious infections reported with RINVOQ® included pneumonia and cellulitis. Among opportunistic infections, tuberculosis, multidermatomal herpes zoster, oral/esophageal candidiasis, and cryptococcosis, were reported with RINVOQ®. Avoid use of RINVOQ® in patients with an active, serious infection, including localized infections.

Consider the risks and benefits of treatment prior to initiating RINVOQ® in patients:
with chronic or recurrent infection
who have been exposed to tuberculosis
with a history of a serious or an opportunistic infection
who have resided or traveled in areas of endemic tuberculosis or endemic mycoses; or
with underlying conditions that may predispose them to infection.

Closely monitor patients for the development of signs and symptoms of infection during and after treatment with RINVOQ®. Interrupt RINVOQ® if a patient develops a serious or opportunistic infection. A patient who develops a new infection during treatment with RINVOQ® should undergo prompt and complete diagnostic testing appropriate for an immunocompromised patient; appropriate antimicrobial therapy should be initiated, the patient should be closely monitored, and RINVOQ® should be interrupted if the patient is not responding to antimicrobial therapy. RINVOQ® may be resumed once the infection is controlled.

### Tuberculosis

Evaluate and test patients for latent and active tuberculosis (TB) infection prior to administration of RINVOQ®. Patients with latent TB should be treated with standard antimycobacterial therapy before initiating RINVOQ®. RINVOQ® should not be given to patients with active TB. Consider anti-TB therapy prior to initiation of RINVOQ® in patients with previously untreated latent TB or active TB in whom an adequate course of treatment cannot be confirmed, and for patients with a negative test for latent TB but who have risk factors for TB infection. Consultation with a physician with expertise in the treatment of TB is recommended to aid in the decision about whether initiating anti-TB therapy is appropriate for an individual patient. During RINVOQ® use, monitor patients for the development of signs and symptoms of TB, including patients who tested negative for latent TB infection prior to initiating therapy.

### Viral Reactivation

Viral reactivation, including cases of herpes virus reactivation (e.g., herpes zoster) and hepatitis B virus reactivation, were reported in clinical trials with RINVOQ®. The risk of herpes zoster appears to be higher in patients treated with RINVOQ® in Japan. If a patient develops herpes zoster, consider temporarily interrupting RINVOQ® until the episode resolves. Screening for viral hepatitis and monitoring for reactivation should be performed in accordance with clinical guidelines before starting and during therapy with RINVOQ®. Patients who were positive for hepatitis C antibody and hepatitis C virus RNA, were excluded from clinical trials. Patients who were positive for hepatitis B surface antigen or hepatitis B virus DNA were excluded from clinical trials. However, cases of hepatitis B reactivation were still reported in patients enrolled in the Phase 3 trials of RINVOQ®. If hepatitis B virus DNA is detected while receiving RINVOQ®, a liver specialist should be consulted.

### Mortality

In a large, randomized, post-marketing safety study of another JAK inhibitor in RA patients 50 years of age and older with at least one cardiovascular risk factor, a higher rate of all-cause mortality, including sudden cardiovascular death, was observed in patients treated with the JAK inhibitor compared with TNF blockers. Consider the benefits and risks for the individual patient prior to initiating or continuing therapy with RINVOQ®.

### Malignancy and Lymphoproliferative Disorders

Malignancies, including lymphomas, were observed in clinical trials of RINVOQ®. In a large, randomized, post-marketing safety study of another JAK inhibitor in RA patients, a higher rate of malignancies (excluding non-melanoma skin cancer (NMSC)) was observed in patients treated with the JAK inhibitor compared to those treated with TNF blockers. A higher rate of lymphomas was observed in patients treated with the JAK inhibitor compared to those treated with TNF blockers. A higher rate of lung cancers was observed in current or past smokers treated with the JAK inhibitor compared to those treated with TNF blockers. In this study, current or past smokers had an additional increased risk of overall malignancies. Consider the benefits and risks for the individual patient prior to initiating or continuing therapy with RINVOQ®, particularly in patients with a known malignancy (other than a successfully treated NMSC), patients who develop a malignancy when on treatment, and patients who are current or past smokers.

### Non-Melanoma Skin Cancer

NMSCs have been reported in patients treated with RINVOQ®. Periodic skin examination is recommended for patients who are at increased risk for skin cancer. Exposure to sunlight and UV light should be limited by wearing protective clothing and using a broad-spectrum sunscreen.

### Major Adverse Cardiovascular Events

In a large, randomized, post marketing safety study of another JAK inhibitor in RA patients 50 years of age and older with at least one cardiovascular risk factor, a higher rate of major adverse cardiovascular events (MACE) defined as cardiovascular death, non-fatal myocardial infarction (MI), and non-fatal stroke was observed with the JAK inhibitor compared to those treated with TNF blockers. Patients who are current or past smokers are at additional increased risk.

Consider the benefits and risks for the individual patient prior to initiating or continuing therapy with RINVOQ®, particularly in patients who are current or past smokers and patients with other cardiovascular risk factors. Patients should be informed about the symptoms of serious cardiovascular events and the steps to take if they occur. Discontinue RINVOQ® in patients that have experienced a myocardial infarction or stroke.

### Thrombosis

Thrombosis, including deep venous thrombosis (DVT), pulmonary embolism (PE), and arterial thrombosis, have occurred in patients treated for inflammatory conditions with JAK inhibitors, including RINVOQ®. Many of these adverse events were serious and some resulted in death.

In a large, randomized, post-marketing safety study of another JAK inhibitor in RA patients 50 years of age and older with at least one cardiovascular risk factor, higher rates of overall thrombosis, DVT, and PE were observed compared to those treated with TNF blockers. If symptoms of thrombosis occur, patients should discontinue RINVOQ® and be evaluated promptly and treated appropriately. Avoid RINVOQ® in patients that may be at increased risk of thrombosis.

### Hypersensitivity Reactions

Serious hypersensitivity reactions such as anaphylaxis and angioedema were reported in patients receiving RINVOQ® in clinical trials. If a clinically significant hypersensitivity reaction occurs, discontinue RINVOQ® and institute appropriate therapy.

### Gastrointestinal Perforations

Gastrointestinal perforations have been reported in clinical trials with RINVOQ®. In these trials, many patients with rheumatoid arthritis were receiving background therapy with nonsteroidal anti-inflammatory drugs (NSAIDs).

Monitor RINVOQ®-treated patients who may be at risk for gastrointestinal perforation (e.g., patients with a history of diverticulitis or taking NSAIDs). Evaluate promptly patients presenting with new onset abdominal pain for early identification of gastrointestinal perforation.

### Laboratory Abnormalities

### Neutropenia

Treatment with RINVOQ® was associated with an increased incidence of neutropenia (ANC less than 1000 cells/mm³). Evaluate neutrophil counts at baseline and thereafter according to routine patient management. Avoid RINVOQ® initiation and interrupt RINVOQ® treatment in patients with a low neutrophil count (i.e., ANC less than 1000 cells/mm³).

### Lymphopenia

ALC less than 500 cells/mm³ were reported in RINVOQ®-treated patients in clinical trials. Evaluate lymphocyte counts at baseline and thereafter according to routine patient management. Avoid RINVOQ® initiation or interrupt RINVOQ® treatment in patients with a low lymphocyte count (i.e., less than 500 cells/mm³).

### Anemia

Decreases in hemoglobin levels to less than 8 g/dL were reported in RINVOQ®-treated patients in clinical trials. Evaluate hemoglobin at baseline and thereafter according to routine patient management. Avoid RINVOQ® initiation or interrupt RINVOQ® treatment in patients with a low hemoglobin level (i.e., less than 8 g/dL).

### Lipids

Treatment with RINVOQ® was associated with increases in lipid parameters, including total cholesterol, low-density lipoprotein (LDL) cholesterol, and high-density lipoprotein (HDL) cholesterol. Elevations in LDL cholesterol decreased to pre-treatment levels in response to statin therapy. The effect of these lipid parameter elevations on cardiovascular morbidity and mortality has not been determined. Assess lipid parameters approximately 12 weeks after initiation of treatment, and thereafter according to the clinical guidelines for hyperlipidemia. Manage patients according to clinical guidelines for the management of hyperlipidemia.

### Liver Enzyme Elevations

Treatment with RINVOQ® was associated with increased incidence of liver enzyme elevations compared to treatment with placebo. Evaluate liver enzymes at baseline and thereafter according to routine patient management. Prompt investigation of the cause of liver enzyme elevation is recommended to identify potential cases of drug-induced liver injury. If increases in ALT or AST are observed during routine patient management and drug-induced liver injury is suspected, RINVOQ® should be interrupted until this diagnosis is excluded.

### Embryo-Fetal Toxicity

Based on findings in animal studies, RINVOQ® may cause fetal harm when administered to a pregnant woman. Administration of upadacitinib to rats and rabbits during organogenesis caused increases in fetal malformations. Verify the pregnancy status of patients of reproductive potential prior to starting treatment. Advise females of reproductive potential of the potential risk to the fetus and to use effective contraception during treatment with RINVOQ® and for 4 weeks following completion of therapy.

### Vaccinations

Avoid use of live vaccines during, or immediately prior to, RINVOQ® therapy. Prior to initiating RINVOQ®, it is recommended that patients be brought up to date with all immunizations, including varicella zoster or prophylactic herpes zoster vaccinations, in agreement with current immunization guidelines.

### Adverse Reactions

The following clinically significant adverse reactions are described elsewhere in the labeling:
Serious Infections
Mortality
Malignancy and Lymphoproliferative Disorders
Major Adverse Cardiovascular Events
Thrombosis
Hypersensitivity Reactions
Gastrointestinal Perforations
Laboratory Abnormalities

### Clinical Trials Experience

Because clinical trials are conducted under widely varying conditions, adverse reaction rates observed in the clinical trials of a drug cannot be directly compared to rates in the clinical trials of another drug and may not reflect the rates observed in practice.

Adverse Reactions in Patients with Rheumatoid Arthritis

A total of 3833 patients with rheumatoid arthritis were treated with upadacitinib in the Phase 3 clinical trials of whom 2806 were exposed for at least one year. Patients could advance or switch to RINVOQ® 15 mg from placebo, or be rescued to RINVOQ® from active comparator or placebo from as early as Week 12 depending on the trial design. A total of 2630 patients received at least 1 dose of RINVOQ® 15 mg, of whom 1860 were exposed for at least one year. In trials RA-I, RA-II, RA-III and RA-V, 1213 patients received at least 1 dose of RINVOQ® 15 mg, of which 986 patients were exposed for at least one year, and 1203 patients received at least 1 dose of upadacitinib 30 mg, of which 946 were exposed for at least one year.

TABLE 18
Adverse Reactions Reported in ≥1% of Rheumatoid
Arthritis Patients Treated with RINVOQ ® 15
mg in Placebo-controlled Trials

| | Placebo | RINVOQ ® 15 mg |
|---|---|---|
| | n = 1042 | n = 1035 |
| Adverse Reaction | (%) | (%) |
| Upper respiratory tract | 9.5 | 13.5 |
| infection (URTI)* | | |
| Nausea | 2.2 | 3.5 |
| Cough | 1.0 | 2.2 |
| Pyrexia | 0 | 1.2 |

*URTI includes: acute sinusitis, laryngitis, nasopharyngitis, oropharyngeal pain, pharyngitis, pharyngotonsillitis, rhinitis, sinusitis, tonsillitis, viral upper respiratory tract infection

Other adverse reactions reported in less than 1% of patients in the RINVOQ® 15 mg group and at a higher rate than in the placebo group through Week 12 included pneumonia, herpes zoster, herpes simplex (includes oral herpes), and oral candidiasis. Four integrated datasets are presented in the Specific Adverse Reaction section:

Placebo-controlled Trials: Trials RA-III, RA-IV, and RA-V were integrated to represent safety through 12/14 weeks for placebo (n=1042) and RINVOQ® 15 mg (n=1035). Trials RA-III and RA-V were integrated to represent safety through 12 weeks for placebo (n=390), RINVOQ® 15 mg (n=385), and upadacitinib 30 mg (n=384). Trial RA-IV did not include the 30 mg dose and, therefore, safety data for upadacitinib 30 mg can only be compared with placebo and RINVOQ® 15 mg rates from pooling trials RA-III and RA-V. MTX-controlled Trials: Trials RA-I and RA-II were integrated to represent safety through 12/14 weeks for MTX (n=530), RINVOQ® 15 mg (n=534), and upadacitinib 30 mg (n=529). 12-Month Exposure Dataset: Trials RA-I, II, III, and V were integrated to represent the long-term safety of RINVOQ® 15 mg (n=1213) and upadacitinib 30 mg (n=1203). Exposure adjusted incidence rates were adjusted by trial for all the adverse events reported in this section.

### Specific Adverse Reactions

### Infections

Placebo-controlled Trials: In RA-III, RA-IV, and RA-V, infections were reported in 218 patients (95.7 per 100 patient-years) treated with placebo and 284 patients (127.8 per 100 patient-years) treated with RINVOQ® 15 mg. In RA-III and RA-V, infections were reported in 99 patients (136.5 per 100 patient-years) treated with placebo, 118 patients (164.5 per 100 patient-years) treated with RINVOQ® 15 mg, and 126 patients (180.3 per 100 patient-years) treated with upadacitinib 30 mg.

MTX-controlled Trials: Infections were reported in 127 patients (119.5 per 100 patient-years) treated with MTX monotherapy, 104 patients (91.8 per 100 patient-years) treated with RINVOQ® 15 mg monotherapy, and 128 patients (115.1 per 100 patient-years) treated with upadacitinib 30 mg monotherapy.

12-Month Exposure Dataset: Infections were reported in 615 patients (83.8 per 100 patient-years) treated with RINVOQ® 15 mg and 674 patients (99.7 per 100 patient-years) treated with upadacitinib 30 mg.

### Serious Infections

Placebo-controlled Trials: In RA-III, RA-IV, and RA-V, serious infections were reported in 6 patients (2.3 per 100 patient-years) treated with placebo, and 12 patients (4.6 per 100 patient-years) treated with RINVOQ® 15 mg. In RA-III and RA-V, serious infections were reported in 1 patient (1.2 per 100 patient-years) treated with placebo, 2 patients (2.3 per 100 patient-years) treated with RINVOQ® 15 mg, and 7 patients (8.2 per 100 patient-years) treated with upadacitinib 30 mg.

MTX-controlled Trials: Serious infections were reported in 2 patients (1.6 per 100 patient-years) treated with MTX monotherapy, 3 patients (2.4 per 100 patient-years) treated with RINVOQ® 15 mg monotherapy, and 8 patients (6.4 per 100 patient-years) treated with upadacitinib 30 mg monotherapy.

12-Month Exposure Dataset: Serious infections were reported in 38 patients (3.5 per 100 patient-years) treated with RINVOQ® 15 mg and 59 patients (5.6 per 100 patient-years) treated with upadacitinib 30 mg.

The most frequently reported serious infections were pneumonia and cellulitis.

### Tuberculosis

Placebo-controlled Trials and MTX-controlled Trials: In the placebo-controlled period, there were no active cases of tuberculosis reported in the placebo, RINVOQ® 15 mg, and upadacitinib 30 mg groups. In the MTX-controlled period, there were no active cases of tuberculosis reported in the MTX monotherapy, RINVOQ® 15 mg monotherapy, and upadacitinib 30 mg monotherapy groups.

12-Month Exposure Dataset: Active tuberculosis was reported for 2 patients treated with RINVOQ® 15 mg and 1 patient treated with upadacitinib 30 mg. Cases of extra-pulmonary tuberculosis were reported.

### Opportunistic Infections (Excluding Tuberculosis)

Placebo-controlled Trials: In RA-III, RA-IV, and RA-V, opportunistic infections were reported in 3 patients (1.2 per 100 patient-years) treated with placebo, and 5 patients (1.9 per 100 patient-years) treated with RINVOQ® 15 mg. In RA-III and RA-V, opportunistic infections were reported in 1 patient (1.2 per 100 patient-years) treated with placebo, 2 patients (2.3 per 100 patient-years) treated with RINVOQ® 15 mg, and 6 patients (7.1 per 100 patient-years) treated with upadacitinib 30 mg.

MTX-controlled Trials: Opportunistic infections were reported in 1 patient (0.8 per 100 patient-years) treated with MTX monotherapy, 0 patients treated with RINVOQ® 15 mg monotherapy, and 4 patients (3.2 per 100 patient-years) treated with upadacitinib 30 mg monotherapy.

12-Month Exposure Dataset: Opportunistic infections were reported in 7 patients (0.6 per 100 patient-years) treated with RINVOQ® 15 mg and 15 patients (1.4 per 100 patient-years) treated with upadacitinib 30 mg.

### Malignancies

Placebo-controlled Trials: In RA-III, RA-IV, and RA-V, malignancies excluding NMSC were reported in 1 patient (0.4 per 100 patient-years) treated with placebo, and 1 patient (0.4 per 100 patient-years) treated with RINVOQ® 15 mg. In RA-III and RA-V, malignancies excluding NMSC were reported in 0 patients treated with placebo, 1 patient (1.1 per 100 patient-years) treated with RINVOQ® 15 mg, and 3 patients (3.5 per 100 patient-years) treated with upadacitinib 30 mg.

MTX-controlled Trials: Malignancies excluding NMSC were reported in 1 patient (0.8 per 100 patient-years) treated with MTX monotherapy, 3 patients (2.4 per 100 patient-years) treated with RINVOQ® 15 mg monotherapy, and 0 patients treated with upadacitinib 30 mg monotherapy.

12-Month Exposure Dataset: Malignancies excluding NMSC were reported in 13 patients (1.2 per 100 patient-years) treated with RINVOQ® 15 mg and 14 patients (1.3 per 100 patient-years) treated with upadacitinib 30 mg.

### Gastrointestinal Perforations

Placebo-controlled Trials: There were no gastrointestinal perforations (based on medical review) reported in patients treated with placebo, RINVOQ® 15 mg, and upadacitinib 30 mg.

MTX-controlled Trials: There were no cases of gastrointestinal perforations reported in the MTX and RINVOQ® 15 mg group through 12/14 weeks. Two cases of gastrointestinal perforations were observed in the upadacitinib 30 mg group.

12-Month Exposure Dataset: Gastrointestinal perforations were reported in 1 patient treated with RINVOQ® 15 mg and 4 patients treated with upadacitinib 30 mg.

### Thrombosis

Placebo-controlled Trials: In RA-IV, venous thrombosis (pulmonary embolism or deep vein thrombosis) was observed in 1 patient treated with placebo and 1 patient treated with RINVOQ® 15 mg. In RA-V, venous thrombosis was observed in 1 patient treated with RINVOQ® 15 mg. There were no observed cases of venous thrombosis reported in RA-III. No cases of arterial thrombosis were observed through 12/14 weeks.

MTX-controlled Trials: In RA-II, venous thrombosis was observed in 0 patients treated with MTX monotherapy, 1 patient treated with RINVOQ® 15 mg monotherapy and 0 patients treated with upadacitinib 30 mg monotherapy through Week 14. In RA-II, no cases of arterial thrombosis were observed through 12/14 weeks. In RA-I, venous thrombosis was observed in 1 patient treated with MTX, 0 patients treated with RINVOQ® 15 mg and 1 patient treated with upadacitinib 30 mg through Week 24. In RA-I, arterial thrombosis was observed in 1 patient treated with upadacitinib 30 mg through Week 24.

12-Month Exposure Dataset: Venous thrombosis events were reported in 5 patients (0.5 per 100 patient-years) treated with RINVOQ® 15 mg and 4 patients (0.4 per 100 patient-years) treated with upadacitinib 30 mg. Arterial thrombosis events were reported in 0 patients treated with RINVOQ® 15 mg and 2 patients (0.2 per 100 patient-years) treated with upadacitinib 30 mg.

### Laboratory Abnormalities

### Hepatic Transaminase Elevations

In placebo-controlled trials (RA-III, RA-IV, and RA-V) with background DMARDs, for up to 12/14 weeks, alanine transaminase (ALT) and aspartate transaminase (AST) elevations ≥3×upper limit of normal (ULN) in at least one measurement were observed in 2.1% and 1.5% of patients treated with RINVOQ® 15 mg, and in 1.5% and 0.7% of patients treated with placebo, respectively. In RA-III and RA-V, ALT and AST elevations >3×ULN in at least one measurement were observed in 0.8% and 1.0% of patients treated with RINVOQ® 15 mg, 1.0% and 0% of patients treated with upadacitinib 30 mg and in 1.3% and 1.0% of patients treated with placebo, respectively.

In MTX-controlled trials, for up to 12/14 weeks, ALT and AST elevations >3×ULN in at least one measurement were observed in 0.8% and 0.4% of patients treated with RINVOQ® 15 mg, 1.7% and 1.3% of patients treated with upadacitinib 30 mg and in 1.9% and 0.9% of patients treated with MTX, respectively.

### Lipid Elevations

Upadacitinib treatment was associated with dose-related increases in total cholesterol, triglycerides and LDL cholesterol. Upadacitinib was also associated with increases in HDL cholesterol. Elevations in LDL and HDL cholesterol peaked by Week 8 and remained stable thereafter. In controlled trials, for up to 12/14 weeks, changes from baseline in lipid parameters in patients treated with RINVOQ® 15 mg and upadacitinib 30 mg, respectively, are summarized below:
Mean LDL cholesterol increased by 14.81 mg/dL and 17.17 mg/dL.
Mean HDL cholesterol increased by 8.16 mg/dL and 9.01 mg/dL.
The mean LDL/HDL ratio remained stable.
Mean triglycerides increased by 13.55 mg/dL and 14.44 mg/dL.

In placebo-controlled trials (RA-III, RA-IV, and RA-V) with background DMARDs, for up to 12/14 weeks, dose-related increases in creatine phosphokinase (CPK) values were observed. CPK elevations >5×ULN were reported in 1.0%, and 0.3% of patients over 12/14 weeks in the RINVOQ® 15 mg and placebo groups, respectively. Most elevations >5×ULN were transient and did not require treatment discontinuation. In RA-III and RA-V, CPK elevations >5×ULN were observed in 0.3% of patients treated with placebo, 1.6% of patients treated with RINVOQ® 15 mg, and none in patients treated with upadacitinib 30 mg.

### Neutropenia

In placebo-controlled trials (RA-III, RA-IV, and RA-V) with background DMARDs, for up to 12/14 weeks, dose-related decreases in neutrophil counts, below 1000 cells/mm³ in at least one measurement occurred in 1.1% and <0.1% of patients in the RINVOQ® 15 mg and placebo groups, respectively. In RA-III and RA-V, decreases in neutrophil counts below 1000 cells/mm³ in at least one measurement occurred in 0.3% of patients treated with placebo, 1.3% of patients treated with RINVOQ® 15 mg, and 2.4% of patients treated with upadacitinib 30 mg. In clinical trials, treatment was interrupted in response to ANC less than 1000 cells/mm³.

### Lymphopenia

In placebo-controlled trials (RA-III, RA-IV, and RA-V) with background DMARDs, for up to 12/14 weeks, dose-related decreases in lymphocyte counts below 500 cells/mm³ in at least one measurement occurred in 0.9% and 0.7% of patients in the RINVOQ® 15 mg and placebo groups, respectively. In RA-III and RA-V, decreases in lymphocyte counts below 500 cells/mm³ in at least one measurement occurred in 0.5% of patients treated with placebo, 0.5% of patients treated with RINVOQ® 15 mg, and 2.4% of patients treated with upadacitinib 30 mg.

### Anemia

In placebo-controlled trials (RA-III, RA-IV, and RA-V) with background DMARDs, for up to 12/14 weeks, hemoglobin decreases below 8 g/dL in at least one measurement occurred in <0.1% of patients in both the RINVOQ® 15 mg and placebo groups. In RA-III and RA-V, hemoglobin decreases below 8 g/dL in at least one measurement were observed in 0.3% of patients treated with placebo, and none in patients treated with RINVOQ® 15 mg and upadacitinib 30 mg.

Adverse Reactions in Patients with Psoriatic Arthritis

A total of 1827 patients with psoriatic arthritis were treated with upadacitinib in clinical studies representing 1639.2 patient-years of exposure, of whom 722 were exposed to upadacitinib for at least one year. In the two Phase 3 studies, 907 patients received at least 1 dose of RINVOQ® 15 mg, of whom 359 were exposed for at least one year. Two placebo-controlled studies were integrated (640 patients on RINVOQ® 15 mg once daily and 635 patients on placebo) to evaluate the safety of RINVOQ® 15 mg in comparison to placebo for up to 24 weeks after treatment initiation. Overall, the safety profile observed in patients with active psoriatic arthritis treated with RINVOQ® 15 mg was consistent with the safety profile observed in patients with rheumatoid arthritis. During the 24-week placebo-controlled period, the frequencies of herpes zoster and herpes simplex were >1% (1.1% and 1.4%, respectively) with RINVOQ® 15 mg and 0.8% and 1.3%, respectively with placebo. A higher incidence of acne and bronchitis was also observed in patients treated with RINVOQ® 15 mg (1.3% and 3.9%, respectively) compared to placebo (0.3% and 2.7%, respectively).

Adverse Reactions in Patients with Atopic Dermatitis

Three Phase 3 (AD-1, AD-2, and AD-3) and one Phase 2b (AD-4) randomized, double-blind, placebo-controlled, multicenter trials evaluated the safety of RINVOQ® in patients with moderate-to-severe atopic dermatitis. The majority of patients were White (68%) and male (57%). The mean age was 34 years (ranged from 12 to 75 years) and 13% of the patients were 12 to less than 18 years. In these 4 trials, 2612 patients were treated with RINVOQ® 15 mg or 30 mg orally once daily, with or without concomitant topical corticosteroids (TCS). In the Phase 3 clinical trials (AD-1, AD-2, and AD-3), a total of 1239 patients received RINVOQ® 15 mg, of whom 791 were exposed for at least one year and 1246 patients received RINVOQ® 30 mg, of whom 826 were exposed for at least one year. Trials AD-1, AD-2, and AD-4 compared the safety of RINVOQ® monotherapy to placebo through Week 16. Trial AD-3 compared the safety of RINVOQ®+TCS to placebo+TCS through Week 16.

### Weeks 0 to 16 (Trials AD-1 to AD-4)

In RINVOQ® trials with and without TCS (Trials AD-1, 2, 3 and 4) through Week 16, the proportion of patients who discontinued treatment because of adverse reactions in the RINVOQ® 15 mg, 30 mg and placebo groups were 2.3%, 2.9% and 3.8%, respectively. Table 19 summarizes the adverse reactions that occurred at a rate of at least 1% in the RINVOQ® 15 mg or 30 mg groups during the first 16 weeks of treatment.

TABLE 19
Adverse Reactions Reported in ≥1% of Patients with Atopic Dermatitis
Treated with RINVOQ ® 15 mg or 30 mg

| | | RINVOQ ® | RINVOQ ® |
|---|---|---|---|
| | Placebo | 15 mg | 30 mg |
| | n = 902 | n = 899 | n = 906 |
| Adverse Reaction | (%) | (%) | (%) |
| Upper respiratory tract | 17 | 23 | 25 |
| infection (URTI)* | | | |
| Acne** | 2 | 10 | 16 |
| Herpes simplex*** | 2 | 4 | 8 |
| Headache | 4 | 6 | 6 |
| Increased blood creatine | 2 | 5 | 6 |
| phosphokinase | | | |
| Cough | 1 | 3 | 3 |
| Hypersensitivity**** | 2 | 2 | 3 |
| Folliculitis | 1 | 2 | 3 |
| Nausea | 1 | 3 | 3 |
| Abdominal pain***** | 1 | 3 | 2 |
| Pyrexia | 1 | 2 | 2 |
| Increased Weight | 1 | 2 | 2 |
| Herpes zoster****** | 1 | 2 | 2 |
| Influenza | <1 | 2 | 2 |
| Fatigue | 1 | 1 | 2 |
| Neutropenia | <1 | 1 | 2 |
| Myalgia | 1 | 1 | 2 |
| Influenza like illness | 1 | 1 | 2 |

*Includes: laryngitis, laryngitis viral, nasopharyngitis, oropharyngeal pain, pharyngeal abscess, pharyngitis, pharyngitis streptococcal, pharyngotonsillitis, respiratory tract infection, respiratory tract infection viral, rhinitis, rhinolaryngitis, sinusitis, tonsillitis, tonsillitis bacterial, upper respiratory tract infection, viral pharyngitis, viral upper respiratory tract infection
**Includes: acne and dermatitis acneiform
***Includes: genital herpes, genital herpes simplex, herpes dermatitis, herpes ophthalmic, herpes simplex, nasal herpes, ophthalmic herpes simplex, herpes virus infection, oral herpes
****Includes anaphylactic reaction, anaphylactic shock, angioedema, dermatitis exfoliative generalized, drug hypersensitivity, eyelid oedema, face oedema, hypersensitivity, periorbital swelling, pharyngeal swelling, swelling face, toxic skin eruption, type I hypersensitivity, urticaria
*****Includes abdominal pain and abdominal pain upper
******Includes herpes zoster and varicella

Other adverse reactions reported in less than 1% of patients in the RINVOQ® 15 mg and/or 30 mg group and at a higher rate than in the placebo group through Week 16 included anemia, oral candidiasis, pneumonia, and the adverse event of retinal detachment. The safety profile of RINVOQ® through Week 52 was generally consistent with the safety profile observed at Week 16. Overall, the safety profile observed in patients with AD treated with RINVOQ® was similar to the safety profile in patients with RA. Other specific adverse reactions that were reported in patients with AD included eczema herpeticum/Kaposi's varicelliform eruption.

### Eczema Herpeticum/Kaposi's Varicelliform Eruption

Placebo-controlled Period (16 weeks): Eczema herpeticum was reported in 4 patients (1.6 per 100 patient-years) treated with placebo, 6 patients (2.2 per 100 patient-years) treated with RINVOQ® 15 mg and 7 patients (2.6 per 100 patient-years) treated with RINVOQ® 30 mg.

12-Month Exposure (Weeks 0 to 52): Eczema herpeticum was reported in 18 patients (1.6 per 100 patient-years) treated with RINVOQ® 15 mg and 17 patients (1.5 per 100 patient-years) treated with RINVOQ® 30 mg.

Adverse Reactions in Patients with Ulcerative Colitis

RINVOQ® was studied up to 8 weeks in patients with moderately to severely active ulcerative colitis in two randomized, double-blind, placebo-controlled induction studies (UC-1, UC-2) and a randomized, double-blind, placebo controlled, dose-finding study (UC-4; NCT02819635). Long term safety up to 52-weeks was evaluated in patients who responded to induction therapy in a randomized, double-blind, placebo-controlled maintenance study (UC-3) and a long-term extension study.

In the two induction studies (UC-1, UC-2) and a dose finding study (UC-4), 109 patients were enrolled of whom 719 patients received RINVOQ® 45 mg, once daily.

In the maintenance study (UC-3), 746 patients were enrolled of whom 250 patients received RINVOQ® 15 mg once daily and 251 patients received RINVOQ® 30 mg once daily.

Adverse reactions reported in ≥2% of patients in any treatment arm in the induction and maintenance studies are shown in Tables 20 and 21, respectively.

TABLE 20
Adverse Reactions Reported in ≥2% of Patients with
Ulcerative Colitis Treated with RINVOQ ® 45
mg in Placebo-Controlled Induction Studies (UC-1, UC-2 and UC-4)

| | | RINVOQ ® 45 mg |
|---|---|---|
| | Placebo | Once Daily |
| | N = 378 | N = 719 |
| Adverse Reaction | (%) | (%) |
| Upper respiratory tract | 7 | 9 |
| infection* | | |
| Acne* | 1 | 6 |
| Increased blood creatine | 1 | 5 |
| phosphokinase | | |
| Neutropenia* | <1 | 5 |
| Rash* | 1 | 4 |
| Elevated liver enzymes** | 2 | 3 |
| Lymphopenia* | 1 | 3 |
| Folliculitis | 1 | 2 |
| Herpes simplex* | <1 | 2 |

*Composed of several similar terms
**Elevated liver enzymes composed of elevated ALT, AST, GGT, ALP, liver transaminases, hepatic enzymes, bilirubin, drug-induced liver injury and cholestasis.

TABLE 21
Adverse Reactions Reported in ≥2% of Patients with
Ulcerative Colitis Treated with RINVOQ ® 15
mg or 30 mg in the Placebo-Controlled Maintenance Study (UC-3)¹

| | | RINVOQ ® | RINVOQ ® |
|---|---|---|---|
| | | 15 mg | 30 mg |
| | Placebo | Once Daily | Once Daily |
| | n = 245 | n = 250 | n = 251 |
| Adverse Reaction | (%) | (%) | (%) |
| Upper respiratory tract | 18 | 16 | 20 |
| infection* | | | |
| Increased blood creatine | 2 | 6 | 8 |
| phosphokinase | | | |
| Neutropenia* | 2 | 3 | 6 |
| Elevated liver enzymes** | 1 | 6 | 4 |
| Rash* | 4 | 5 | 5 |
| Herpes zoster | 0 | 4 | 4 |
| Folliculitis | 2 | 2 | 4 |
| Hypercholesterolemia* | 1 | 2 | 4 |
| Influenza | 1 | 3 | 3 |
| Herpes simplex* | 1 | 2 | 3 |
| Lymphopenia* | 2 | 3 | 2 |
| Hyperlipidemia* | 0 | 2 | 2 |

¹Patients who were responders to 8 weeks induction therapy with RINVOQ ® 45 mg once daily
*Composed of several similar terms
**Elevated liver enzymes composed of elevated ALT, AST, GGT, ALP, liver transaminases, hepatic enzymes, bilirubin, drug-induced liver injury, and cholestasis.

The safety profile of RINVOQ® in the long term extension study was similar to the safety profile observed in the placebo-controlled induction and maintenance periods.

Overall, the safety profile observed in patients with ulcerative colitis treated with RINVOQ® was generally similar to the safety profile in patients with RA and AD.

### Specific Adverse Reactions

### Serious Infections

Induction Studies: In UC-1, UC-2, and UC-4, serious infections were reported in 5 patients (8.4 per 100 patient-years) treated with placebo and 9 patients (8.4 per 100 patient-years) treated with RINVOQ® 45 mg through 8 weeks.

Placebo-controlled Maintenance Study: In UC-3, serious infections were reported in 8 patients (6.3 per 100 patient-years) treated with placebo, 8 patients (4.5 per 100 patient-years) treated with RINVOQ® 15 mg, and 6 patients (3.1 per 100 patient-years) treated with RINVOQ® 30 mg through 52 weeks.

### Laboratory Abnormalities

### Hepatic Transaminase Elevations

In studies UC-1, UC-2, and UC-4, elevations of ALT to ≥3×ULN in at least one measurement were observed in 1.5% of patients treated with RINVOQ® 45 mg, and 0% of patients treated with placebo for 8 weeks. AST elevations to ≥3×ULN occurred in 1.5% of patients treated with RINVOQ® 45 mg, and 0.3% of patients treated with placebo. Elevations of ALT to ≥5×ULN occurred in 0.4% of patients treated with RINVOQ® 45 mg and 0% of patients treated with placebo.

In UC-3, elevations of ALT to ≥3×ULN in at least one measurement were observed in 4% of patients treated with RINVOQ® 30 mg, 2% of patients treated with RINVOQ® 15 mg, and 0.8% of patients treated with placebo for 52 weeks. Elevations of AST to ≥3×ULN in at least one measurement were observed in 2% of patients treated with RINVOQ® 30 mg, 1.6% of patients treated with RINVOQ® 15 mg and 0.4% of patients treated with placebo. Elevations of ALT to ≥5×ULN were observed in 0.8% of patients treated with 30 mg, 0.4% of patients treated with 15 mg, and 0.4% of patients treated with placebo.

Overall, laboratory abnormalities observed in patients with ulcerative colitis treated with RINVOQ® were similar to those described in patients with RA.

### Drug Interactions

### Strong CYP3A4 Inhibitors

Upadacitinib exposure is increased when RINVOQ® is co-administered with a strong CYP3A4 inhibitor (such as ketoconazole), which may increase the risk of RINVOQ® adverse reactions. Monitor patients closely for adverse reactions when co-administering RINVOQ® 15 mg once daily with strong CYP3A4 inhibitors.

For patients with atopic dermatitis, coadministration of RINVOQ® 30 mg once daily with strong CYP3A4 inhibitors is not recommended.

For patients with ulcerative colitis taking strong CYP3A4 inhibitors, reduce the RINVOQ® induction dosage to 30 mg once daily. The recommended maintenance dosage is 15 mg once daily.

### Strong CYP3A4 Inducers

Upadacitinib exposure is decreased when RINVOQ® is co-administered with strong CYP3A4 inducers (such as rifampin), which may lead to reduced therapeutic effect of RINVOQ®. Coadministration of RINVOQ® with strong CYP3A4 inducers is not recommended.

### Use in Specific Populations

### Pregnancy

### Risk Summary

Available data from the pharmacovigilance safety database and post-marketing case reports on use of RINVOQ® in pregnant women are not sufficient to evaluate a drug-associated risk for major birth defects or miscarriage. Based on animal studies, RINVOQ® has the potential to adversely affect a developing fetus. Advise patients of reproductive potential and pregnant patients of the potential risk to the fetus. In animal embryo-fetal development studies, oral upadacitinib administration to pregnant rats and rabbits at exposures equal to or greater than approximately 2 and 17 times the 15 mg dose and 0.9 and 8.5 times the maximum recommended human dose (MRHD) of 30 mg, respectively, resulted in dose-related increases in skeletal malformations (rats only), an increased incidence of cardiovascular malformations (rabbits only), increased post-implantation loss (rabbits only), and decreased fetal body weights in both rats and rabbits. No developmental toxicity was observed in pregnant rats and rabbits treated with oral upadacitinib during organogenesis at approximately 0.3 and 2.5 times the 15 mg dose and 0.2 and 1.3 times the MRHD of 30 mg, respectively. In a pre- and post-natal development study in pregnant female rats, oral upadacitinib administration at exposures approximately 1.6 times the MRHD of 30 mg resulted in no maternal or developmental toxicity (see Data).

The background risks of major birth defects and miscarriage for the indicated populations are unknown. All pregnancies have a background risk of birth defect, loss, or other adverse outcomes. In the U.S. general population, the estimated background risk of major birth defects and miscarriages are 2-4% and 15-20%, respectively.

### Clinical Considerations

Disease-Associated Maternal and/or Embryo/Fetal Risk

Published data suggest that increased disease activity is associated with the risk of developing adverse pregnancy outcomes in women with rheumatoid arthritis. Adverse pregnancy outcomes include preterm delivery (before 37 weeks of gestation), low birth weight (less than 2500 g) infants, and small for gestational age at birth.

### Data

### Animal Data

In an oral embryo-fetal development study, pregnant rats received upadacitinib at doses of 5, 25, and 75 mg/kg/day during the period of organogenesis from gestation day 6 to 17. Upadacitinib was teratogenic (skeletal malformations that consisted of misshapen humerus and bent scapula) at exposures equal to or greater than approximately 1.0 times the MRHD of 30 mg (on an AUC basis at maternal oral doses of 5 mg/kg/day and higher). Additional skeletal malformations (bent forelimbs/hindlimbs and rib/vertebral defects) and decreased fetal body weights were observed in the absence of maternal toxicity at an exposure approximately 48 times the MRHD of 30 mg (on an AUC basis at a maternal oral dose of 75 mg/kg/day).

In a second oral embryo-fetal development study, pregnant rats received upadacitinib at doses of 1.5 and 4 mg/kg/day during the period of organogenesis from gestation day 6 to 17. Upadacitinib was teratogenic (skeletal malformations that included bent humerus and scapula) at exposures approximately 0.9 times the MRHD of 30 mg (on an AUC basis at maternal oral doses of 4 mg/kg/day). No developmental toxicity was observed in rats at an exposure approximately 0.2 times the MRHD of 30 mg (on an AUC basis at a maternal oral dose of 1.5 mg/kg/day).

In an oral embryo-fetal developmental study, pregnant rabbits received upadacitinib at doses of 2.5, 10, and 25 mg/kg/day during the period of organogenesis from gestation day 7 to 19. Embryolethality, decreased fetal body weights, and cardiovascular malformations were observed in the presence of maternal toxicity at an exposure approximately 8.5 times the MRHD of 30 mg (on an AUC basis at a maternal oral dose of 25 mg/kg/day). Embryolethality consisted of increased post-implantation loss that was due to elevated incidences of both total and early resorptions. No developmental toxicity was observed in rabbits at an exposure approximately 1.3 times the MRHD of 30 mg (on an AUC basis at a maternal oral dose of 10 mg/kg/day).

In an oral pre- and post-natal development study, pregnant female rats received upadacitinib at doses of 2.5, 5, and 10 mg/kg/day from gestation day 6 through lactation day 20. No maternal or developmental toxicity was observed in either mothers or offspring, respectively, at an exposure approximately 1.6 times the MRHD of 30 mg (on an AUC basis at a maternal oral dose of 10 mg/kg/day).

### Lactation

### Risk Summary

There are no data on the presence of upadacitinib in human milk, the effects on the breastfed infant, or the effects on milk production. Available pharmacodynamic/toxicological data in animals have shown excretion of upadacitinib in milk. When a drug is present in animal milk, it is likely that the drug will be present in human milk. Because of the potential for serious adverse reactions in the breastfed infant, advise patients that breastfeeding is not recommended during treatment with RINVOQ®, and for 6 days (approximately 10 half-lives) after the last dose.

### Data

A single oral dose of 10 mg/kg radiolabeled upadacitinib was administered to lactating female Sprague-Dawley rats on post-partum days 7-8. Drug exposure was approximately 30-fold greater in milk than in maternal plasma based on AUC₀₋ₜ values. Approximately 97% of drug-related material in milk was parent drug.

### Females and Males of Reproductive Potential

### Pregnancy Testing

Verify the pregnancy status of females of reproductive potential prior to starting treatment with RINVOQ®.

### Contraception

### Females

Based on animal studies, upadacitinib may cause embryo-fetal harm when administered to pregnant women. Advise female patients of reproductive potential to use effective contraception during treatment with RINVOQ® and for 4 weeks after the final dose.

### Pediatric Use

### Juvenile Idiopathic Arthritis and Psoriatic Arthritis

The safety and effectiveness of RINVOQ® in pediatric patients with juvenile idiopathic arthritis and psoriatic arthritis have not been established.

### Atopic Dermatitis

The safety and effectiveness of RINVOQ® in pediatric patients 12 years of age and older weighing at least 40 kg with atopic dermatitis have been established. A total of 344 pediatric patients aged 12 to 17 years with moderate to severe atopic dermatitis were randomized across three trials (AD-1, AD-2 and AD-3) to receive either RINVOQ® 15 mg (N=114) or 30 mg (N=114) or matching placebo (N=116) in monotherapy or combination with topical corticosteroids. Efficacy was consistent between the pediatric patients and adults. The adverse reaction profile in the pediatric patients was similar to the adults. The safety and effectiveness of RINVOQ® in pediatric patients less than 12 years of age with atopic dermatitis have not been established.

### Ulcerative Colitis

The safety and effectiveness of RINVOQ® in pediatric patients with ulcerative colitis have not been established.

### Geriatric Use

### Rheumatoid Arthritis and Psoriatic Arthritis

Of the 4381 patients treated in the five clinical studies, a total of 906 rheumatoid arthritis patients were 65 years of age or older, including 146 patients 75 years and older. Of the 1827 patients treated in the two psoriatic arthritis Phase 3 clinical studies, a total of 274 patients were 65 years of age or older, including 34 patients 75 years and older. No differences in effectiveness were observed between these patients and younger patients; however, there was a higher rate of overall adverse events, including serious infections, in patients 65 years of age and older.

### Atopic Dermatitis

Of the 2583 patients treated in the three Phase 3 clinical trials, a total of 120 patients with atopic dermatitis were 65 years of age or older, including 6 patients 75 years of age. No differences in effectiveness were observed between these patients and younger patients; however, there was a higher rate of serious infections and malignancies in those patients 65 years of age or older in the 30 mg dosing group in the long-term trials.

### Ulcerative Colitis

Of the 1097 patients treated in the controlled clinical trials, a total of 95 patients with ulcerative colitis were 65 years and older. Clinical studies of RINVOQ® did not include sufficient numbers of patients 65 years of age and older with ulcerative colitis to determine whether they respond differently from younger adult patients.

### Renal Impairment

For patients with rheumatoid arthritis and psoriatic arthritis, no dosage adjustment is needed in patients with mild (eGFR 60 to <90 mL/min/1.73 m²), moderate (eGFR 30 to <60 mL/min/1.73 m²), or severe renal impairment (eGFR 15 to <30 mL/min/1.73 m²).

For patients with atopic dermatitis, the maximum recommended dosage is 15 mg once daily for patients with severe renal impairment. No dosage adjustment is needed in patients with mild or moderate renal impairment.

For patients with ulcerative colitis, the recommended dosage for severe renal impairment is 30 mg once daily (for up to 16 weeks) for induction and 15 mg once daily for maintenance. No dosage adjustment is needed in patients with mild or moderate renal impairment.

RINVOQ® has not been studied in patients with end stage renal disease (eGFR <15 mL/min/1.73 m²). Use in patients with atopic dermatitis or ulcerative colitis with end stage renal disease is not recommended.

### Hepatic Impairment

The use of RINVOQ® has not been studied in patients with severe hepatic impairment (Child Pugh C), and therefore not recommended for use in patients with rheumatoid arthritis, psoriatic arthritis, atopic dermatitis or ulcerative colitis.

For patients with rheumatoid arthritis, psoriatic arthritis, and atopic dermatitis, no dosage adjustment is needed in patients with mild (Child Pugh A) or moderate (Child Pugh B) hepatic impairment.

For patients with ulcerative colitis, the recommended dosage for mild to moderate hepatic impairment is 30 mg once daily for induction and 15 mg once daily for maintenance.

### Description

RINVOQ® is formulated with upadacitinib, a JAK inhibitor. Upadacitinib has the following chemical name: (3S,4R)-3-Ethyl-4-(3H-imidazo[1,2-a]pyrrolo[2,3-e]pyrazin-8-yl)-N-(2,2,2-trifluoroethyl)pyrrolidine-1-carboxamide hydrate (2:1). The strength of upadacitinib is based on anhydrous upadacitinib. The solubility of upadacitinib in water is 38 to less than 0.2 mg/mL across a pH range of 2 to 9 at 37° C. Upadacitinib has a molecular weight of 389.38 g/mol and a molecular formula of C₁₇H₁₉F₃N₆O·½H2O. The chemical structure of upadacitinib is:

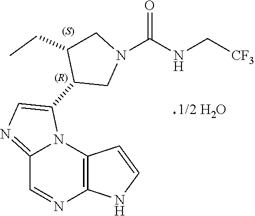

RINVOQ® 15 mg extended-release tablets for oral administration are purple, biconvex oblong, with dimensions of 14×8 mm, and debossed with ‘a15’ on one side. Each tablet contains the following inactive ingredients: colloidal silicon dioxide, ferrosoferric oxide, hypromellose, iron oxide red, magnesium stearate, mannitol, microcrystalline cellulose, polyvinyl alcohol, polyethylene glycol, talc, tartaric acid and titanium dioxide.

RINVOQ® 30 mg extended-release tablets for oral administration are red, biconvex oblong, with dimensions of 14×8 mm, and debossed with ‘a30’ on one side. Each tablet contains the following inactive ingredients: colloidal silicon dioxide, hypromellose, iron oxide red, magnesium stearate, mannitol, microcrystalline cellulose, polyvinyl alcohol, polyethylene glycol, talc, tartaric acid and titanium dioxide.

RINVOQ® 45 mg extended-release tablets for oral administration are yellow to mottled yellow, biconvex oblong, with dimensions of 14×8 mm, and debossed with ‘a45’ on one side. Each tablet contains the following inactive ingredients: colloidal silicon dioxide, hypromellose, iron oxide yellow, iron oxide red, magnesium stearate, mannitol, microcrystalline cellulose, polyvinyl alcohol, polyethylene glycol, talc, tartaric acid and titanium dioxide.

### Clinical Pharmacology

### Mechanism of Action

Upadacitinib is a Janus kinase (JAK) inhibitor. JAKs are intracellular enzymes which transmit signals arising from cytokine or growth factor-receptor interactions on the cellular membrane to influence cellular processes of hematopoiesis and immune cell function. Within the signaling pathway, JAKs phosphorylate and activate signal transducers and activators of transcription (STATs) which modulate intracellular activity including gene expression. Upadacitinib modulates the signaling pathway at the point of JAKs, preventing the phosphorylation and activation of STATs.

JAK enzymes transmit cytokine signaling through their pairing (e.g., JAK1/JAK2, JAK1/JAK3, JAK1/TYK2, JAK2/JAK2, JAK2/TYK2). In a cell-free isolated enzyme assay, upadacitinib had greater inhibitory potency at JAK1 and JAK2 relative to JAK3 and TYK2. In human leukocyte cellular assays, upadacitinib inhibited cytokine-induced STAT phosphorylation mediated by JAK1 and JAK1/JAK3 more potently than JAK2/JAK2 mediated STAT phosphorylation. However, the relevance of inhibition of specific JAK enzymes to therapeutic effectiveness is not currently known.

### Pharmacodynamics

### Inhibition of IL-6 Induced STAT3 and IL-7 Induced STATS Phosphorylation

In healthy volunteers, the administration of upadacitinib (immediate release formulation) resulted in a dose- and concentration-dependent inhibition of IL-6 (JAK1/JAK2)-induced STAT3 and IL-7 (JAK1/JAK3)-induced STATS phosphorylation in whole blood. The maximal inhibition was observed 1 hour after dosing which returned to near baseline by the end of dosing interval.

### Lymphocytes

In patients with rheumatoid arthritis, treatment with upadacitinib was associated with a small, transient increase in mean ALC from baseline up to Week 36 which gradually returned to, at or near baseline levels with continued treatment.

### Immunoglobulins

In patients with rheumatoid arthritis, small decreases from baseline in mean IgG and IgM levels were observed with upadacitinib treatment in the controlled period; however, the mean values at baseline and at all visits were within the normal reference range.

### Cardiac Electrophysiology

At 2.5 times the mean exposure of the maximum therapeutic dose, 45 mg once daily dose, there was no clinically relevant effect on the QTc interval.

### Pharmacokinetics

Upadacitinib plasma exposures are proportional to dose over the therapeutic dose range. After a single dose administration of RINVOQ 15 mg, 30 mg, and 45 mg under fasting condition in healthy subjects, mean Cₘₐₓ was 31.6 ng/mL, 71.8 ng/mL, and 90.7 ng/mL, respectively, and mean AUCₘf was 265 ng·h/mL, 543 ng·h/mL, and 752 ng·h/mL, respectively. Steady-state plasma concentrations are achieved within 4 days with minimal accumulation after once daily administration. Upadacitinib pharmacokinetics are comparable between rheumatoid arthritis, psoriatic arthritis, atopic dermatitis, and ulcerative colitis patients.

### Absorption

Following oral administration of upadacitinib extended-release formulation, upadacitinib is absorbed with a median Tₘₐₓ of 2 to 4 hours.

Coadministration of upadacitinib with a high-fat/high-calorie meal had no clinically relevant effect on upadacitinib exposures (increased AUC_{inf} by 29% and Cmax by 39% to 60%). In clinical trials, upadacitinib was administered without regard to meals.

### Distribution

Upadacitinib is 52% bound to plasma proteins. Upadacitinib partitions similarly between plasma and blood cellular components with a blood to plasma ratio of 1.0.

### Elimination

### Metabolism

Upadacitinib metabolism is mediated by mainly CYP3A4 with a potential minor contribution from CYP2D6. The pharmacologic activity of upadacitinib is attributed to the parent molecule. In a human radiolabeled study, unchanged upadacitinib accounted for 79% of the total radioactivity in plasma while the main metabolite detected (product of monooxidation followed by glucuronidation) accounted for 13% of the total plasma radioactivity. No active metabolites have been identified for upadacitinib.

### Excretion

Following single dose administration of [14C]-upadacitinib immediate-release solution, upadacitinib was eliminated predominantly as the unchanged parent drug in urine (24%) and feces (38%). Approximately 34% of upadacitinib dose was excreted as metabolites. Upadacitinib mean terminal elimination half-life ranged from 8 to 14 hours.

### Specific Populations

### Body Weight, Gender, and Race

Body weight, gender, race, ethnicity, and ethnicity did not have a clinically meaningful effect on upadacitinib exposure.

### Pediatric Patients

No meaningful difference in the systemic exposure of upadacitinib was observed in pediatric patients with atopic dermatitis 12 years of age and older weighing at least 40 kg compared to adults.

### Geriatric Patients

No clinically meaningful differences in the pharmacokinetics of upadacitinib were observed in geriatric patients (>65 years of age) compared to younger adult patients.

Patients with Renal Impairment

Upadacitinib mean AUC_{inf} after a single dose administration of 15 mg upadacitinib was 18%, 33%, and 44% higher in patients with mild (eGFR 60 to <90 mL/min/1.73 m²), moderate (eGFR 30 to <60 mL/min/1.73 m²), and severe renal impairment (eGFR 15 to <30 mL/min/1.73 m²), respectively, compared to subjects with normal renal function (eGFR ≥90 mL/min/1.73 m²). Upadacitinib mean Cₘₐₓ was similar among subjects with normal and impaired renal function. In patients receiving 15 mg, 30 mg, or 45 mg once daily upadacitinib, mild and moderate renal impairment is not expected to have a clinically relevant effect on upadacitinib exposure.

Patients with Hepatic Impairment

Upadacitinib mean AUC_{inf} after a single dose administration of 15 mg upadacitinib was 28% and 24% higher in patients with mild (Child-Pugh A) and moderate (Child-Pugh B) hepatic impairment, respectively, compared to subjects with normal liver function. Upadacitinib mean Cₘₐₓ was unchanged in patients with mild hepatic impairment and 43% higher in patients with moderate hepatic impairment compared to subjects with normal liver function. Upadacitinib was not studied in patients with severe hepatic impairment (Child-Pugh C)

### Drug Interaction Studies

### Potential for Other Drugs to Influence the Pharmacokinetics of Upadacitinib

Upadacitinib is metabolized in vitro by CYP3A4 with a minor contribution from CYP2D6. The effect of co-administered drugs on upadacitinib plasma exposures is provided in Table 22.

TABLE 22
Change in Pharmacokinetics of Upadacitinib in the Presence of Co-administered Drugs

| | Regimen of | Ratio (90% CI)^{a} | |
|---|---|---|---|
| Co-administered Drug | Co-administered Drug | Cₘₐₓ | AUC |
| Methotrexate | 10 to 25 mg/week | 0.97 (0.86-1.09) | 0.99 (0.93-1.06) |
| Strong CYP3A4 inhibitor: Ketoconazole | 400 mg once daily x 6 days | 1.70 (1.55-1.89) | 1.75 (1.62-1.88) |
| Strong CYP3A4 inducer: Rifampin | 600 mg once daily x 9 days | 0.49 (0.44-0.55) | 0.39 (0.37-0.42) |
| OATP1B inhibitor: Rifampin | 600 mg single dose | 1.14 (1.02-1.28) | 1.07 (1.01-1.14) |

CI: Confidence interval
^{a}Ratios for Cₘₐₓ and AUC compare co-administration of the medication with upadacitinib vs. administration of upadacitinib alone.

pH modifying medications (e.g., antacids or proton pump inhibitors) are not expected to affect upadacitinib plasma exposures based on in vitro assessments and population pharmacokinetic analyses. CYP2D6 metabolic phenotype had no effect on upadacitinib pharmacokinetics (based on population pharmacokinetic analyses), indicating that inhibitors of CYP2D6 have no clinically relevant effect on upadacitinib exposures.

### Potential for Upadacitinib to Influence the Pharmacokinetics of Other Drugs

In vitro studies indicate that upadacitinib does not inhibit or induce the activity of cytochrome P450 (CYP) enzymes (CYP1A2, CYP2B6, CYP2C8, CYP2C9, CYP2C19, CYP2D6, and CYP3A4) at clinically relevant concentrations. In vitro studies indicate that upadacitinib does not inhibit the transporters P-gp, BCRP, OATP1B1, OATP1B3, OCT1, OCT2, OAT1, OAT3, MATE1, and MATE2K at clinically relevant concentrations.

Clinical studies indicate that upadacitinib has no clinically relevant effects on the pharmacokinetics of co-administered drugs. Following upadacitinib 30 mg and 45 mg once daily, the effects on each CYP enzymes (CYP1A2, CYP3A, CYP2C9, and CYP2C19) were similar between two doses except for the effect on CYP2D6. Following upadacitinib 30 mg and 45 mg once daily, a weak induction of CYP3A4 was observed. A weak inhibition of CYP2D6 was observed at upadacitinib 45 mg but not at 30 mg. Summary of results from clinical studies which evaluated the effect of upadacitinib on other drugs is provided in Table 23.

TABLE 23
Change in Pharmacokinetics of Co-administered Drugs or In Vivo
Markers of CYP Activity in the Presence of Upadacitinib

| Co-administered Drug | Multiple-Dose Regimen of | Ratio (90% CI)^{a} | |
|---|---|---|---|
| or CYP Activity Marker | Upadacitinib | Cₘₐₓ | AUC |
| Methotrexate | 6 mg to 24 mg BID^{b} | 1.03 (0.86-1.23) | 1.14 (0.91-1.43) |
| Sensitive CYP1A2 Substrate: Caffeine | 45 mg QD^{c} | 1.05 (0.97-1.14) | 1.04 (0.95-1.13) |
| Sensitive CYP3A Substrate: Midazolam | 30 mg QD^{c} | 0.74 (0.68-0.80) | 0.74 (0.68-0.80) |
| Sensitive CYP3A Substrate: Midazolam | 45 mg QD^{c} | 0.75 (0.69-0.83) | 0.76 (0.69-0.83) |
| Sensitive CYP2D6 Substrate: Dextromethorphan | 30 mg QD^{c} | 1.09 (0.98-1.21) | 1.07 (0.95-1.22) |
| Sensitive CYP2D6 Substrate: Dextromethorphan | 45 mg QD^{c} | 1.30 (1.13-1.50) | 1.35 (1.18-1.54) |
| Sensitive CYP2C9 Substrate: S-Warfarin | 45 mg QD^{c} | 1.18 (1.05-1.33) | 1.12 (1.05-1.20) |
| Sensitive CYP2C19 Marker: 5-OH | 45 mg QD^{c} | — | 0.96 (0.90-1.02) |
| Omeprazole to Omeprazole metabolic ratio | | | |
| CYP2B6 Substrate: Bupropion | 30 mg QD^{c} | 0.87 (0.79-0.96) | 0.92 (0.87-0.98) |
| Rosuvastatin | 30 mg QD^{c} | 0.77 (0.63-0.94) | 0.67 (0.56-0.82) |
| Atorvastatin | 30 mg QD^{c} | 0.88 (0.79-0.97) | 0.77 (0.70-0.85) |
| Ethinylestradiol | 30 mg QD^{c} | 0.96 (0.89-1.02) | 1.11 (1.04-1.19) |
| Levonorgestrel | 30 mg QD^{c} | 0.96 (0.87-1.06) | 0.96 (0.85-1.07) |

CYP: cytochrome P450;
CI: Confidence interval;
BID: twice daily;
QD: once daily
^{a}Ratios for Cₘₐₓ and AUC compare co-administration of the medication with upadacitinib vs. administration of medication alone.
^{b}Immediate-release formulation
^{c}Extended-release formulation

### 13 Nonclinical Toxicology

### 13.1 Carcinogenesis, Mutagenesis, Impairment of Fertility

### Carcinogenesis

The carcinogenic potential of upadacitinib was evaluated in Sprague-Dawley rats and Tg.rasH2 mice. No evidence of tumorigenicity was observed in male or female rats that received upadacitinib for up to 101 weeks at oral doses up to 15 or 20 mg/kg/day, respectively (approximately 2.4 and 6.0 times the MRHD of 30 mg on an AUC basis, respectively). No evidence of tumorigenicity was observed in male or female Tg.rasH2 mice that received upadacitinib for 26 weeks at oral doses up to 20 mg/kg/day.

### Mutagenesis

Upadacitinib tested negatively in the following genotoxicity assays: the in vitro bacterial mutagenicity assay (Ames assay), in vitro chromosome aberration assay in human peripheral blood lymphocytes, and in vivo rat bone marrow micronucleus assay.

### Impairment of Fertility

Upadacitinib had no effect on fertility in male or female rats at oral doses up to 50 mg/kg/day in males and 75 mg/kg/day in females (approximately 24 and 48 times the MRHD of 30 mg in males and females, respectively, on an AUC basis). However, maintenance of pregnancy was adversely affected at oral doses of 25 mg/kg/day and 75 mg/kg/day based upon dose-related findings of increased post-implantation losses (increased resorptions) and decreased numbers of mean viable embryos per litter (approximately 13 and 48 times the MRHD of 30 mg on an AUC basis, respectively). The number of viable embryos was unaffected in female rats that received upadacitinib at an oral dose of 5 mg/kg/day and were mated to males that received the same dose (approximately 1.0 times the MRHD of 30 mg on an AUC basis).

### Clinical Studies

### Rheumatoid Arthritis

The efficacy and safety of RINVOQ® 15 mg once daily were assessed in five Phase 3 randomized, double-blind, multicenter trials in patients with moderately to severely active rheumatoid arthritis and fulfilling the ACR/EULAR 2010 classification criteria. Patients 18 years of age and older were eligible to participate. The presence of at least 6 tender and 6 swollen joints and evidence of systemic inflammation based on elevation of hsCRP was required at baseline. Although other doses have been studied, the recommended dosage of RINVOQ® is 15 mg once daily.

Trial RA-I (NCT02706873) was a 24-week monotherapy trial in 947 patients with moderately to severely active rheumatoid arthritis who were naïve to methotrexate (MTX). Patients received RINVOQ® 15 mg or upadacitinib 30 mg orally once daily or MTX as monotherapy. At Week 26, non-responding patients on upadacitinib could be rescued with the addition of MTX, while patients on MTX could be rescued with the addition of blinded RINVOQ® 15 mg or upadacitinib 30 mg once daily. The primary endpoint was the proportion of patients who achieved an ACR50 response at Week 12. Key secondary endpoints included disease activity score (DAS28-CRP) ≤3.2 at Week 12, DAS28-CRP <2.6 at Week 24, change from baseline in HAQ-DI at Week 12, and change from baseline in van der Heijde-modified total Sharp Score (mTSS) at Week 24.

Trial RA-II (NCT02706951) was a 14-week monotherapy trial in 648 patients with moderately to severely active rheumatoid arthritis who had an inadequate response to MTX. Patients received RINVOQ® 15 mg or upadacitinib 30 mg once daily monotherapy or continued their stable dose of MTX monotherapy. At Week 14, patients who were randomized to MTX were advanced to RINVOQ® 15 mg or upadacitinib 30 mg once daily monotherapy in a blinded manner based on pre-determined assignment at baseline. The primary endpoint was the proportion of patients who achieved an ACR20 response at Week 14. Key secondary endpoints included DAS28-CRP ≤3.2, DAS28-CRP <2.6, and change from baseline in HAQ-DI at Week 14.

Trial RA-III (NCT02675426) was a 12-week trial in 661 patients with moderately to severely active rheumatoid arthritis who had an inadequate response to conventional disease modifying anti-rheumatic drugs (cDMARDs). Patients received RINVOQ® 15 mg or upadacitinib 30 mg once daily or placebo added to background cDMARD therapy. At Week 12, patients who were randomized to placebo were advanced to RINVOQ® 15 mg or upadacitinib 30 mg once daily in a blinded manner based on pre-determined assignment at baseline. The primary endpoint was the proportion of patients who achieved an ACR20 response at Week 12. Key secondary endpoints included DAS28-CRP ≤3.2, DAS28-CRP<2.6, and change from baseline in HAQ-DI at Week 12.

Trial RA-IV (NCT02629159) was a 48-week trial in 1629 patients with moderately to severely active rheumatoid arthritis who had an inadequate response to MTX. Patients received RINVOQ® 15 mg once daily, active comparator, or placebo added to background MTX. From Week 14, non-responding patients on RINVOQ® 15 mg could be rescued to active comparator in a blinded manner, and non-responding patients on active comparator or placebo could be rescued to RINVOQ® 15 mg in a blinded manner. At Week 26, all patients randomized to placebo were switched to RINVOQ® 15 mg once daily in a blinded manner. The primary endpoint was the proportion of patients who achieved an ACR20 response at Week 12 versus placebo. Key secondary endpoints versus placebo included DAS28-CRP ≤3.2, DAS28-CRP <2.6, change from baseline in HAQ-DI at Week 12, and change from baseline in mTSS at Week 26.

Trial RA-V (NCT02706847) was a 12-week trial in 499 patients with moderately to severely active rheumatoid arthritis who had an inadequate response or intolerance to biologic DMARDs. Patients received RINVOQ® 15 mg or upadacitinib 30 mg once daily or placebo added to background cDMARD therapy. At Week 12, patients who were randomized to placebo were advanced to RINVOQ® 15 mg or upadacitinib 30 mg once daily in a blinded manner based on pre-determined assignment at baseline. The primary endpoint was the proportion of patients who achieved an ACR20 response at Week 12. Key secondary endpoints included DAS28-CRP <3.2 and change from baseline in HAQ-DI at Week 12.

### Clinical Response

The percentages of RINVOQ®-treated patients achieving ACR20, ACR50, and ACR70 responses, and DAS28(CRP) <2.6 in all trials are shown in Table 24. Patients treated with RINVOQ® 15 mg, alone or in combination with cDMARDs, achieved higher ACR response rates compared to MTX monotherapy or placebo, respectively, at the primary efficacy timepoint (Table 24). In Trial IV, the percent of patients achieving ACR20 response by visit is shown in FIG. 1. In Trials RA-III and RA-V, higher ACR20 response rates were observed at 1 week with RINVOQ® 15 mg versus placebo. Treatment with RINVOQ® 15 mg, alone or in combination with cDMARDs, resulted in greater improvements in the ACR components compared to MTX or placebo at the primary efficacy timepoint (Table 25).

TABLE 24
Clinical Response in RA Patients in Trials RA-I, RA-II, RA-III, RA-IV and RA V

| | | | | | Trial RA-III | | Trial RA-IV | | Trial RA-V | |
|---|---|---|---|---|---|---|---|---|---|---|
| | Trial RA-I | | Trial RA-II | | cDMARD-IR | | MTX-IR | | bDMARD-IR | |
| | MTX-Naïve | | MTX-IR | | Background | | Background | | Background | |
| | Monotherapy | | Monotherapy | | cDMARDs | | MTX | | cDMARDs | |
| | | RINVOQ ® | | RINVOQ ® | | RINVOQ ® | | RINVOQ ® | | RINVOQ ® |
| | | 15 mg | | 15 mg | | 15 mg | | 15 mg | | 15 mg |
| | | % | | % | | % | | % | | % |
| | MTX | Δ (95% CI) | MTX | Δ (95% CI) | PBO | Δ (95% CI) | PBO | Δ (95% CI) | PBO | Δ (95% CI) |
| N | 314 | 317 | 216 | 217 | 221 | 221 | 651 | 651 | 169 | 164 |
| Week | | | | | | | | | | |

ACR20

| | | | | | | | | | | |
|---|---|---|---|---|---|---|---|---|---|---|
| 12^{a}/14^{b} | 54 | 76 | 41 | 68 | 36 | 64 | 36 | 71 | 28 | 65 |
| | | 22 (14, 29) | | 26 (17, 36) | | 28 (19, 37) | | 34 (29, 39) | | 36 (26, 46) |
| 24^{c}/26^{d} | 59 | 79 | | | | | 36 | 67 | | |
| | | 20 (13, 27) | | | | | | 32 (27, 37) | | |

ACR50

| | | | | | | | | | | |
|---|---|---|---|---|---|---|---|---|---|---|
| 12^{a}/14^{b} | 28 | 52 | 15 | 42 | 15 | 38 | 15 | 45 | 12 | 34 |
| | | 24 (16, 31) | | 27 (18, 35) | | 23 (15, 31) | | 30 (26, 35) | | 22 (14, 31) |
| 24^{c}/26^{d} | 33 | 60 | | | | | 21 | 54 | | |
| | | 27 (19, 34) | | | | | | 33 (28, 38) | | |

ACR70

| | | | | | | | | | | |
|---|---|---|---|---|---|---|---|---|---|---|
| 12^{a}/14^{b} | 14 | 32 | 3 | 23 | 6 | 21 | 5 | 25 | 7 | 12 |
| | | 18 (12, 25) | | 20 (14, 26) | | 15 (9, 21) | | 20 (16, 24) | | 5 (−1, 11) |
| 24^{c}/26^{d} | 18 | 44 | | | | | 10 | 35 | | |
| | | 26 (19, 33) | | | | | | 25 (21, 29) | | |

DAS28-CRP <2.6

| | | | | | | | | | | |
|---|---|---|---|---|---|---|---|---|---|---|
| 12^{a}/14^{b} | 14 | 36 | 8 | 28 | 10 | 31 | 6 | 29 | 9 | 29 |
| | | 22 (15, 28) | | 20 (13, 27) | | 21 (14, 28) | | 23 (19, 27) | | 19 (11,27) |
| 24^{c}/26^{d} | 18 | 48 | | | | | 9 | 41 | | |
| | | 30 (23, 37) | | | | | | 32 (27, 36) | | |

Abbreviations:
ACR20 (or 50 or 70) = American College of Rheumatology ≥20% (or >50% or ≥70%) improvement;
bDMARD = biologic disease-modifying anti-rheumatic drug;
CRP = c-reactive protein;
DAS28 = Disease Activity Score 28 joints;
cDMARDs = conventional disease-modifying anti-rheumatic drugs;
MTX = methotrexate;
PBO = placebo;
IR = inadequate responder
Patients who discontinued randomized treatment, or had cross-over between randomized treatments, or were missing data at week of evaluation were imputed as non-responders in the analyses.
^{a}Trial RA-I, Trial RA-III, Trial RA-IV, Trial RA-V
^{b}Trial RA-II
^{c}Trial A-I
^{d}Trial RA-IV

TABLE 25
Components of ACR Response at Primary Efficacy Timepoint^{a}

| | | | | | Trial RA-III | | Trial RA-IV | | Trial RA-V | |
|---|---|---|---|---|---|---|---|---|---|---|
| | Trial RA-I | | Trial RA-II^{b} | | cDMARD-IR | | MTX-IR | | bDMARD-IR | |
| | MTX-Naïve | | MTX-IR | | Background | | Background | | Background | |
| | Monotherapy | | Monotherapy | | cDMARDs | | MTX | | cDMARDs | |
| | | RINVOQ ® | | RINVOQ ® | | RINVOQ ® | | RINVOQ ® | | RINVOQ ® |
| | MTX | 15 mg | MTX | 15 mg | PBO | 15 mg | PBO | 15 mg | PBO | 15 mg |
| N | 314 | 317 | 216 | 217 | 221 | 221 | 651 | 651 | 169 | 164 |

Number of tender joints (0-68)

| | | | | | | | | | | |
|---|---|---|---|---|---|---|---|---|---|---|
| Baseline | 26 | 25 | 25 | 24 | 25 | 25 | 26 | 26 | 28 | 28 |
| | (16) | (14) | (16) | (15) | (15) | (14) | (14) | (15) | (15) | (16) |
| Week | 13 | 9 | 15 | 10 | 16 | 12 | 16 | 10 | 18 | 11 |
| 12/14 | (15) | (12) | (16) | (13) | (17) | (14) | (15) | (13) | (17) | (14) |

Number of swollen joints (0-66)

| | | | | | | | | | | |
|---|---|---|---|---|---|---|---|---|---|---|
| Baseline | 17 | 17 | 17 | 16 | 15 | 16 | 16 | 17 | 16 | 17 |
| | (11) | (10) | (12) | (11) | (9) | (10) | (9) | (10) | (10) | (11) |
| Week | 6 | 5 | 9 | 6 | 9 | 7 | 9 | 5 | 9 | 6 |
| 12/14 | (8) | (7) | (11) | (9) | (10) | (10) | (9) | (7) | (10) | (8) |

Pain^{c}

| | | | | | | | | | | |
|---|---|---|---|---|---|---|---|---|---|---|
| Baseline | 66 | 68 | 63 | 62 | 62 | 64 | 65 | 66 | 69 | 68 |
| | (21) | (21) | (21) | (23) | (21) | (19) | (21) | (21) | (21) | (20) |
| Week | 41 | 31 | 49 | 36 | 51 | 33 | 49 | 33 | 55 | 41 |
| 12/14 | (25) | (25) | (25) | (27) | (26) | (24) | (25) | (24) | (28) | (28) |

Patient global assessment^{c}

| | | | | | | | | | | |
|---|---|---|---|---|---|---|---|---|---|---|
| Baseline | 66 | 67 | 60 | 62 | 60 | 63 | 64 | 64 | 66 | 67 |
| | (21) | (22) | (22) | (22) | (20) | (22) | (21) | (22) | (23) | (20) |
| Week | 42 | 31 | 48 | 37 | 50 | 32 | 48 | 33 | 54 | 40 |
| 12/14 | (25) | (24) | (26) | (27) | (26) | (24) | (24) | (24) | (28) | (26) |

Disability Index (HAQ-DI)^{d}

| | | | | | | | | | | |
|---|---|---|---|---|---|---|---|---|---|---|
| Baseline | 1.60 | 1.60 | 1.47 | 1.47 | 1.42 | 1.48 | 1.61 | 1.63 | 1.56 | 1.67 |
| | (0.67) | (0.67) | (0.66) | (0.66) | (0.63) | (0.61) | (0.61) | (0.64) | (0.60) | (0.64) |
| Week | 1.08 | 0.76 | 1.19 | 0.86 | 1.13 | 0.85 | 1.28 | 0.98 | 1.33 | 1.24 |
| 12/14 | (0.72) | (0.69) | (0.69) | (0.67) | (0.70) | (0.66) | (0.67) | (0.68) | (0.66) | (0.77) |

Physician global assessment^{c}

| | | | | | | | | | | |
|---|---|---|---|---|---|---|---|---|---|---|
| Baseline | 69 | 67 | 62 | 66 | 64 | 64 | 66 | 66 | 67 | 69 |
| | (16) | (17) | (17) | (18) | (18) | (16) | (18) | (17) | (17) | (17) |
| Week | 32 | 22 | 37 | 26 | 41 | 26 | 41 | 27 | 39 | 29 |
| 12/14 | (22) | (19) | (24) | (21) | (24) | (21) | (25) | (21) | (25) | (22) |

CRP (mg/L)

| | | | | | | | | | | |
|---|---|---|---|---|---|---|---|---|---|---|
| Baseline | 21.2 | 23.0 | 14.5 | 14.0 | 12.6 | 16.6 | 18.0 | 17.9 | 16.3 | 16.3 |
| | (22.1) | (27.4) | (17.3) | (16.5) | (14.0) | (19.2) | (21.5) | (22.5) | (21.1) | (18.6) |
| Week | 10.9 | 4.2 | 12.8 | 3.7 | 13.1 | 4.6 | 16.2 | 5.5 | 13.9 | 5.0 |
| 12/14 | (14.9) | (8.8) | (21.4) | (7.8) | (15.5) | (9.6) | (19.8) | (10.9) | (17.3) | (14.0) |

Abbreviations:
ACR = American College of Rheumatology;
bDMARD = biologic disease-modifying anti-rheumatic drug;
CRP = c-reactive protein;
cDMARDs = conventional disease-modifying anti-rheumatic drugs;
HAQ-DI = Health Assessment Questionnaire Disability Index;
IR = inadequate responder;
MTX = methotrexate;
PBO = placebo
^{a}Data shown are mean (standard deviation).
^{b}Primary efficacy timepoint is at Week 14.
^{c}Visual analog scale: 0 = best, 100 = worst.
^{d}Health Assessment Questionnaire-Disability Index: 0 = best, 3 = worst; 20 questions; 8 categories: dressing and grooming, arising, eating, walking, hygiene, reach, grip, and activities.

In RA-I and RA-IV, a higher proportion of patients treated with RINVOQ® 15 mg alone or in combination with MTX, achieved DAS28-CRP <2.6 compared to MTX or placebo at the primary efficacy timepoint (Table 26).

TABLE 26
Proportion of Patients with DAS28-CRP Less Than 2.6 with Number
of Residual Active Joints at Primary Efficacy Timepoint

| | Trial RA-I MTX-Naive | | |
|---|---|---|---|
| | Monotherapy | | |
| | MTX | | RINVOQ ® 15 mg |
| DAS28-CRP Less Than 2.6 | N = 314 | | N = 317 |
| Proportion of responders at Week 12 (n) | 14% | (43) | 36% (113) |
| Of responders, proportion with 0 active joints (n) | 51% | (22) | 45% (51) |
| Of responders, proportion with 1 active joint (n) | 35% | (15) | 23% (26) |
| Of responders, proportion with 2 active joints (n) | 9% | (4) | 17% (19) |
| Of responders, proportion with 3 or more active joints (n) | 5% | (2) | 15% (17) |
| | Trial RA-IV MTX-IR | | |
| | Background MTX | | |
| | PBO | | RINVOQ ® 15 mg |
| DAS28-CRP Less Than 2.6 | N = 651 | | N = 651 |
| Proportion of responders at Week 12 (n) | 6% | (40) | 29% (187) |
| Of responders, proportion with 0 active joints (n) | 60% | (24) | 48% (89) |
| Of responders, proportion with 1 active joint (n) | 20% | (8) | 23% (43) |
| Of responders, proportion with 2 active joints (n) | 15% | (6) | 13% (25) |
| Of responders, proportion with 3 or more active joints (n) | 5% | (2) | 16% (30) |

Abbreviations:
CRP = c-reactive protein;
DAS28 = Disease Activity Score 28 joints;
MTX = methotrexate;
PBO = placebo;
IR = inadequate responder

### Radiographic Response

Inhibition of progression of structural joint damage was assessed using the modified Total Sharp Score (mTSS) and its components, the erosion score and joint space narrowing score, at Week 26 in Trial RA-IV and Week 24 in Trial RA-I. The proportion of patients with no radiographic progression (mTSS change from baseline ≤0) was also assessed.

In Trial RA-IV, treatment with RINVOQ® 15 mg inhibited the progression of structural joint damage compared to placebo in combination with cDMARDs at Week 26 (Table 27). Analyses of erosion and joint space narrowing scores were consistent with overall results.

In the placebo plus MTX group, 76% of the patients experienced no radiographic progression at Week 26 compared to 83% of the patients treated with RINVOQ® 15 mg.

In Trial RA-I, treatment with RINVOQ® 15 mg monotherapy inhibited the progression of structural joint damage compared to MTX monotherapy at Week 24 (Table 27). Analyses of erosion and joint space narrowing scores were consistent with overall results.

In the MTX monotherapy group, 78% of the patients experienced no radiographic progression at Week 24 compared to 87% of the patients treated with RINVOQ® 15 mg monotherapy.

TABLE 27
Radiographic Changes

| | Trial RA-IV MTX-IR | | |
|---|---|---|---|
| | Background MTX | | |
| | PBO | RINVOQ ® 15 mg | Estimated Difference |
| | (N = 651) | (N = 651) | vs PBO at Week 26 |
| mTSS | Mean (SD) | Mean (SD) | (95% CI)^{a} |
| Baseline | 35.9 (52) | 34.0 (50) | |
| Week 26^{b} | 0.78 (0.1) | 0.15 (0.1) | −0.63 (−0.92, −0.34) |
| | Trial RA-I MTX-naïve | | |
| | Monotherapy | | |
| | MTX | RINVOQ ® 15 mg | Estimated Difference |
| | (N = 309) | (N = 309) | vs MTX at Week 24 |
| | Mean (SD) | Mean (SD) | (95% CI)^{c} |
| Baseline | 13.3 (31) | 18.1 (38) | |
| Week 24^{d} | 0.67 (2.8) | 0.14 (1.4) | −0.53 (−0.85, −0.20) |

Abbreviations:
mTSS = modified Total Sharp Score,
MTX = methotrexate;
PBO = placebo;
SD = standard deviation;
IR = inadequate responders;
bDMARDs = biologic disease modifying anti-rheumatic drugs;
LS = least squares;
CI = confidence intervals
^{a}LS means and 95% CI based on a random coefficient model fit to the mTSS value adjusting for time, treatment group, prior bDMARDs use, treatment group-by-time interaction, with random slopes and random intercept.
^{b}Estimated linear rate of structural progression by Week 26 and standard errors are presented.
^{c}LS means and 95% CI based on a linear regression model fit to change from baseline in mTSS adjusting for treatment group, baseline mTSS, and geographic region.
^{d}Mean change from baseline and standard deviation are presented.

### Physical Function Response

Treatment with RINVOQ® 15 mg, alone or in combination with cDMARDs, resulted in a greater improvement in physical function at Week 12/14 compared to all comparators as measured by HAQ-DI.

### Other Health-Related Outcomes

In all trials except for Trial RA-V, patients receiving RINVOQ® 15 mg had greater improvement from baseline in physical component summary (PCS) score, mental component summary (MCS) scores, and in all 8 domains of the Short Form Health Survey (SF-36) compared to placebo in combination with cDMARDs or MTX monotherapy at Week 12/14.

Fatigue was assessed by the Functional Assessment of Chronic Illness Therapy-Fatigue score (FACIT-F) in Trials RA-I, RA-III, and RA-IV. Improvement in fatigue at Week 12 was observed in patients treated with RINVOQ® 15 mg compared to patients on placebo in combination with cDMARDs or MTX monotherapy.

### Psoriatic Arthritis

The efficacy and safety of RINVOQ® 15 mg once daily were assessed in two Phase 3 randomized, double-blind, multicenter, placebo-controlled studies in patients 18 years of age or older with moderately to severely active psoriatic arthritis. All patients had active psoriatic arthritis for at least 6 months based upon the Classification Criteria for Psoriatic Arthritis (CASPAR), at least 3 tender joints and at least 3 swollen joints, and active plaque psoriasis or history of plaque psoriasis. Although another dose has been studied, the recommended dose of RINVOQ® is 15 mg once daily for psoriatic arthritis.

Study PsA-I (NCT03104400) was a 24-week trial in 1705 patients with moderately to severely active psoriatic arthritis who had an inadequate response or intolerance to at least one non-biologic DMARD. Patients received RINVOQ® 15 mg or upadacitinib 30 mg once daily, adalimumab, or placebo, alone or in combination with background non-biologic DMARDs. At Week 24, all patients randomized to placebo were switched to RINVOQ® 15 mg or upadacitinib 30 mg once daily in a blinded manner. The primary endpoint was the proportion of patients who achieved an ACR20 response at Week 12.

Study PsA-II (NCT03104374) was a 24-week trial in 642 patients with moderately to severely active psoriatic arthritis who had an inadequate response or intolerance to at least one biologic DMARD. Patients received RINVOQ® 15 mg or upadacitinib 30 mg once daily or placebo, alone or in combination with background non-biologic DMARDs. At Week 24, all patients randomized to placebo were switched to RINVOQ® 15 mg or upadacitinib 30 mg once daily in a blinded manner. The primary endpoint was the proportion of patients who achieved an ACR20 response at Week 12.

### Clinical Response

In both studies, patients treated with RINVOQ® 15 mg achieved significantly higher ACR20 responses compared to placebo at Week 12 (Table 28, FIG. 2). A higher proportion of patients treated with RINVOQ® 15 mg achieved ACR50 and ACR70 responses at Week 12 compared to placebo. Treatment with RINVOQ® 15 mg resulted in improvements in the ACR components compared to placebo at the primary efficacy time point (Table 29).

TABLE 28
Clinical Response

| Study | | | |
|---|---|---|---|
| Study PsA-I | | Study PsA-II | |
| non-biologic DMARD-IR | | bDMARD-IR | |
| Treatment Group | | | |
| PBO | RINVOQ ® 15 mg | PBO | RINVOQ ® 15 mg |
| % | % Δ (95% CI) | % | % Δ (95% CI) |
| N | | | |
| 423 | 429 | 212 | 211 |

ACR20

| | | | | |
|---|---|---|---|---|
| Week 12 | 36 | 71 | 24 | 57 |
| | | 35 (28, 41) | | 33 (24, 42) |

ACR50

| | | | | |
|---|---|---|---|---|
| Week 12 | 13 | 38 | 5 | 32 |
| | | 24 (19, 30) | | 27 (20, 34) |

ACR70

| | | | | |
|---|---|---|---|---|
| Week 12 | 2 | 16 | 1 | 9 |
| | | 13 (10, 17) | | 8 (4, 12) |

Abbreviations:
ACR20 (or 50 or 70) = American College of Rheumatology ≥20% (or ≥50% or ≥70%) improvement,
bDMARD = biologic disease-modifying anti-rheumatic drug;
IR = inadequate responder;
PBO = placebo
Patients who discontinued randomized treatment or were missing data at week of evaluation were imputed as non-responders in the analyses.

TABLE 29
Components of ACR Response^{a}

| Study | | | |
|---|---|---|---|
| Study PsA-I | | Study PsA-II | |
| non-biologic DMARD-IR | | bDMARD-IR | |
| Treatment Group | | | |
| PBO | RINVOQ ® 15 mg | PBO | RINVOQ ® 15 mg |
| (N = 423) | (N = 429) | (N = 212) | (N = 211) |

Number of tender/painful joints (0-68)

| | | | | | | | | |
|---|---|---|---|---|---|---|---|---|
| Baseline | 20.0 | (14.3) | 20.4 | (14.7) | 25.3 | (17.6) | 24.9 | (17.3) |
| Week 12 | 12.5 | (13.3) | 8.8 | (12.5) | 19.3 | (18.5) | 12.6 | (15.6) |

Number of swollen joints (0-66)

| | | | | | | | | |
|---|---|---|---|---|---|---|---|---|
| Baseline | 11.0 | (8.2) | 11.6 | (9.3) | 12.0 | (8.9) | 11.3 | (8.2) |
| Week 12 | 5.6 | (7.2) | 3.5 | (6.0) | 7.3 | (9.4) | 4.4 | (5.7) |

Patient assessment of pain^{b}

| | | | | | | | | |
|---|---|---|---|---|---|---|---|---|
| Baseline | 6.1 | (2.1) | 6.2 | (2.1) | 6.6 | (2.1) | 6.4 | (2.1) |
| Week 12 | 5.1 | (2.3) | 3.8 | (2.4) | 5.9 | (2.3) | 4.4 | (2.5) |

Patient global assessment^{b}

| | | | | | | | | |
|---|---|---|---|---|---|---|---|---|
| Baseline | 6.3 | (2.0) | 6.6 | (2.0) | 6.8 | (2.0) | 6.8 | (1.9) |
| Week 12 | 5.2 | (2.2) | 3.8 | (2.3) | 6.1 | (2.3) | 4.5 | (2.5) |

Disability index (HAQ-DI)^{c}

| | | | | | | | | |
|---|---|---|---|---|---|---|---|---|
| Baseline | 1.1 | (0.6) | 1.2 | (0.7) | 1.2 | (0.7) | 1.1 | (0.6) |
| Week 12 | 1.0 | (0.7) | 0.7 | (0.6) | 1.1 | (0.6) | 0.8 | (0.7) |

Physician global assessment^{b}

| | | | | | | | | |
|---|---|---|---|---|---|---|---|---|
| Baseline | 6.5 | (1.6) | 6.7 | (1.6) | 6.5 | (1.8) | 6.5 | (1.8) |
| Week 12 | 4.3 | (2.2) | 3.1 | (2.0) | 5.0 | (2.2) | 3.4 | (2.1) |

hsCRP (mg/L)

| | | | | | | | | |
|---|---|---|---|---|---|---|---|---|
| Baseline | 11.5 | (15.8) | 11.0 | (14.9) | 10.4 | (18.5) | 11.2 | (18.6) |
| Week 12 | 10.1 | (15.2) | 4.2 | (9.9) | 9.4 | (13.4) | 4.3 | (7.9) |

Abbreviations:
ACR = American College of Rheumatology;
hsCRP = high sensitivity c-reactive protein;
HAQ-DI = Health Assessment Questionnaire-Disability Index;
IR = inadequate responder;
PBO = placebo
^{a}Data shown are mean (standard deviation).
^{b}Numeric rating scale (NRS): 0 = best, 10 = worst
^{c}Health Assessment Questionnaire-Disability Index: 0 = best, 3 = worst; 20 questions; 8 categories: dressing and grooming, arising, eating, walking, hygiene, reach, grip, and activities.

The percentage of patients achieving ACR20 response by visit is shown in FIG. 2.

Treatment with RINVOQ® 15 mg resulted in improvement in dactylitis and enthesitis in patients with pre-existing dactylitis or enthesitis.

Treatment with RINVOQ® 15 mg resulted in improvement in skin manifestations in patients with PsA. However, RINVOQ® has not been studied in and is not indicated for the treatment of plaque psoriasis.

### Physical Function Response

In both studies, patients treated with RINVOQ® 15 mg showed significant improvement in physical function from baseline compared to placebo as assessed by HAQ-DI at Week 12 (Table 26). The mean difference (95% CI) from placebo in HAQ-DI change from baseline at Week 12 was −0.28 (−0.35, −0.22) in Study PsA-I and −0.21 (−0.30, −0.12) in Study PsA-II.

The proportion of HAQ-DI responders (>0.35 improvement from baseline in HAQ-DI score) at Week 12 in Study PsA-I and Study PsA-II was 58% and 45%, respectively, in patients receiving RINVOQ® 15 mg and 33% and 27%, respectively, in patients receiving placebo.

### Radiographic Response

In Study PsA-I, inhibition of progression of structural damage was assessed radiographically and expressed as the change from baseline in modified Total Sharp Score (mTSS) and its components, the erosion score and the joint space narrowing score, at Week 24.

Treatment with RINVOQ® 15 mg inhibited progression of structural joint damage compared to placebo at Week 24 (Table 28). Analyses of erosion and joint space narrowing scores were consistent with overall results. The proportion of patients with no radiographic progression (mTSS change <0) at Week 24 was 93% in patients receiving RINVOQ® 15 mg and 89% in patients receiving placebo.

TABLE 30
Radiographic Changes in Study PsA-I

| | | |
|---|---|---|
| PBO | RINVOQ ® 15 mg | Estimated Difference |
| (N = 392) | (N = 407) | vs PBO at Week 24 |
| Mean (SD) | Mean (SD) | (95% CI)^{a} |

mTSS

| | | | |
|---|---|---|---|
| Baseline | 13.32 (31.2) | 13.14 (42.4) | |
| Week 24^{b} | 0.23 (0.07) | −0.02 (0.04) | −0.25 (−0.41, −0.09) |

Abbreviations:
CI = confidence intervals;
LS = least squares;
mTSS = modified Total Sharp Score;
PBO = placebo;
SD = standard deviation
^{a}LS means and 95% CI based on a random coefficient model fit to the mTSS value adjusting for time, treatment group, current DMARD use (yes/no), treatment group-by-time interaction, with random slopes and random intercept.
^{b}Estimated linear rate of structural progression by Week 24 and standard errors are presented.

### Other Health-Related Outcomes

Health-related quality of life was assessed by SF-36. In both studies, patients receiving RINVOQ® 15 mg experienced significantly greater improvement from baseline in the Physical Component Summary score compared to placebo at Week 12. Greater improvement was also observed in the Mental Component Summary score and all 8 domains of SF-36 compared to placebo.

Patients receiving RINVOQ® 15 mg showed greater improvement from baseline in fatigue, as measured by FACIT-F score, at Week 12 compared to placebo in both studies.

### Atopic Dermatitis

The efficacy of RINVOQ® 15 mg and 30 mg once daily, was assessed in three Phase 3 randomized, double-blind, multicenter trials (AD-1, AD-2, AD-3; NCT03569293, NCT03607422, and NCT03568318, respectively) in a total of 2584 patients (12 years of age and older). RINVOQ® was evaluated in 344 pediatric patients and 2240 adult patients with moderate to severe atopic dermatitis (AD) not adequately controlled by topical medication(s).

Disease severity at baseline was defined by a validated Investigator's Global Assessment (vIGA-AD) score ≥3 in the overall assessment of AD on a severity scale of 0 to 4, an Eczema Area and Severity Index (EASI) score ≥16, a minimum body surface area (BSA) involvement of ≥10%, and weekly average Worst Pruritus Numerical Rating Scale (NRS) score ≥4. Overall, 57% of the patients were male and 69% were white. The mean age at baseline was 34 years (ranged from 12 to 75 years) and 13% of the patients were 12 to less than 18 years. At baseline, 49% of patients had a vIGA-AD score of 3 (moderate AD), and 51% of patients had a vIGA-AD score of 4 (severe AD). The baseline mean EASI score was 29 and the baseline weekly average Worst Pruritus NRS score was 7. Approximately 52% of the patients had prior exposure to systemic AD treatment.

In all three trials, patients received RINVOQ® once daily oral doses of 15 mg, 30 mg, or matching placebo for 16 weeks. In Trial AD-3, patients also received RINVOQ® or placebo with concomitant topical corticosteroids (TCS) for 16 weeks.

All three trials assessed the co-primary endpoints of the proportion of patients with a vIGA-AD score of 0 (clear) or 1 (almost clear) with at least a 2-point improvement and the proportion of patients with EASI-75 (improvement of at least 75% in EASI score from baseline) at Week 16. Secondary endpoints included EASI-90 and EASI-100 at Week 16, and the proportion of patients with reduction in itch (>4-point improvement from baseline in the Worst Pruritus NRS) at Weeks 1, 4, and 16. In Trials AD-1 and AD-2, the proportion of patients with reduction in pain (>4-point improvement in the Atopic Dermatitis Symptom Scale [ADerm-SS] Skin Pain NRS) from baseline to Week 16 was a secondary endpoint.

Clinical Response

Monotherapy Trials (AD-1 and AD-2)

The results of RINVOQ® monotherapy trials (AD-1 and AD-2) are presented in Table 31. FIGS. 3A and 3B, respectively, present the proportion of patients with >4-point improvement in Worst Pruritus NRS at Weeks 1, 4, and 16 for Trials AD-1 and AD-2.

TABLE 31
Efficacy Results of Monotherapy Trials at Week 16 in Patients with Moderate to
Severe AD

| | Trial AD-1 | | | Trial AD-2 | | |
|---|---|---|---|---|---|---|
| | | RINVOQ ® | RINVOQ ® | | RINVOQ ® | RINVOQ ® |
| | PBO | 15 mg | 30 mg | PBO | 15 mg | 30 mg |
| Number of patients | 281 | 281 | 285 | 278 | 276 | 282 |
| randomized | | | | | | |
| vIGA-AD 0/1^{a,b} | 8% | 48% | 62% | 5% | 39% | 52% |
| Difference from | | 40% | 54% | | 34% | 47% |
| PBO (95% CI) | | (33%, 46%) | (47%, 60%) | | (28%, 40%) | (41%, 54%) |
| EASI-75^{a} | 16% | 70% | 80% | 13% | 60% | 73% |
| Difference from | | 53% | 63% | | 47% | 60% |
| PBO (95% CI) | | (46%, 60%) | (57%, 70%) | | (40%, 54%) | (53%, 66%) |
| EASI-90^{a} | 8% | 53% | 66% | 5% | 42% | 58% |
| Difference from | | 45% | 58% | | 37% | 53% |
| PBO (95% CI) | | (39%, 52%) | (51%, 64%) | | (31%, 43%) | (47%, 59%) |
| EASI-100^{a} | 2% | 17% | 27% | 1% | 14% | 19% |
| Difference from | | 15% | 25% | | 13% | 18% |
| PBO (95% CI) | | (10%, 20%) | (20%, 31%) | | (9%, 18%) | (13%, 23%) |
| Number of patients | | | | | | |
| with baseline Worst | 272 | 274 | 280 | 274 | 270 | 280 |
| Pruritus NRS score ≥ 4 | | | | | | |
| ≥4-point improvement | 12% | 52% | 60% | 9% | 42% | 60% |
| in Worst Pruritus NRS^{c} | | | | | | |
| Difference from | | 40% | 48% | | 33% | 50% |
| PBO (95% CI) | | (33%, 48%) | (41%, 55%) | | (26%, 39%) | (44%, 57%) |
| Number of patients | 233 | 237 | 249 | 247 | 237 | 238 |
| with baseline ADerm- | | | | | | |
| SS Skin Pain NRS | | | | | | |
| score ≥ 4 | | | | | | |
| ≥4-point improvement | 15% | 54% | 63% | 13% | 49% | 65% |
| in ADerm-SS Skin Pain | | | | | | |
| NRS^{d} | | | | | | |
| Difference from | | 39% | 49% | | 36% | 52% |
| PBO (95% CI) | | (31%, 47%) | (41%, 56%) | | (28%, 43%) | (44%, 59%) |

Abbreviations:
ADerm-SS = Atopic Dermatitis Symptom Scale;
PBO = placebo
^{a}Based on number of patients randomized
^{b}Responder was defined as a patient with vIGA-AD 0 or 1 (“clear” or “almost clear”) with a reduction of ≥2 points on a 0-4 ordinal scale
^{c}Based on number of patients whose baseline Worst Pruritus NRS is ≥4
^{d}Based on number of patients whose baseline ADerm-SS Skin Pain NRS is ≥4

Examination of age, gender, race, weight, and prior systemic treatment with immunosuppressants did not identify differences in response to RINVOQ® among these subgroups in Trials AD-1 and AD-2.

### Concomitant TCS Trial (AD-3)

The results of the RINVOQ® with concomitant TCS trial (AD-3) are presented in Table 32. FIG. 4 presents the proportion of patients with ≥4-point improvement in Worst Pruritus NRS at Weeks 1, 4, and 16 for Trial AD-3. Examination of age, gender, race, weight, and prior systemic treatment with immunosuppressants did not identify differences in response to RINVOQ® among these subgroups in Trial AD-3.

TABLE 32
Efficacy Results with Concomitant TCS at Week
16 in Patients with Moderate to Severe AD

| | Trial AD-3 | | |
|---|---|---|---|
| | | RINVOQ ® | RINVOQ ® |
| | PBO + TCS | 15 mg + TCS | 30 mg + TCS |
| Number of patients | 304 | 300 | 297 |
| randomized | | | |
| vIGA-AD 0/1^{a, b} | 11% | 40% | 59% |
| Difference from | | 29% | 48% |
| PBO (95% CI) | | (22%, 35%) | (41%, 54%) |
| EASI-75^{a,} | 26% | 65% | 77% |
| Difference from | | 38% | 51% |
| PBO (95% CI) | | (31%, 45%) | (44%, 57%) |
| EASI-90^{a} | 13% | 43% | 63% |
| Difference from | | 30% | 50% |
| PBO (95% CI) | | (23%, 36%) | (43%, 56%) |
| EASI-100^{a} | 1% | 12% | 23% |
| Difference from | | 11% | 21% |
| PBO (95% CI) | | (7%, 14%) | (16%, 26%) |
| Number of patients with | 294 | 288 | 291 |
| baseline Worst Pruritus | | | |
| NRS score ≥4 | | | |
| ≥4-point improvement in | 15% | 52% | 64% |
| Worst Pruritus NRS^{c} | | | |
| Difference from | | 37% | 49% |
| PBO (95% CI) | | (30%, 44%) | (42%, 56%) |

Abbreviations:
PBO = placebo
^{a}Based on number of patients randomized
^{b}Responder was defined as a patient with vIGA-AD 0 or 1 (“clear” or “almost clear”) with a reduction of ≥2 points on a 0-4 ordinal scale
^{c}Based on number of patients whose baseline Worst Pruritus NRS is ≥4

### Pediatric Patient Population

The efficacy results of the RINVOQ® monotherapy trials (AD-1 and AD-2) and the RINVOQ® with concomitant TCS trial (AD-3) at Week 16 for pediatric patients 12 years of age and older are presented in Table 33 and Table 34, respectively.

TABLE 33
Efficacy Results of Monotherapy Trials for Pediatric Patients 12 Years of Age and
Older with Moderate to Severe AD at Week 16

| | Trial AD-1 | | | Trial AD-2 | | |
|---|---|---|---|---|---|---|
| | | RINVOQ ® | RINVOQ ® | | RINVOQ ® | RINVOQ ® |
| | PBO | 15 mg | 30 mg | PRO | 15 mg | 30 mg |
| Number of pediatric | 40 | 42 | 42 | 36 | 33 | 35 |
| patients randomized | | | | | | |
| vIGA-AD 0/1^{a,b} | 8% | 38% | 69% | 3% | 42% | 62% |
| Difference from | | 31% | 62% | | 40% | 60% |
| PBO (95% CI) | | (14%, 47%) | (45%, 78%) | | (22%, 57%) | (42%, 77%) |
| EASI-75^{a} | 8% | 71% | 83% | 14% | 67% | 74% |
| Difference from | | 63% | 75% | | 53% | 61% |
| PBO (95% CI) | | (47%, 79%) | (61%, 89%) | | (33%, 72%) | (42%, 79%) |
| Number of pediatric | 39 | 40 | 42 | 36 | 30 | 34 |
| patients with baseline | | | | | | |
| Worst Pruritus NRS | | | | | | |
| score ≥ 4 | | | | | | |
| ≥4-point improvement | 15% | 45% | 55% | 3% | 33% | 50% |
| in Worst Pruritus NRS^{c} | | | | | | |
| Difference from | | 30% | 39% | | 31% | 47% |
| PBO (95% CI) | | (10%, 49%) | (21%, 58%) | | (13%, 48%) | (30%, 65%) |

Abbreviations:
PBO = placebo
^{a}Based on number of pediatric patients randomized
^{b}Responder was defined as a patient with vIGA-AD 0 or 1 (“clear” or “almost clear”) with a reduction of ≥2 points on a 0-4 ordinal scale
^{c}Based on number of pediatric patients whose baseline Worst Pruritus NRS is ≥4

TABLE 34
Efficacy Results with Concomitant TCS for Pediatric Patients 12
Years of Age and Older with Moderate to Severe AD at Week 16

| | Trial AD-3 | | |
|---|---|---|---|
| | | RINVOQ ® | RINVOQ ® |
| | PBO + TCS | 15 mg + TCS | 30 mg + TCS |
| Number of pediatric | 40 | 39 | 37 |
| patients randomized | | | |
| vIGA-AD 0/1^{a, b} | 8% | 31% | 65% |
| Difference from | | 23% | 57% |
| PBO (95% CI) | | (7%, 40%) | (40%, 75%) |
| EASI-75^{a} | 30% | 56% | 76% |
| Difference from | | 26% | 46% |
| PBO (95% CI) | | (5%, 47%) | (26%, 65%) |
| Number of pediatric | 38 | 36 | 33 |
| patients with | | | |
| baseline Worst Pruritus | | | |
| NRS score ≥4 | | | |
| ≥4-point improvement in | 13% | 42% | 55% |
| Worst Pruritus NRS^{c} | | | |
| Difference from | | 29% | 41% |
| PBO (95% CI) | | (9%, 48%) | (21%, 61%) |

Abbreviations:
PBO = placebo
^{a}Based on number of pediatric patients randomized
^{b}Responder was defined as a patient with vIGA-AD 0 or 1 (“clear” or “almost clear”) with a reduction of ≥2 points on a 0-4 ordinal scale
^{c}Based on number of pediatric patients whose baseline Worst Pruritus NRS is ≥4

### Ulcerative Colitis

### Induction Trials (Study UC-1 and Study UC-2)

In two identical induction trials (UC-1; NCT02809635 and UC-2; NCT03653026), patients were randomized 2:1 to receive either RINVOQ® 45 tug once daily or placebo for 8 weeks. A total of 988 patients were analyzed across the two trials. These trials included adult patients with moderately to severely active ulcerative colitis who had an inadequate response, loss of response, or intolerance to oral aminosalicylates, corticosteroids, immunosuppressants, and/or biologic therapy. Enrolled patients were permitted to use stable doses of oral aminosalicylates, methotrexate, ulcerative colitis-related antibiotics, and/or oral corticosteroids (up to 30 mg/day prednisone or equivalent). At baseline, 38% of patients were receiving corticosteroids, and 68% of patients were receiving aminosalicylates, Concomitant biologic therapies, azathioprine, 6-mercaptopurine, intravenous or rectal corticosteroids were prohibited. A total of 51% of patients had previously failed treatment with or were intolerant to at least one biologic therapy. RINVOQ® is indicated for patients who have an inadequate response or intolerance to one or more THE blockers.

Disease severity was assessed on the modified Mayo score (mMS), a 3-component Mayo score (0-9) which consists of the following subscores (0 to 3 for each subscore): stool frequency (SFS), rectal bleeding (RBS), and findings on centrally read endoscopy score (ES). An ES of 2 was defined by marked erythema, lack of vascular pattern, any friability, and/or erosions, and a score of 3 was defined by spontaneous bleeding and ulceration. Enrolled patients had a mMS between 5 to 9 with an ES of 2 or 3; at baseline the median mMS was 7, with 61% of patients having a baseline mMS of 5 to 7 and 39% having a mMS of 8 to 9.

At baseline, 39% and 37% of patients received corticosteroids, 1% and 1% of patients received methotrexate, and 68% and 69% of patients received aminosalicylates in UC-1 and UC-2, respectively. Patient disease severity was moderate (mMS ≤7) in 61% and 60% of patients and severe (mMS >7) in 39% and 40% of patients in UC-1 and UC-2, respectively.

The primary endpoint was clinical remission defined using the mMS at Week 8. Secondary endpoints included clinical response, endoscopic improvement, and histologic endoscopic mucosal improvement (see Table 35 and Table 36).

TABLE 35
Proportion of Patients Meeting Primary and Key
Secondary Efficacy Endpoints at Week 8 -Study UC-1
Study UC-1

| | | | |
|---|---|---|---|
| | | RINVOQ ® | Treatment Difference |
| | | 45 mg | vs Placebo |
| Endpoint | Placebo | Once Daily | (95% CI) |

Clinical Remission^{a}

| | | | |
|---|---|---|---|
| Total Population | N = 154 | N = 319 | 22%^{b} |
| | 5% | 26% | (16, 27) |
| Prior Biologic failure^{c} | N = 78 | N = 168 | |
| | <1% | 18% | |
| Without Prior Biologic | N = 76 | N = 151 | |
| Failure | 9% | 35% | |

Clinical Response^{d}

| | | | |
|---|---|---|---|
| Total Population | N = 154 | N = 319 | 46%^{b} |
| | 27% | 73% | (38, 54) |
| Prior biologic failure^{c} | N = 78 | N = 168 | |
| | 13% | 64% | |
| Without prior biologic | N = 76 | N = 151 | |
| failure | 42% | 82% | |

Endoscopic Improvement^{e}

| | | | |
|---|---|---|---|
| Total Population | N = 154 | N = 319 | 29%^{b} |
| | 7% | 36% | (23, 36) |
| Prior biologic failure^{c} | N = 78 | N = 168 | |
| | 2% | 27% | |
| Without prior biologic | N = 76 | N = 151 | |
| failure | 13% | 47% | |

Histologic Endoscopic Mucosal Improvement^{f}

| | | | |
|---|---|---|---|
| Total Population | N = 154 | N = 319 | 24%^{b} |
| | 7% | 30% | (17, 30) |
| Prior biologic failure^{c} | N = 78 | N = 168 | |
| | 1% | 23% | |
| Without prior biologic | N = 76 | N = 151 | |
| failure | 12% | 38% | |

^{a}Per mMS: SFS ≤ 1 and not greater than baseline, RBS = 0, ES of ≤1 without friability
^{b}p < 0.001, adjusted treatment difference (95% CI) based on Cochran-Mantel-Haenszel method adjusted for randomization stratification factors
^{c}Prior biologic failure includes inadequate response, loss of response, or intolerance to one or more biologic treatments for ulcerative colitis.
^{d}Per mMS: decrease ≥2 points and ≥30% from baseline and a decrease in RBS ≥ 1 from baseline or an absolute RBS ≤ 1
^{e}ES ≤1 without friability
^{f}ES ≤1 without friability and Geboes score ≤ 3.1 (indicating neutrophil infiltration in <5% of crypts, no crypt destruction and no erosions, ulcerations or granulation tissue)

TABLE 36
Proportion of Patients Meeting Primary and Key
Secondary Efficacy Endpoints at Week 8 -Study UC-2
Study UC-2

| | | | |
|---|---|---|---|
| | | RINVOQ ® | Treatment Difference |
| | | 45 mg | vs Placebo |
| Endpoint | Placebo | Once Daily | (95% CI) |

Clinical Remission^{a}

| | | | |
|---|---|---|---|
| Total Population | N = 174 | N = 341 | 29% ^{b} |
| | 4% | 33% | (23, 35) |
| Prior Biologic Failure^{c} | N = 89 | N = 173 | |
| | 2% | 30% | |
| Without Prior Biologic | N = 85 | N = 168 | |
| Failure | 6% | 38% | |

Clinical Response^{d}

| | | | |
|---|---|---|---|
| Total Population | N = 174 | N = 341 | 49% ^{b} |
| | 25% | 74% | (42, 57) |
| Prior biologic failure^{c} | N = 89 | N = 173 | |
| | 19% | 69% | |
| Without prior biologic | N = 85 | N = 168 | |
| failure | 32% | 80% | |

Endoscopic Improvement^{e}

| | | | |
|---|---|---|---|
| Total Population | N = 174 | N = 341 | 35% ^{b} |
| | 8% | 44% | (29, 42) |
| Prior biologic failure^{c} | N = 89 | N = 173 | |
| | 5% | 37% | |
| Without prior biologic | N = 85 | N = 168 | |
| failure | 12% | 51% | |

Histologic Endoscopic Mucosal Improvement^{f}

| | | | |
|---|---|---|---|
| Total Population | N = 174 | N = 341 | 30% ^{b} |
| | 6% | 37% | (24, 36) |
| Prior biologic failure^{c} | N = 89 | N = 173 | |
| | 5% | 31% | |
| Without prior biologic | N = 85 | N = 168 | |
| failure | 7% | 43% | |

^{a}Per mMS: SFS ≤ 1 and not greater than baseline, RBS = 0, ES of ≤1 without friability
^{b} p < 0.001, adjusted treatment difference (95% CI) based on Cochran-Mantel-Haenszel method adjusted for randomization stratification factors
^{c}Prior biologic failure includes inadequate response, loss of response, or intolerance to one or more biologic treatments for ulcerative colitis.
^{d}Per mMS: decrease ≥2 points and ≥30% from baseline and a decrease in RBS ≥ 1 from baseline or an absolute RBS ≤ 1
^{e}ES ≤1 without friability
^{f}ES ≤1 without friability and Geboes score ≤ 3.1 (indicating neutrophil infiltration in <5% of crypts, no crypt destruction and no erosions, ulcerations or granulation tissue)

Studies UC-1 and UC-2 were not designed to evaluate the relationship of histologic endoscopic mucosal improvement at week 8 to disease progression and long-term outcomes.

### Rectal Bleeding and Stool Frequency Subscores

Onset of clinical response was assessed using the SFS and RBS (partial modified Mayo Score [pmMS]). Initial response was defined as a decrease of ≥1 point and ≥30% from Baseline in pmMS and a decrease in RBS ≥1 or an absolute RBS≤1. Onset of response occurred as early as Week 2 in a greater proportion of patients treated with RINVOQ® 45 mg once daily compared to placebo.

### Endoscopic and Histologic Assessment

Normalization of the endoscopic appearance of the mucosa (endoscopic remission) was defined as ES of 0. At Week 8, a greater proportion of patients treated with RINVOQ® 45 mg once daily compared to placebo achieved endoscopic remission (UC-1: 14% vs 1%, UC-2: 18% vs 2%). Endoscopic remission with Geboes histologic score <2.0 (indicating no neutrophils in crypts or lamina propria and no increase in eosinophil, no crypt destruction, and no erosions, ulcerations, or granulation tissue) was achieved by a greater proportion of patients treated with RINVOQ® 45 mg once daily compared to placebo at Week 8 (UC-1: 11% vs 1%, UC-2: 13% vs 2%).

### Abdominal Pain and Bowel Urgency

A greater proportion of patients treated with RINVOQ® 45 mg once daily compared to placebo had no abdominal pain (UC-1: 47% vs 23%, UC-2: 54% vs 24%) and no bowel urgency (UC-1: 48% vs 21%, UC-2: 54% vs 26%) at Week 8.

### Maintenance Study UC-3

In UC-3 (NCT02819635), a total of 451 patients who received RINVOQ® 45 mg once daily in either UC-1, UC-2 or UC-4 and achieved clinical response were re-randomized to receive RINVOQ® 15 mg, 30 mg or placebo once daily for up to 52 weeks.

The primary endpoint was clinical remission defined using mMS at Week 52. Secondary endpoints included corticosteroid-free clinical remission, endoscopic improvement, and histologic endoscopic mucosal improvement (see Table 37).

TABLE 37
Proportion of Patients Meeting Efficacy Endpoints at Week 52 in Maintenance Study UC-3

| | | | | | |
|---|---|---|---|---|---|
| | | RINVOQ ® | Treatment Difference | RINVOQ ® | Treatment Difference |
| | | 15 mg | 15 mg vs Placebo | 30 mg | 30 mg vs Placebo |
| Endpoint | Placebo | Once Daily | (95% CI) | Once Daily | (95% CI) |

Clinical remission^{a}

| | | | | | |
|---|---|---|---|---|---|
| Total Population | N = 149 | N = 148 | 31% ^{b} | N = 154 | 39% ^{b} |
| | 12% | 42% | (22, 40) | 52% | (30, 48) |
| Prior biologic failure^{c} | N = 81 | N = 71 | | N = 73 | |
| | 7% | 41% | | 49% | |
| Without prior biologic | N = 68 | N = 77 | | N = 81 | |
| failure⁺ | 18% | 44% | | 54% | |

Corticosteroid-free clinical remission^{d}

| | | | | | |
|---|---|---|---|---|---|
| Total Population | N = 54 | N = 47 | 35% ^{b} | N = 58 | 45% ^{b} |
| | 22% | 57% | (18, 53) | 68% | (29, 62) |
| Prior biologic failure^{c} | N = 22 | N = 17 | | N = 20 | |
| | 14% | 71% | | 73% | |
| Without prior biologic | N = 32 | N = 30 | | N = 38 | |
| failure | 28% | 49% | | 65% | |

Endoscopic Improvement^{e}

| | | | | | |
|---|---|---|---|---|---|
| Total Population | N = 149 | N = 148 | 34% ^{b} | N = 154 | 46% ^{b} |
| | 14% | 49% | (25, 44) | 62% | (37, 56) |
| Prior biologic failure^{c} | N = 81 | N = 71 | | N = 73 | |
| | 8% | 43% | | 56% | |
| Without prior biologic | N = 68 | N = 77 | | N = 81 | |
| failure⁺ | 22% | 54% | | 67% | |

Histologic Endoscopic Mucosal Improvement^{f}

| | | | | | |
|---|---|---|---|---|---|
| Total Population | N = 149 | N = 148 | 24% ^{b} | N = 154 | 37% ^{b} |
| | 12% | 35% | (15, 33) | 50% | (28, 47) |
| Prior biologic failure^{c} | N = 81 | N = 71 | | N = 73 | |
| | 5% | 33% | | 48% | |
| Without prior biologic | N = 68 | N = 77 | | N = 81 | |
| failure | 20% | 37% | | 52% | |

^{a}Per mMS: SFS ≤ 1 and not greater than baseline, RBS = 0, ES ≤1 without friability
^{b} p < 0.001, adjusted treatment difference (95% CI) based on Cochran-Mantel-Haenszel method adjusted for randomization stratification factors
^{c}Prior biologic failure includes inadequate response, loss of response, or intolerance to one or more biologic treatments for ulcerative colitis.
^{d}Clinical remission per mMS at Week 52 and corticosteroid free for ≥90 days immediately preceding Week 52 among patients who achieved clinical remission at the end of the induction treatment
^{e}ES ≤1 without friability
^{f}ES ≤1 without friability and Geboes score ≤ 3.1 (indicating neutrophil infiltration in <5% of crypts, no crypt destruction and no erosions, ulcerations or granulation tissue)

The relationship between histologic endoscopic mucosal improvement at Week 52 and disease progression and longer-term outcomes after Week 52 was not evaluated in Study UC-3.

### Endoscopic and Histologic Assessment

Normalization of the endoscopic appearance of the mucosa (endoscopic remission) was defined as ES of 0. In UC-3, a m greater proportion of patients treated with RINVOQ® 15 mg and 30 mg once daily compared to placebo achieved endoscopic remission at Week 52 (24% and 26% vs 6%). Endoscopic remission with Geboes histologic score <2.0 was achieved by a greater proportion of patients treated with RINVOQ® 15 mg and 30 mg once daily compared to placebo at Week 52 (18% and 19% vs 5%).

### Abdominal Pain and Bowel Urgency

At Week 52, a greater proportion of patients treated with RINVOQ® 15 mg and 30 mg once daily compared to placebo had no abdominal pain (46%, 55% and 21%, respectively) and no bowel urgency (56%, 64% and 17%, respectively).

### How Supplied/Storage and Handling

### How Supplied

RINVOQ® extended-release tablets are supplied as:
15 mg: purple, biconvex oblong, with dimensions of 14×8 mm, and debossed with ‘a15’ on one side. 30 tablets in a bottle; NDC: 0074-2306-30
30 mg: red, biconvex oblong, with dimensions of 14×8 mm, and debossed with ‘a30’ on one side. 30 tablets in a bottle; NDC: 0074-2310-30
45 mg: yellow to mottled yellow, biconvex oblong, with dimensions of 14×8 mm, and debossed with ‘a45’ on one side. 28 tablets in a bottle; NDC: 0074-1043-28

### Storage and Handling

Store at 2° C. to 25° C. (36° F. to 77° F.).

Store in the original bottle in order to protect from moisture.

### Patient Counseling Information

Advise the patient to read the FDA-approved patient labeling (Medication Guide).

### Serious Infections

Inform patients that they may be more likely to develop infections when taking RINVOQ®. Instruct patients to contact their healthcare provider immediately during treatment if they develop any signs or symptoms of an infection. Advise patients that the risk of herpes zoster is increased in patients taking RINVOQ® and in some cases can be serious.

### Malignancies

Inform patients that RINVOQ® may increase their risk of certain cancers and that periodic skin examinations should be performed while using RINVOQ®. Advise patients that exposure to sunlight and UV light should be limited by wearing protective clothing and using a broad-spectrum sunscreen.

### Major Adverse Cardiovascular Events

Inform patients that RINVOQ® may increase their risk of major adverse cardiovascular events (MACE) including myocardial infarction, stroke, and cardiovascular death. Instruct all patients, especially current or past smokers or patients with other cardiovascular risk factors, to be alert for the development of signs and symptoms of cardiovascular events.

### Thrombosis

Inform patients that events of deep venous thrombosis and pulmonary embolism have been reported in clinical trials with RINVOQ®. Instruct patients to seek immediate medical attention if they develop any signs or symptoms of a DVT or PE.

### Hypersensitivity Reactions

Advise patients to discontinue RINVOQ® and seek immediate medical attention if they develop any signs and symptoms of allergic reactions.

### Gastrointestinal Perforations

Inform patients that gastrointestinal perforations have been reported in clinical trials with RINVOQ® and that risk factors include the use of NSAIDS or history of diverticulitis. Instruct patients to seek medical care immediately if they experience new onset of abdominal pain, fever, chills, nausea, or vomiting.

### Retinal Detachment

Inform patients that retinal detachment has been reported in clinical trials with RINVOQ®. Advise patients to immediately inform their healthcare provider if they develop any sudden changes in vision while receiving RINVOQ®.

### Laboratory Abnormalities

Inform patients that RINVOQ® may affect certain lab tests, and that blood tests are required before and during RINVOQ® treatment.

### Vaccinations

Advise patients to avoid use of live vaccines with RINVOQ®. Instruct patients to inform their healthcare practitioner that they are taking RINVOQ® prior to a potential vaccination.

### Embryo-Fetal Toxicity

Advise pregnant women and females of reproductive potential that exposure to RINVOQ® during pregnancy may result in fetal harm. Advise females to inform their healthcare provider of a known or suspected pregnancy. Advise females of reproductive potential that effective contraception should be used during treatment and for 4 weeks following the final dose of upadacitinib.

### Lactation

Advise women not to breastfeed during treatment with RINVOQ® and for 6 days after the last dose.

### Administration

Advise patients not to chew, crush, or split RINVOQ® tablets.

### Medication Guide

RINVOQ® (RIN-VOKE) extended-release tablets, for oral use. What is the most important information I should know about RINVOQ®? RINVOQ® can cause serious side effects, including:

### 1. Serious Infections.

RINVOQ® is a medicine that affects your immune system. RINVOQ® can lower the ability of your immune system to fight infections. Some people have had serious infections while taking RINVOQ®, including tuberculosis (TB), and infections caused by bacteria, fungi, or viruses that can spread throughout the body. Some people have died from these infections.
Your healthcare provider should test you for TB before starting treatment with RINVOQ®.
Your healthcare provider should watch you closely for signs and symptoms of TB during treatment with RINVOQ®.
You should not start taking RINVOQ® if you have any kind of infection unless your healthcare provider tells you it is okay. You may be at a higher risk of developing shingles (herpes zoster).
Before starting RINVOQ®, tell your healthcare provider if you:
   are being treated for an infection.
   have had an infection that does not go away or that keeps coming back.
   have diabetes, chronic lung disease, HIV, or a weak immune system.
   have TB or have been in close contact with someone with TB.
   have had shingles (herpes zoster).
   have or have had hepatitis B or C.
   live or have lived, or have traveled to certain parts of the country (such as the Ohio and Mississippi River valleys and the Southwest) where there is an increased chance for getting certain kinds of fungal infections. These infections may happen or become more severe if you use RINVOQ®. Ask your healthcare provider if you do not know if you have lived in an area where these infections are common.
   think you have an infection or have symptoms of an infection such as:
      fever, sweating, or chills
      muscle aches
      cough
      shortness of breath
      feeling tired
      weight loss
      warm, red, or painful skin or sores on your body
      blood in your phlegm
      diarrhea or stomach pain
      burning when you urinate or urinating more often than usual

After starting RINVOQ®, call your healthcare provider right away if you have any symptoms of an infection. RINVOQ® can make you more likely to get infections or make worse any infections that you have. If you get a serious infection, your healthcare provider may stop your treatment with RINVOQ® until your infection is controlled.

2. Increased Risk of Death in People 50 Years of Age and Older Who have at Least 1 Heart Disease (Cardiovascular) Risk Factor and are Taking a Medicine in the Class of Medicines Called Janus Kinase (JAK) Inhibitors. RINVOQ® is a JAK Inhibitor Medicine.

### 3. Cancer and Immune System Problems.

RINVOQ® may increase your risk of certain cancers by changing the way your immune system works. Lymphoma and other cancers, including skin cancers can happen in people taking RINVOQ®. People taking a medicine in the class of medicines called Janus kinase (JAK) inhibitors have a higher risk of certain cancers including lymphoma and lung cancer, especially if you are a current or past smoker. Tell your healthcare provider if you have ever had any type of cancer. Follow your healthcare provider's advice about having your skin checked for skin cancer during treatment with RINVOQ®. Limit the amount of time you spend in sunlight. Avoid using tanning beds or sunlamps. Wear protective clothing when you are in the sun and use a sunscreen with a high protection factor (SPF 30 and above). This is especially important if your skin is very fair or if you have a family history of skin cancer.

4. Increased Risk of Major Cardiovascular Events Such as Heart Attack, Stroke or Death in People 50 Years of Age and Older Who have at Least 1 Heart Disease (Cardiovascular) Risk Factor and Taking a Medicine in the Class of Medicines Called JAK Inhibitors, Especially if You are a Current or Past Smoker.

Get emergency help right away if you have any symptoms of a heart attack or stroke while taking RINVOQ®, including:
discomfort in the center of your chest that lasts for more than a few minutes, or that goes away and comes back
severe tightness, pain, pressure, or heaviness in your chest, throat, neck, or jaw
pain or discomfort in your arms, back, neck, jaw, or stomach
shortness of breath with or without chest discomfort
breaking out in a cold sweat
nausea or vomiting
feeling lightheaded
weakness in one part or on one side of your body
slurred speech

### 5. Blood Clots (Thrombosis).

Blood clots in the veins of your legs (deep vein thrombosis, DVT) or lungs (pulmonary embolism, PE) and arteries (arterial thrombosis) can happen in some people taking RINVOQ®. This may be life-threatening and cause death. Blood clots in the veins of the legs (DVT) and lungs (PE) have happened more often in people who are 50 years of age and older and with at least 1 heart disease (cardiovascular) risk factor taking a medicine in the class of medicines called Janus kinase (JAK) inhibitors. Tell your healthcare provider if you have had blood clots in the veins of your legs or lungs in the past. Get medical help right away if you have signs and symptoms of blood clots during treatment with RINVOQ®, including:
swelling
sudden unexplained chest or upper back pain
pain or tenderness in one or both legs
shortness of breath or difficulty breathing

### 6. Allergic Reactions.

Symptoms such as rash (hives), trouble breathing, feeling faint or dizzy, or swelling of your lips, tongue, or throat, that may mean you are having an allergic reaction have been seen in people taking RINVOQ®. Some of these reactions were serious. If any of these symptoms occur during treatment with RINVOQ®, stop taking RINVOQ® and get emergency medical help right away.

### 7. Tears (Perforation) in the Stomach or Intestines.

Tell your healthcare provider if you have had diverticulitis (inflammation in parts of the large intestine) or ulcers in your stomach or intestines. Some people taking RINVOQ® can get tears in their stomach or intestines. This happens most often in people who take nonsteroidal anti-inflammatory drugs (NSAIDs), corticosteroids, or methotrexate. Get medical help right away if you get stomach-area pain, fever, chills, nausea, or vomiting.

### 8. Changes in Certain Laboratory Test Results.

Your healthcare provider should do blood tests before you start taking RINVOQ® and while you take RINVOQ® to check for the following:
low neutrophil and lymphocyte counts. Neutrophils and lymphocytes are types of white blood cells that help the body fight off infections.
low red blood cell counts. Red blood cells carry oxygen. Low red blood cells means you may have anemia, which may make you feel weak and tired.
increased cholesterol levels. Your healthcare provider should do blood tests to check your cholesterol levels approximately 12 weeks after you start taking RINVOQ®, and as needed.
elevated liver enzymes. Liver enzymes help to tell if your liver is functioning normally. Elevated liver enzymes may indicate that your healthcare provider needs to do additional tests on your liver.

You should not take RINVOQ® if your neutrophil count, lymphocyte count, or red blood cell count is too low or your liver tests are too high. Your healthcare provider may stop your RINVOQ® treatment for a period of time if needed because of changes in these blood test results. See “What are the possible side effects of RINVOQ®?” for more information about side effects. What is RINVOQ®?

RINVOQ® is a prescription medicine that is a Janus kinase (JAK) inhibitor. RINVOQ® is used:
to treat adults with moderate to severe rheumatoid arthritis when 1 or more medicines called tumor necrosis factor (TNF) blockers have been used, and did not work well or could not be tolerated.
to treat adults with active psoriatic arthritis when 1 or more medicines called tumor necrosis factor (TNF) blockers have been used, and did not work well or could not be tolerated.
to treat adults and children 12 years of age and older with moderate to severe eczema (atopic dermatitis) that did not respond to previous treatment and their eczema is not well controlled with other pills or injections, including biologic medicines, or the use of other pills or injections is not recommended.

RINVOQ® is safe and effective in children 12 years of age and older weighing at least 88 pounds (40 kg) with atopic dermatitis. It is not known if RINVOQ® is safe and effective in children under 18 years of age with juvenile idiopathic arthritis or with psoriatic arthritis. It is not known if RINVOQ® is safe and effective in children under 12 years of age with atopic dermatitis. Do not take RINVOQ® if you are allergic to upadacitinib or any of the ingredients in RINVOQ®. See the end of this Medication Guide for a complete list of ingredients in RINVOQ®.

Before taking RINVOQ®, tell your healthcare provider about all of your medical conditions, including if you:
See “What is the most important information I should know about RINVOQ®?”
have an infection.
are a current or past smoker.
have had a heart attack, other heart problems, or stroke.
have liver problems.
have kidney problems.
have unexplained stomach (abdominal) pain, have a history of diverticulitis or ulcers in your stomach or intestines, or are taking NSAIDs.
have low red or white blood cell counts.
have recently received or are scheduled to receive an immunization (vaccine). People who take RINVOQ® should not receive live vaccines.
are pregnant or plan to become pregnant. Based on animal studies, RINVOQ® may harm your unborn baby.
   Females who are able to become pregnant:
Your healthcare provider will check whether or not you are pregnant before you start treatment with RINVOQ®.
You should use effective birth control (contraception) to avoid becoming pregnant during treatment with RINVOQ® and for 4 weeks after your last dose of RINVOQ®.
Tell your healthcare provider if you think you are pregnant or become pregnant during treatment with RINVOQ®.
If you take RINVOQ® during pregnancy, contact AbbVie Inc. at 1-800-633-9110, or FDA at 1-800-FDA-1088 or www.fda.gov/medwatch to provide information about the health of you and your baby.
are breastfeeding or plan to breastfeed. RINVOQ® may pass into your breast milk. You and your healthcare provider should decide if you will take RINVOQ® or breastfeed. Do not breastfeed during treatment with RINVOQ® and for 6 days after your last dose of RINVOQ®.

Tell your healthcare provider about all the medicines you take, including prescription and over-the-counter medicines, vitamins, and herbal supplements. RINVOQ® and other medicines may affect each other causing side effects.

Especially tell your healthcare provider if you take:
medicines for fungal infections (such as ketoconazole, itraconazole, posaconazole or voriconazole) or clarithromycin (for bacterial infections) as these medicines may increase the amount of RINVOQ® in your blood.
rifampicin (for bacterial infections) or phenytoin (for neurological disorders) as these medicines may decrease the effect of RINVOQ®.
medicines that affect your immune system (such as azathioprine and cyclosporine) as these medicines may increase your risk of infection.

Ask your healthcare provider or pharmacist, if you are not sure if you are taking any of these medicines. Know the medicines you take. Keep a list of them to show your healthcare provider and pharmacist when you get a new medicine.

How should I take RINVOQ®?
Take RINVOQ® exactly as your healthcare provider tells you to use it.
Take RINVOQ® 1 time a day with or without food.
Swallow RINVOQ® tablets whole. Do not split, crush, or chew the tablets.
If you take too much RINVOQ®, call your healthcare provider or poison control center at 1-800-222-1222, or go to the nearest hospital emergency room right away.
   What are the possible side effects of RINVOQ®?
   RINVOQ® may cause serious side effects, including:

See “What is the most important information I should know about RINVOQ®?”

The most common side effects of RINVOQ® in people treated for rheumatoid arthritis and psoriatic arthritis include:
upper respiratory tract infections (common cold, sinus infections)
shingles (herpes zoster)
herpes simplex virus infections, including cold sores
bronchitis
nausea
cough
fever
acne

The most common side effects of RINVOQ® in people treated for atopic dermatitis include:
upper respiratory tract infections (common cold, sinus infections)
acne
herpes simplex virus infections, including cold sores
headache
increased blood levels of creatine phosphokinase
cough
allergic reactions
inflammation of hair follicles
nausea
stomach-area (abdominal) pain
fever
increased weight
shingles (herpes zoster)
flu
tiredness
low white blood cell count (neutropenia)
muscle pain
flu-like illness

Separation or tear to the lining of the back part of the eye (retinal detachment) has happened in people with atopic dermatitis treated with RINVOQ®. Call your healthcare provider right away if you have any sudden changes in your vision during treatment with RINVOQ®.

These are not all the possible side effects of RINVOQ®. Call your doctor for medical advice about side effects.

How should I store RINVOQ®?

Store RINVOQ® at 36° F. to 77° F. (2° C. to 25° C.).

Store RINVOQ® in the original bottle to protect it from moisture.

Keep RINVOQ® and all medicines out of the reach of children.

General Information about the Safe and Effective Use of RINVOQ®.

Medicines are sometimes prescribed for purposes other than those listed in a Medication Guide. Do not use RINVOQ® for a condition for which it was not prescribed. Do not give RINVOQ® to other people, even if they have the same symptoms that you have. It may harm them.

You can ask your pharmacist or healthcare provider for information about RINVOQ® that is written for health professionals.

### What are the Ingredients in RINVOQ® 15 mg Tablets?

Active ingredient: upadacitinib.
Inactive ingredients: colloidal silicon dioxide, ferrosoferric oxide, hypromellose, iron oxide red, magnesium stearate, mannitol, microcrystalline cellulose, polyvinyl alcohol, polyethylene glycol, talc, tartaric acid and titanium dioxide.

### What are the Ingredients in RINVOQ® 30 mg Tablets?

Active ingredient: upadacitinib
Inactive ingredients: colloidal silicon dioxide, hypromellose, iron oxide red, magnesium stearate, mannitol, microcrystalline cellulose, polyvinyl alcohol, polyethylene glycol, talc, tartaric acid and titanium dioxide.

### Example 3. Impact of Strong CYP3A4 Inhibitors on the Pharmacokinetics of Upadacitinib

As described herein above, strong CYP3A4 inhibitors increase exposure and Cmax of upadacitinib when administered concurrently. Specifically, Study M13-401 assessed the impact of strong CYP3A4 inhibitors on the pharmacokinetics of upadacitinib and showed that co-administration of a strong CYP3A4 inhibitor resulted in 70% and 75% higher Cmax and AUC_{inf}, respectively, of upadacitinib. The data from Study M13-401 are provided in Table 38 and Table 39, which show the expected increase in Cₘₐₓ and AUCₛₛ, respectively, in patients with UC across all dose levels with co-administration of CYP3A4 inhibitors.

TABLE 38
Model-Estimated Upadacitinib Cₘₐₓ (ng/mL) in Patients
with UC and Predicted Cₘₐₓ in Patients with UC and
Co-administration of Strong CYP3A4 Inhibitors

| | | | | Predicted Median | |
|---|---|---|---|---|---|
| | | | | Cₘₐₓ (ng/mL) in | |
| | | | | UC Patients with | |
| | | Model-Estimated | | Strong CYP3A4 inhibitors | |
| | | Cₘₐₓ (ng/mL) from | | based on results | |
| | | Phase 2b and 3 UC Studies | | from M13-401 | |
| Treatment | | Median | | Median | |
| Group | | (5^{th}, 95^{th} Percentile) | | (5^{th}, 95^{th} Percentile) | |
| 7.5 | mg | 20.2 | (15.3, 32.8) | 34.3 | (26.0, 55.8) |
| 15 | mg | 41.3 | (26.3, 57.2) | 70.2 | (44.7, 97.2) |
| 30 | mg | 78.5 | (57.3, 121) | 133 | (97.4, 206) |
| 45 | mg | 121 | (86.2, 182) | 206 | (147, 309) |

*Predicted median, 5^{th}, and 95^{th} percentiles in UC patients using strong CYP3A inhibitors were calculated by multiplying the observed effect in Study M13-401 (1.7) by the model-estimated median, 5^{th}, and 95^{th} percentiles for Cₘₐₓ in subjects with UC in Phase 2b and 3 trials.

TABLE 39
Model-Estimated Upadacitinib AUCₛₛ (ng/mL) in Patients
with UC and Predicted AUCₛₛ in Patients with UC
and Co-administration of Strong CYP3A Inhibitors

| | | | | Predicted | |
|---|---|---|---|---|---|
| | | | | AUCₛₛ (ng · h/mL) in | |
| | | | | UC Patients with | |
| | | Model-Estimated | | Strong CYP3A4 inhibitors | |
| | | AUCₛₛ (ng · h/mL) from | | based on results | |
| | | Phase 2b and 3 UC Studies | | from M13-401 | |
| Treatment | | Median | | Median | |
| Group | | (5^{th}, 95^{th} Percentile) | | (5^{th}, 95^{th} Percentile) | |
| 7.5 | mg | 161 | (114, 355) | 282 | (200, 621) |
| 15 | mg | 342 | (216, 629) | 599 | (378, 1101) |
| 30 | mg | 653 | (432, 1142) | 1143 | (756, 1999) |
| 45 | mg | 994 | (631, 1682) | 1740 | (1104, 2944) |

*Predicted median, 5^{th}, and 95^{th} percentiles in UC patients using strong CYP3A inhibitors were calculated by multiplying the observed effect in Study M13-401 (1.75) by the model-estimated median, 5^{th}, and 95^{th} percentiles for AUCₛₛ in subjects with UC in Phase 2b and 3 trials.

Table 38 and Table 39 show that a dose of 45 mg QD, when co-administered with strong CYP3A4 inhibitors in UC patients, would result in upadacitinib Cₘₐₓ and AUC values which exceed the range of upadacitinib plasma exposures evaluated during the induction Phase 2b and 3 trials in a large proportion of patients. If the dose is reduced to 30 mg QD, upadacitinib plasma exposures in the majority of patients are predicted to be within plasma exposures observed in the Phase 2b and 3 induction trials for 45 mg QD.

Similarly for maintenance, a dose of 30 mg QD, when co-administered with strong CYP3A4 inhibitors, is predicted to result in upadacitinib Cₘₐₓ and AUC values which exceed the range of plasma exposures evaluated during the maintenance trial for 30 mg QD for a large proportion of patients. If the dose is reduced to 15 mg QD, upadacitinib plasma exposures are predicted to be within the plasma exposures evaluated in the maintenance Phase 3 trial for 30 mg QD. Therefore, based on these results, the recommended dose of upadacitinb in patients with UC, when a strong CYP3A4 inhibitor is concurrently administered, is 30 mg once daily during induction treatment and 15 mg once daily during maintenance treatment.

### Example 4. Impact of Renal Impairment on the Pharmacokinetics of Upadacitinib

As described herein above, the presence of severe renal impairment in a patient increases exposure and Cmax of upadacitinib in said patient. Specifically, Study M13-551 assessed the impact of renal impairment on the pharmacokinetics of upadacitinib. The results from the study showed that severe renal impairment resulted in 14% and 44% higher Cₘₐₓ and AUC_{inf}, respectively, compared to subjects with normal renal function. Upadacitinib predicted plasma exposures in UC patients who have severe renal impairment are shown in Table 40 for Cₘₐₓ and in Table 41 for AUC, based on the observed effect of severe renal impairment in study M13-551 and the model-estimated plasma exposures in patients with UC in Phase 2b and 3 trials.

TABLE 40
Model-Estimated Upadacitinib Cₘₐₓ (ng/mL) in Patients with UC and
Predicted Cₘₐₓ in Patients with UC and Severe Renal Impairment

| | | | | Predicted | |
|---|---|---|---|---|---|
| | | | | Cₘₐₓ (ng/mL) in | |
| | | | | UC Patients with | |
| | | Model-Estimated | | Severe Renal Impairment | |
| | | Cₘₐₓ (ng/mL) from | | based on results | |
| | | Phase 2b and 3 UC Studies | | from M13-551 | |
| Treatment | | Median | | Median | |
| Group | | (5^{th}, 95^{th} Percentile) | | (5^{th}, 95^{th} Percentile)* | |
| 7.5 | mg | 20.2 | (15.3, 32.8) | 23.0 | (17.4, 37.4) |
| 15 | mg | 41.3 | (26.3, 57.2) | 47.1 | (30.0, 65.2) |
| 30 | mg | 78.5 | (57.3, 121) | 89.5 | (65.3, 138) |
| 45 | mg | 121 | (86.2, 182) | 138 | (98.3, 208) |

*Predicted median, 5^{th}, and 95^{th} percentiles in UC patients with severe renal impairment were calculated by multiplying the observed effect in Study M13-551 (1.14) by the model-estimated median, 5^{th}, and 95^{th} percentiles for Cₘₐₓ in subjects with UC in Phase 2b and 3 trials.

TABLE 41
Model-Estimated Upadacitinib AUCₛₛ (ng/mL)
in Patients with UC and Predicted AUCₛₛ in
Patients with UC and Severe Renal Impairment

| | | | | Predicted | |
|---|---|---|---|---|---|
| | | | | AUCₛₛ (ng · h/mL) in | |
| | | | | UC Patients with | |
| | | Model-Estimated | | Severe Renal Impairment | |
| | | AUCₛₛ (ng · h/mL) from | | based on results | |
| | | Phase 2b and 3 UC Studies | | from M13-551 | |
| Treatment | | Median | | Median | |
| Group | | (5^{th}, 95^{th} Percentile) | | (5^{th}, 95^{th} Percentile) | |
| 7.5 | mg | 161 | (114, 355) | 232 | (164, 511) |
| 15 | mg | 342 | (216, 629) | 492 | (311, 906) |
| 30 | mg | 653 | (432, 1142) | 940 | (622, 1645) |
| 45 | mg | 994 | (631, 1682) | 1431 | (909, 2422) |

*Predicted median, 5^{th}, and 95^{th} percentiles in UC patients with severe renal impairment were calculated by multiplying the observed effect in Study M13-551 (1.44) by the model-estimated median, 5^{th}, and 95^{th} percentiles for AUCₛₛ in subjects with UC in Phase 2b and 3 trials.

The dosing recommendations for upadacitinib in special populations is determined based on the magnitude of increase in upadacitinib plasma exposures relative to the range of model-estimated upadacitinib plasma exposures evaluated during induction and maintenance in the Phase 2b and 3 studies. Table 40 and Table 41 show that a dose of 45 mg QD administered to UC patients with severe renal impairment will result in Cmax values which are within the range observed in the Phase 2b and 3 studies. However, many of these patients will have upadacitinib AUC that exceeds the 95th percentile for upadacitinib AUC in Phase 2b and 3 induction trials (>1,682 ng·h/mL). Therefore, it is recommended that the induction dose is reduced in UC patients with severe renal impairment to 30 mg QD. Upadacitinib Cmax and AUC for a dose of 30 mg QD in severe renal impairment patients are predicted to be within the plasma exposures of subjects with UC in the Phase 2b and 3 trials (Table 41 and Table 42).

For maintenance, a upadacitinib dose of 30 mg QD administered to patients with severe renal impairment is predicted to result in Cmax values which are within the range observed during maintenance in the Phase 3 maintenance study. However, if UC patients with severe renal impairment receive 30 mg QD dose upadacitinib for maintenance, upadacitinib AUC is predicted to exceed the 95th percentile of the exposures observed in the maintenance study for 30 mg QD in a significant proportion of patients. Therefore, it is recommended that the maintenance dose is reduced in UC patients with severe renal impairment to 15 mg QD. Upadacitinib Cmax and AUC for a dose of 15 mg QD in severe renal impairment patients are predicted to be within the plasma exposures of exposures of patients with UC in the Phase 3 maintenance trial.

In summary, based on the results presented here, the recommended dose for patients with UC and severe renal impairment is 30 mg once daily during induction treatment and 15 mg once daily during maintenance treatment.

1. A method of treating a patient concurrently receiving a strong CYP3A4 inhibitor with a therapeutically effective amount of upadacitinib, the method comprising:
if the recommended daily upadacitinib dose for the patient is 45 mg in the absence of a strong CYP3A4 inhibitor, administering 30 mg to the patient during the time the patient is receiving the strong CYP3A4 inhibitor;
if the recommended daily upadacitinib dose for the patient is 30 mg in the absence of a strong CYP3A4 inhibitor, administering 15 mg to the patient during the time the patient is receiving the strong CYP3A4 inhibitor; and
if the recommended daily upadacitinib dose for the patient is 15 mg in the absence of a strong CYP3A4 inhibitor, administering 15 mg to the patient during the time the patient is receiving the strong CYP3A4 inhibitor.

2. The method of claim 1, wherein the patient for whom the recommended daily upadacitinib dose is 45 mg is an adult having moderately to severely active ulcerative colitis, and the daily upadacitinib dose of 30 mg is an induction dose administered for 8 weeks.

3. The method of claim 1, wherein the patient for whom the recommended daily upadacitinib dose is 45 mg is an adult having moderately to severely active Crohn's disease, and the daily upadacitinib dose of 30 mg is an induction dose administered for 8 to 16 weeks.

4. The method of claim 1, wherein the patient for whom the recommended daily upadacitinib dose is 30 mg is an adult refractory, severe or extensive ulcerative colitis, and the daily upadacitinib dose of 15 mg is a maintenance dose.

5. The method of claim 1, wherein the patient for whom the recommended daily upadacitinib dose is 30 mg is an adult having refractory, severe or extensive Crohn's disease, and the daily upadacitinib dose of 15 mg is a maintenance dose.

6. The method of claim 1, wherein the patient for whom the recommended daily upadacitinib dose is 30 mg is a pediatric patient 12 years of age and older weighing at least 40 kg having atopic dermatitis that did not achieve adequate response with a daily upadacitinib dose of 15 mg, or an adult less than 65 years of age having atopic dermatitis that did not achieve adequate response with a daily upadacitinib induction dose of 15 mg.

7. The method of claim 1, wherein the patient for whom the recommended daily upadacitinib dose is 15 mg is an adult having moderately to severely active rheumatoid arthritis, an adult having active psoriatic arthritis, an adult or pediatric patient 12 years of age and older having refractory, moderate to severe atopic dermatitis, or an adult having active axial spondyloarthritis.

8. The method of claim 1, wherein the CYP3A4 inhibitor is ketoconazole.

9. The method of claim 1, wherein 400 mg of the ketoconazole is administered once daily for 6 days.

10. A method of treating a patient concurrently receiving a strong CYP3A4 inhibitor with a therapeutically effective amount of upadacitinib, the method comprising:
if the recommended daily upadacitinib dose for the patient is 45 mg in the absence of a strong CYP3A4 inhibitor, administering 30 mg to the patient during the time the patient is receiving the strong CYP3A4 inhibitor; or
if the recommended daily upadacitinib dose for the patient is 30 mg in the absence of a strong CYP3A4 inhibitor, administering 15 mg to the patient during the time the patient is receiving the strong CYP3A4 inhibitor.

11. The method of claim 10, wherein the patient is an adult having moderately to severely active ulcerative colitis and the recommended daily upadacitinib dose for the patient is 45 mg, the method comprising administering an once daily upadacitinib induction dose of 30 mg for 8 weeks.

12. The method of claim 10, wherein the patient is an adult having moderately to severely active Crohn's disease and the recommended daily upadacitinib dose for the patient is 45 mg, the method comprising administering a once daily upadacitinib induction dose of 30 mg for 8 to 16 weeks.

13. The method of claim 10, wherein the patient is an adult having refractory, severe or extensive ulcerative colitis and the recommended daily upadacitinib dose for the patient is 30 mg, the method comprising administering a once daily upadacitinib maintenance dose of 15 mg.

14. The method of claim 10, wherein the patient is an adult having refractory, severe or extensive Crohn's disease and the recommended daily upadacitinib dose of 30 mg, the method comprising administering a once daily upadacitinib maintenance dose of 15 mg.

15. The method of claim 10, wherein the patient is a pediatric patient 12 years of age and older weighing at least 40 kg having atopic dermatitis that did not achieve adequate response with a daily upadacitinib dose of 15 mg, or an adult less than 65 years of age having atopic dermatitis that did not achieve adequate response with a daily upadacitinib induction dose of 15 mg, the method comprising administering a once daily upadacitinib dose of 15 mg.

16. The method of claim 10, wherein the CYP3A4 inhibitor is ketoconazole.

17. The method of claim 10, wherein 400 mg of the ketoconazole is administered once daily for 6 days.

18. A method of treating patients having severe renal impairment with a therapeutically effective amount of upadacitinib, the method comprising:
if the recommended daily upadacitinib dose for the patient is 45 mg in the absence of severe renal impairment, administering 30 mg to the patient during the time the patient has severe renal impairment;
if the recommended daily upadacitinib dose for the patient is 30 mg in the absence of severe renal impairment, administering 15 mg to the patient during the time the patient has severe renal impairment; and
if the recommended daily upadacitinib dose for the patient is 15 mg in the absence of severe renal impairment, administering 15 mg to the patient during the time the patient has severe renal impairment.

19. The method of claim 18, wherein the patient for whom the recommended daily upadacitinib dose is 45 mg is an adult having moderately to severely active ulcerative colitis, and the daily upadacitinib dose of 30 mg is an induction dose administered for 8 weeks.

20. The method of claim 18, wherein the patient for whom the recommended daily upadacitinib dose is 45 mg is an adult having moderately to severely active Crohn's disease, and the daily upadacitinib dose of 30 mg is an induction dose administered for 8 to 16 weeks.

21. The method of claim 18, wherein the patient for whom the recommended daily upadacitinib dose is 30 mg is an adult refractory, severe or extensive ulcerative colitis, and the daily upadacitinib dose of 15 mg is a maintenance dose.

22. The method of claim 18, wherein the patient for whom the recommended daily upadacitinib dose is 30 mg is an adult having refractory, severe or extensive Crohn's disease, and the daily upadacitinib dose of 15 mg is a maintenance dose.

23. The method of claim 18, wherein the patient for whom the recommended daily upadacitinib dose is 30 mg is a pediatric patient 12 years of age and older weighing at least 40 kg having atopic dermatitis that did not achieve adequate response with a daily upadacitinib dose of 15 mg, or an adult less than 65 years of age having atopic dermatitis that did not achieve adequate response with a daily upadacitinib induction dose of 15 mg.

24. The method of claim 18, wherein the patient for whom the recommended daily upadacitinib dose is 15 mg is an adult having moderately to severely active rheumatoid arthritis, an adult having active psoriatic arthritis, an adult or pediatric patient 12 years of age and older having refractory, moderate to severe atopic dermatitis, or an adult having active axial spondyloarthritis.

25. A method of treating a patient having severe renal impairment with a therapeutically effective amount of upadacitinib, the method comprising:
if the recommended daily upadacitinib dose for the patient is 45 mg in the absence of a severe renal impairment, administering 30 mg to the patient during the time the patient has severe renal impairment; or
if the recommended daily upadacitinib dose for the patient is 30 mg in the absence of severe renal impairment, administering 15 mg to the patient during the time the patient has severe renal impairment.

26. The method of claim 25, wherein the patient is an adult having moderately to severely active ulcerative colitis and the recommended daily upadacitinib dose for the patient is 45 mg, the method comprising administering a once daily upadacitinib induction dose of 30 mg for 8 weeks.

27. The method of claim 25, wherein the patient is an adult having moderately to severely active Crohn's disease and the recommended daily upadacitinib dose for the patient is 45 mg, the method comprising administering a once daily upadacitinib induction dose of 30 mg for 8 to 16 weeks.

28. The method of claim 25, wherein the patient is an adult having refractory, severe or extensive ulcerative colitis and the recommended daily upadacitinib dose for the patient is 30 mg, the method comprising administering a once daily upadacitinib maintenance dose of 15 mg.

29. The method of claim 25, wherein the patient is an adult having refractory, severe or extensive Crohn's disease and the recommended daily upadacitinib dose of 30 mg, the method comprising administering a once daily upadacitinib maintenance dose of 15 mg.

30. The method of claim 25, wherein the patient is a pediatric patient 12 years of age and older weighing at least 40 kg having atopic dermatitis that did not achieve adequate response with a daily upadacitinib dose of 15 mg, or an adult less than 65 years of age having atopic dermatitis that did not achieve adequate response with a daily upadacitinib induction dose of 15 mg, the method comprising administering a once daily upadacitinib dose of 15 mg.
